(19) United States
(12) Patent Application Publication
(10) Pub. No.: US 2021/0340504 A1
(43) Pub. Date: Nov. 4, 2021

(54) Cells and Methods for the Production of Ursodeoxycholic Acid and Precursors Thereof

(71) Applicant: INTREXON CORPORATION, Blacksburg, VA (US)

(72) Inventors: Maria ENQUIST-NEWMAN, Radford, VA (US); Erin TOM, Radford, VA (US); Cleo HO, Radford, VA (US); Christopher SAVILE, Radford, VA (US); Abhinav KUMAN, Radford, VA (US); Lauren ESSER, Radford, VA (US); Andrea CHAN, Radford, VA (US); Michael CLAY, Radford, VA (US); Adrianna PIGULA, Radford, VA (US); Hsiang-yun CHEN, Radford, VA (US)

(21) Appl. No.: 17/283,112

(22) PCT Filed: Oct. 8, 2019
(86) PCT No.: PCT/US2019/055180

§ 371 Date: Apr. 6, 2021

Related U.S. Application Data
(60) Provisional application No. 62/743,122, filed on Oct. 9, 2018.

(51) Int. Cl. C12N 9/04; C12N 15/52; C12P 33/06

(52) U.S. Cl. CPC C12N 9/0006; C12Y 101/01159; C12P 33/06; C12N 15/52

(57) ABSTRACT

Genetically-modified cell capable of producing UD CA, cholic acid, and/or another UDCA precursor comprising at least one heterologous polynucleotide encoding an enzyme involved in a metabolic pathway that converts sugar to UDCA, cholic acid, and/or another UDCA precursor. Method of making UDCA, cholic acid, and/or another UDCA precursor using such a cell. Use of UDCA or UDCA precursor produced using such a method for the manufacture of a medicament for the treatment of a disease or symptom of a disease. Medicament comprising UDCA or UDCA precursor made using such a method. Method of treating a disease or symptom of a disease comprising administering UDCA or a UDCA precursor made using such a method. Isolated nucleic acid encoding at least one enzyme involved in a metabolic pathway that converts sugar to UDCA, cholic acid, and/or another UDCA precursor. Vector comprising a nucleic acid encoding at least one enzyme involved in a metabolic pathway that converts sugar to UDCA, cholic acid and/or another UDCA precursor. Method of making a genetically-modified cell capable of synthesizing UDCA, cholic acid, and/or another UDCA precursor. Composition comprising UDCA or a UDCA precursor, a free acid or CoA thereof, or a pharmaceutically-acceptable derivative or prodrug thereof.

### BACKGROUND OF THE INVENTION

The subject matter of the present invention relates to microorganisms, such as yeast and bacteria, genetically-modified so as to produce ursodeoxycholic acid (“UDCA”) or a UDCA precursor. UDCA, also known as ursodiol, is a secondary bile acid produced in bears. Secondary bile acids are formed when primary bile acids produced by the liver are secreted into the intestines and metabolized by intestinal bacteria.

UDCA helps regulate cholesterol by reducing the rate at which the intestine absorbs cholesterol molecules while breaking up micelles containing cholesterol. Thus, UDCA is used to non-surgically treat gallstones made of cholesterol. It is also used to relieve itching in pregnancy for some women who suffer obstetric cholestasis. Additionally, UDCA can be used to treat primary biliary cirrhosis (PDC).

UDCA has never been directly produced by any known microbial system. See e.g., Tonin, F., and Arends, I. W. C. E., “Latest development in the synthesis of ursodeoxycholic acid (UDCA): a critical review,” Beilstein J. Otg. Chem. 14:470-483 (2018); see also e.g., Russell, D.W., “The enzymes, regulation, and genetics of bile acid synthesis,” Annu Rev Biochem 72:134-74 (2003). It is currently synthetized from animal-derived starting material at substantial costs. There is thus a need to produce UDCA cheaper and more efficiently.

Microbes in the human gut are known to produce UDCA by metabolizing chenodeoxycholic acid (CDCA), one of two primary bile acids produced by the human liver, where it is synthesized from cholesterol. However, microbes do not produce CDCA. It is thus desirable to engineer a cell or microorganism to produce CDCA, which may be useful inofitself or as an intermediate to the production of UDCA.

UDCA may also be produced chemically from cholic acid, the other primary bile acid produced by the human liver and synthesized from cholesterol. Cholic acid itself may be used to treat patients with bile acid or preoxisomal disorders. In addition, cholic acid may serve as a starting substrate for the synthesis of various other chemicals besides UDCA, including the secondary bile acid deoxycholic acid, which has various medicinal uses, such as a fat emulsifier and as a treatment for double chin.

Cholic acid, however, is currently obtained from the slaughter of animals, and the process of isolating the compound is often difficult and/or costly. Like CDCA, cholic acid is not known to be produced by microorganisms. It is thus desirable to engineer a cell or microorganism to produce cholic acid, which may be useful inofitself or as an intermediate to the production of other useful chemicals.

### SUMMARY OF THE INVENTION

The present invention relates in part to a genetically-modified cell capable of producing UDCA or a UDCA precursor. The cell may comprise at least one heterologous enzyme involved in a metabolic pathway that converts sugar to UDCA or a UDCA precursor and/or at least one heterologous polynucleotide encoding such an enzyme.

The invention also relates to a method of making UDCA or a UDCA precursor. The method comprises contacting a substrate with the aforementioned genetically-modified cell and growing the cell to make UDCA or UDCA precursor.

The invention further relates to the use of UDCA or UDCA precursor for the manufacture of a medicament for the treatment of a disease or a symptom of a disease and to such a medicament.

The invention additionally relates to a method of treating a disease or symptom of a disease comprising administering UDCA or a UDCA precursor to a subject in need thereof.

Yet another aspect of the invention is a nucleic acid encoding at least one enzyme involved in a metabolic pathway that converts sugar to UDCA or a UDCA precursor or a vector encoding such a nucleic acid.

A further aspect of the invention is a method of making a genetically-modified cell capable of synthesizing UDCA or a UDCA precursor, the method comprising: contacting a cell with at least one heterologous polynucleotide encoding an enzyme involved in a metabolic pathway that converts sugar to UDCA or a UDCA precursor; and growing the cell so that said enzyme is expressed in said microorganism.

A yet further aspect of the invention is a composition comprising UDCA or a UDCA precursor, a free acid or CoA thereof, or a pharmaceutically-acceptable derivative or prodrug thereof.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a 13-step enzymatic pathway from cholesterol to UDCA. The genes encoding this 13-step enzymatic pathway, which include CYP7A1, HSD3B7, AKR1D1, AKR1C4, CYP27A1, SLC27A5, Racemase, ACOX2, HSD17B4, Peroxisomal Thiolase 2, 7α-HSD, 7β-HSD, and choloyl-CoA hydrolase, were introduced into yeast.

FIG. 2 shows 2-step enzymatic pathway from cholesta-5,7,24-trienol, a native yeast sterol, to cholesterol. The genes encoding this 2-step enzymatic pathway include DHCR7 and DHCR24.

FIG. 3 shows the steps for preparing samples for mass spectroscopy analysis. The genetically-modified microorganisms described throughout were subject to this protocol in order to determine levels of UDCA and/or UDCA precursors made.

FIG. 4 shows two alternative methods for preparing samples for mass spectroscopy analysis. The genetically-modified microorganisms described throughout were subject to this protocol in order to determine levels of UDCA and/or UDCA precursors made.

FIG. 5 shows the amount of relative cholesterol made from yeast strains expressing various DHCR24 variants. The DHCR24 variants from Homo sapiens and Danio rerio (zebrafish) exhibited the best activities.

FIG. 6 shows the activities of CYP7A1 variants in making 7-alpha-hydroxycholesterol from cholesterol. CYP7A1 from Mus musculus exhibited the best activity.

FIG. 7 shows the activities of HSD3B7 variants in making 7α-hydroxy-4-cholesten-3-one from 7-alpha-hydroxycholesterol. HSD3B7 from Homo sapiens exhibited the best activity.

FIG. 8 shows the activities of AKR1D1 variants in making 7α-hydroxy-5β-cholestan-3-one from 7α-hydroxy-4-cholesten-3-one. AKR1D1 from Homo sapiens and Mus musculus exhibited the best activity

FIG. 9 shows the activities of AKR1C4 variants in making 5β-cholestane-3α,7α-diol from 7α-hydroxy-5β-cholestan-3-one. AKR1C4 from Macaca fuscata exhibited the best activity.

FIG. 10 shows the activities of CYP8B1 variants in making 7α,12α-dihydroxy-4-cholesten-3-one from 7α-hydroxy-4-cholesten-3-one. CYP8B1 from Mus musculus and Ogctolagus cuniculus exhibited the best activity.

FIG. 11 shows the activities of CYP27A1 variants in making (25R)-3α,7α-dihydroxy-5β-cholestanoic acid from 5β-cholestane-3α,7α-diol. In order to more easily detect CYP27A1 activity, SLC27A5 from Homo sapiens was introduced into the strains and the SLC27A5 product was measured by mass spec. Most of the variants were able to produce the SLC27A5 product.

FIGS. 12A and 12B show CoA ligase activities on (25R)-3α,7α,12α-trihydroxy-5β-cholestan-26-oic acid when expressing different variants of SLC27A5. FIG. 12A shows HPLC data indicating that there is a peak detected that is specific to ligase expressing strains. FIG. 12B shows mass spec data confirming the presence of active ligase in the expressing strains. It is also noted that CoA ligase also exhibits activity using 3α,5β,7α,12α,24E-trihydroxy-cholest-24-en-26-oic acid as the substrate.

FIGS. 13A and 13B show the activities of AMACR and ACOX2 variants in making different products. FIG. 13A shows AMACR from both Homo sapiens and Rattus norvegicus exhibit excellent racemization activity, converting (25R)-3α,7α-dihydroxy-5β-cholestanoyl-CoA into (25S)-3α,7α-dihydroxy-5β-cholestanoyl-CoA. FIG. 13B shows that ACOX2 from Homo sapiens in combination with Homo sapien AMACR has the best activity with respect to converting (25S)-3α,7α-dihydroxy-5β-cholestanoyl-CoA into (24E)-3α,7α-dihydroxy-5β-cholest-24-enoyl-CoA.

FIG. 14 shows the activities of ACOX2 variants in making (24E)-3α,7α-dihydroxy-5β-cholest-24-enoyl-CoA from (25S)-3α,7α-dihydroxy-5β-cholestanoyl-CoA. ACOX2 from Homo sapiens and Ogctolagus cuniculus exhibited the best activity.

FIG. 15 shows the activities of HSD17B4 variants in making 3α,7α(-dihydroxy-24-oxo-5β-cholestanoyl-CoA from (24E)-3α,7α-dihydroxy-5β-cholest-24-enoyl-CoA. HSD17B4 from Rattus norvegicus, Bos taurus, and Xenopus laevis exhibited the best activities.

FIG. 16 shows the activities of SCP2 variants in making 3α,7α(-dihydroxy-5β-cholan-24-oyl-CoA from 3α,7α(-dihydroxy-24-oxo-5β-cholestanoyl-CoA. SCP2 activity was detected by LCMS in all samples, including negative control. However, enhanced activity was observed in the strain overexpressing the native yeast gene POT1.

FIG. 17 shows the activities of 7α-HSD variants in making 3α-hydroxy-7-oxo-5β-cholan-24-oyl-CoA from 3α,7α-dihydroxy-5β-cholan-24-oyl-CoA. 7α-HSD from Escherichia coli and Bacteroides fragilis exhibited the best activity.

FIG. 18 shows the activities of 7β-HSD variants in making 3α,7β-dihydroxy-5β-cholan-24-oyl-CoA from 3α-hydroxy-7-oxo-5β-cholan-24-oyl-CoA. 7β-HSD from Clostridium sardiniense exhibited the best activity.

FIG. 19 shows the activities of several combinations of thiolase/SCP2, 7α-HSD, and 7β-HSD. The strains were then tested by GC/MS for the ability to produce UDCA/UDC-CoA. The following combinations exhibited the best activities: POT1 Thiolase, Escco (E. coli) 7α-HSD; and Closa (C. sardiniense) 7β-HSD and POT1 Thiolase, Bacfr (B. fragilis) 7α-HSD, and C. sardiniense 7β-HSD.

FIG. 20 shows the various enzymes involved in a pathway described herein for producing UDCA from sugar, the product of each of the enzymes, and the corresponding CoA and free acid forms of these products, where applicable. The CoA and the free acid forms are made by the microorganisms and the methods described throughout.

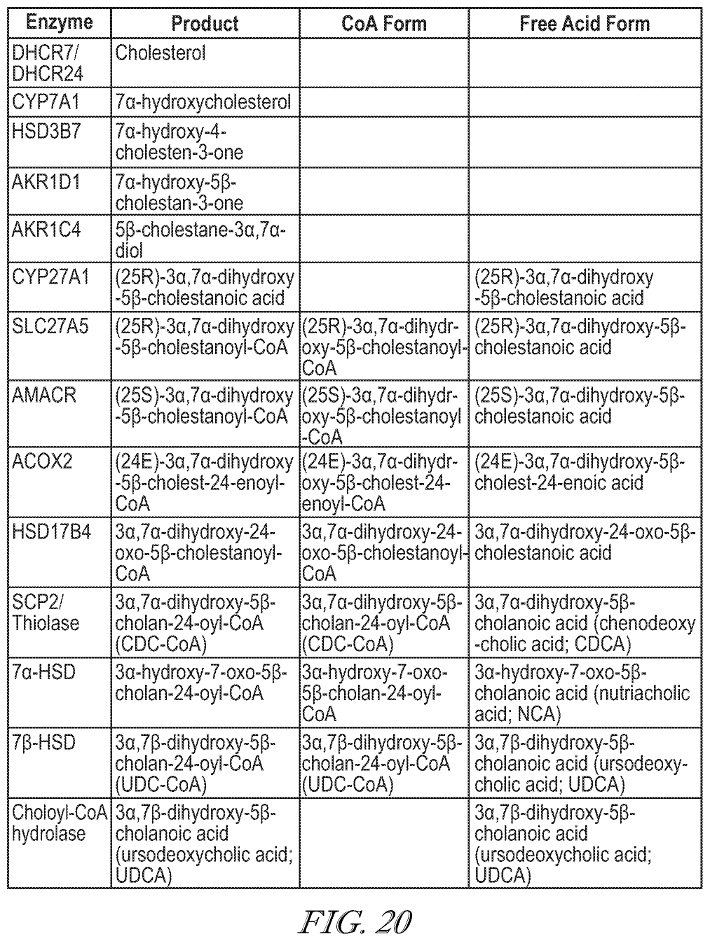

FIG. 21 shows a 12-step enzymatic pathway from cholesterol to cholic acid. The genes encoding this 12-step enzymatic pathway, which include CYP7A1, HSD3B7, CYP8B1, AKR1D1, AKR1C4, CYP27A1, SLC27A5, Racemase, ACOX2, HSD17B4, Peroxisomal Thiolase 2, and choloyl-CoA hydrolase, were introduced into yeast.

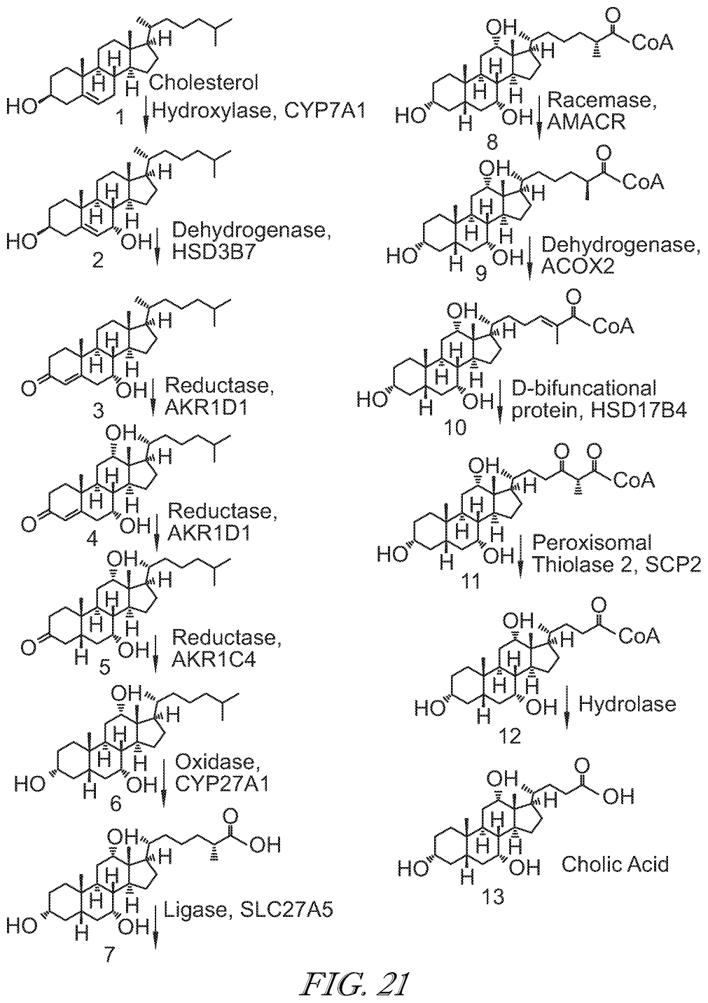

FIG. 22 shows the various enzymes involved in a pathway described herein for producing cholic acid from sugar, the product of each of the enzymes, and the corresponding CoA and free acid forms of these products, where applicable. The CoA and the free acid forms are made by the microorganisms and the methods described throughout.

FIG. 23 shows the activities of CYP8B1 variants in making 7α,12α-dihydroxy-4-cholesten-3-one from 7α-hydroxy-4-cholesten-3-one. CYP8B1 from Mus musculus and Ogctolagus cuniculus exhibited the best activity.

FIG. 24 depicts a flow chart showing the steps for performing liquid chromatography and mass spectrometry on a product.

FIG. 25 shows the amount of relative cholic acid detected from a yeast strain expressing CYP8B1 from Mus musculus and a yeast strain not expressing CYP8B1. The results show that CYP8B1 from Mus musculus was active and produced choloyl-CoA (cholic acid detected). No cholic acid was detected in the strain lacking the CYP8B1 enzyme.

### DETAILED DESCRIPTION OF THE INVENTION

Definitions

The term “about” in relation to a reference numerical value and its grammatical equivalents as used herein includes the numerical value itself and a range of values plus or minus 10% from that numerical value. For example, the amount “about 10” includes 10 and any amounts from 9 to 11.

The terms “genetic modification ” or “genetically-modified” and their grammatical equivalents as used herein refers to one or more alterations of a nucleic acid or to a cell that contains modifications to its genome.

The terms “operably connected”, “operably coupled”, and their grammatical equivalents are used herein interchangeably and refer to two or more units that work together to result in a certain outcome. For example, in reference to gene expression, a polynucleotide encoding a promoter can be operably connected to a polynucleotide encoding gene which, under the right conditions, can lead to the expression of the gene. With regard to a metabolic pathway, the term operably connected can refer to two or more enzymes that work in the pathway to convert a substrate into a product. The enzymes can be consecutive within the pathway. In some cases, the enzymes are not directly consecutive within the pathway.

The terms “and/or” and “any combination thereof” and their grammatical equivalents are used herein interchangeably and convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.”

The term “sugar” and its grammatical equivalents as used herein include, but are not limited to, (i) simple carbohydrates, such as monosaccharides (e.g., glucose fructose, galactose, ribose); disaccharides (e.g., maltose, sucrose, lactose); oligosaccharides (e.g., raffinose, stachyose); or (ii) complex carbohydrates, such as starch (e.g., long chains of glucose, amylose, amylopectin); glycogen; fiber (e.g., cellulose, hemicellulose, pectin, gum, mucilage).

The term “alcohol” and its grammatical equivalents as used herein include, but are not limited to, any organic compound in which the hydroxyl functional group (—OH) is bound to a saturated carbon atom. For example, the term alcohol encompasses: monohydric alcohols (e.g., methanol, ethanol, isopropyl alcohol, butanol, pentanol, cetyl alcohol); polyhydric alcohols (e.g., ethylene glycol, propylene glycol, glycerol, erythritol, threitol, xylitol, mannitol, sorbitol, volemitol);

unsaturated aliphatic alcohols (e.g., allyl alcohol, geraniol, propargyl alcohol); and alicyclic alcohols (e.g., inositol, menthol).

The term “fatty acid” and its grammatical equivalents as used herein include, but are not limited to, a carboxylic acid with a long aliphatic chain that is either saturated or unsaturated. Examples of unsaturated fatty acids include, but are not limited to, myristoleic acid, sapienic acid, linoelaidic acid, α-linolenic acid, stearidonic acid, eicosapentaenoic acid, docosahexaenoic acid, linoleic acid, γ-linolenic acid, dihomo-γ-linolenic acid, arachidonic acid, docosatetraenoic acid, palmitoleic acid, vaccenic acid, paullinic acid, oleic acid, elaidic acid, gondoic acid, erucic acid, nervonic acid, and mead acid. Examples of saturated fatty acids include, but are not limited to, propionic acid, butyric acid, valeric acid, hexanoic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, hexatriacontylic acid, heptatriacontanoic acid, and octatriacontanoic acid.

The term “substantially pure” and its grammatical equivalents as used herein mean that a particular substance does not contain a majority of another substance. For example, “substantially pure UDCA” can mean that the substance comprises at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, 99.99%, 99.999%, or 99.9999% UDCA.

The term “heterologous” and its grammatical equivalents as used herein means that a substance is derived from a different species than that of the host microorganism. For example, a “heterologous gene” means that the gene catedis from a different species than that of the host microorganism.

The term “substantially identical” and its grammatical equivalents as used herein in reference to sequences means that the sequences are at least 50% identical. In some instances, the term substantially identical refers to a sequence that is at least 55%, at least 60%, at least 65%, at least 70%, at least 75%, at least 80%, at least 85%, at least 90%, at least 95%, at least 96%, at least 97%, at least 98%, or at least 99% identical to the reference sequence. The percentage of identity between two sequences is determined by aligning the two sequences, using for example the alignment method of Needleman and Wunsch (J. Mol. Biol., 1970, 48: 443), as revised by Smith and Waterman (Adv. Appl. Math., 1981, 2: 482), so that the highest order match is obtained between the two sequences and the number of identical amino acids/nucleotides is determined between the two sequences. Methods to calculate the percentage identity between two amino acid sequences are generally art recognized and include, for example, those described by Carillo and Lipton (SIAM J. Applied Math., 1988, 48:1073) and those described in Computational Molecular Biology, Lesk, e.d. Oxford University Press, New York, 1988, Biocomputing: Informatics and Genomics Projects. Generally, computer programs will be employed for such calculations. Computer programs that may be used in this regard include, but are not limited to, GCG (Devereux et al., Nucleic Acids Res., 1984, 12: 387) BLASTP, BLASTN and FASTA (Altschul et al., J. Molec. Biol., 1990:215:403). A particularly preferred method for determining the percentage identity between two polypeptides involves the Clustal W algorithm (Thompson, J D, Higgines,

D G and Gibson T J, 1994, Nucleic Acid Res 22(22): 4673-4680 together with the BLOSUM 62 scoring matrix (Henikoff S & Henikoff, J G, 1992, Proc. Natl. Acad. Sci. USA 89: 10915-10919 using a gap opening penalty of 10 and a gap extension penalty of 0.1, so that the highest order match obtained between two sequences where at least 50% of the total length of one of the two sequences is involved in the alignment.

The terms “UDCA intermediate”, “UDCA precursor”, and their grammatical equivalents are used interchangeably and refer to any substrate that can be used to produce UDCA. This includes substrates that are far removed from UDCA itself, such as sugar, desmosterol, and cholesterol. The term also expressly encompasses 7-alpha-hydroxycholesterol; 7α-hydroxy-4-cholesten-3-one; 7α-hydroxy-5β-cholestan-3-one; 5β-cholestane-3α,7α-diol; (25R)-3α,7α-dihydroxy-5β-cholestanoic acid; (25R)-3α,7α-dihydroxy-5β-cholestanoyl-CoA; (25S)-3α,7α-dihydroxy-5β-cholestanoyl-CoA; (24E)-3α,7α-dihydroxy-5β-cholest-24-enoyl-CoA; 3α,7α-dihydroxy-24-oxo-5β-cholestanoyl-CoA; 3α,7α-dihydroxy-5β-cholan-24-oyl-CoA; 3α-hydroxy-7-oxo-5β-cholan-24-oyl-CoA; 3α,7β-dihydroxy-5β-cholan-24-oyl-CoA; 7α,12α-dihydroxy-4-cholesten-3-one; 7α,12α-dihydroxy-5β-cholestan-3-one; 5β-cholestane-3α,7α,12α-triol; (25R)-3α,7α,12α-trihydroxy-5β-cholestan-26-oic acid; (25R)-3α,7α,12α-trihydroxy-5β-cholestanoyl-CoA; (25S)-3α,7α,12α-trihydroxy-5β-cholestanoyl-CoA; (24E)-3α,7α,12α-trihydroxy-5β-cholest-24-enoyl-CoA; 3α,7α,12α-trihydroxy-24-oxo-5β-cholestanoyl-CoA; 3α,7α,12α-trihydroxy-5β-cholan-24-oyl-CoA; and cholic acid.

As will be apparent to those of skill in the art upon reading this disclosure, each of the individual embodiments described and illustrated herein has discrete components and features, which can be readily separated from or combined with the features of any of the other several cases without departing from the scope or spirit of the present invention. Any recited method can be carried out in the order of events recited or in any other order that is logically possible.

Unless defined otherwise herein, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

Biosynthetic Pathway

The present invention relates in part to biosynthetic pathways that produce UDCA or a UDCA precursor. UDCA, also known as “ursodeoxycholic acid” or “ursodiol” is a secondary bile acid with a molecular formula C₂₄H₄₀O₄, a molar mass of 392.56 g/mol, and a CAS number of 128-13-2.

In certain embodiments, the pathway involves the conversion of 3α,7α(-dihydroxy-5β-cholanoic acid, also known as chenodeoxycholic acid or CDCA, to UDCA.

In certain embodiments, the pathway involves the conversion of the Co-A form of CDCA to UDCA. The Co-A form of CDCA is 3α,7α(-dihydroxy-5β-cholan-24-oyl-CoA, which is also known as Chenodeoxycholoyl-CoA or CDC-CoA.

In certain embodiments, the conversion of CDC-CoA to UDCA involves at least one of the following reactions: conversion of CDC-CoA to 3α-hydroxy-7-oxo-5β-cholan-24-oyl-CoA; conversion of 3α-hydroxy-7-oxo-5β-cholan-24-oyl-CoA to 3α,7β-dihydroxy-5β-cholan-24-oyl-CoA; and/or conversion of 3α,7β-dihydroxy-5β-cholan-24-oyl-CoA to UDCA.

In certain embodiments, the pathway involves the conversion of cholesterol to CDCA or CDC-CoA.

In certain embodiments, the conversion of cholesterol to CDC-CoA involves at least one of the following reactions: conversion of cholesterol to 7-alpha-hydroxycholesterol; conversion of 7-alpha-hydroxycholesterol to 7α-hydroxy-4-cholesten-3-one; conversion of 7α-hydroxy-4-cholesten-3-one to 7α-hydroxy-5β-cholestan-3-one; conversion of 7α-hydroxy-5β-cholestan-3-one to 5β-cholestane-3α,7α-diol; conversion of 5β-cholestane-3α,7α-diol to (25R)-3α,7α-dihydroxy-5β-cholestanoic acid; conversion of (25R)-3α,7α-dihydroxy-5β-cholestanoic acid to (25R)-3α,7α-dihydroxy-5β-cholestanoyl-CoA; conversion of (25R)-3α,7α-dihydroxy-5β-cholestanoyl-CoA to (25S)-3α,7α-dihydroxy-5β-cholestanoyl-CoA; conversion of (25S)-3α,7α-dihydroxy-5β-cholestanoyl-CoA to (24E)-3α,7α-dihydroxy-5β-cholest-24-enoyl-CoA; conversion of (24E)-3α,7α-dihydroxy-5β-cholest-24-enoyl-CoA to 3α,7α(-dihydroxy-24-oxo-5β-cholestanoyl-CoA; and/or conversion of 3α,7α(-dihydroxy-24-oxo-5β-cholestanoyl-CoA to CDC-CoA.

In certain embodiments, the pathway involves the conversion of cholesterol to cholic acid. Cholic acid can be chemically converted to UDCA.

In certain embodiments, the conversion of cholesterol to cholic acid may involve at least one of the following reactions: conversion of cholesterol to 7-alpha-hydroxycholesterol; the conversion of 7-alpha-hydroxycholesterol to 7α-hydroxy-4-cholesten-3-one; conversion of 7α-hydroxy-4-cholesten-3-one to 7α,12α-dihydroxy-4-cholesten-3-one; conversion of 7α,12α-dihydroxy-4-cholesten-3-one to 7α,12α-dihydroxy-5β-cholestan-3-one; conversion of 7α,12α-dihydroxy-5β-cholestan-3-one to 5β-cholestane-3α,7α,12α-triol; conversion of 5β-cholestane-3α,7α,12α-triol to (25R)-3α,7α,12α-trihydroxy-5β-cholestan-26-oic acid; conversion of (25R)-3α,7α,12α-trihydroxy-5β-cholestan-26-oic acid to (25R)-3α,7α,12α-trihydroxy-5β-cholestanoyl-CoA; conversion of (25R)-3α,7α,12α-trihydroxy-5β-cholestanoyl-CoA to (25S)-3α,7α,12α-trihydroxy-5β-cholestanoyl-CoA; conversion of (25S)-3α,7α,12α-trihydroxy-5β-cholestanoyl-CoA to (24E)-3α,7α,12α-trihydroxy-5β-cholest-24-enoyl-CoA; conversion of (24E)-3α,7α,12α-trihydroxy-5β-cholest-24-enoyl-CoA to 3α,7α,12α-trihydroxy-24-oxo-5β-cholestanoyl-CoA; conversion of 3α,7α,12α-trihydroxy-24-oxo-5β-cholestanoyl-CoA to 3α,7α,12α-trihydroxy-5β-cholan-24-oyl-CoA; and conversion of 3α,7α,12α-trihydroxy-5β-cholan-24-oyl-CoA to cholic acid.

In certain embodiments, the pathway involves the conversion of cholesta-5,7,24-trienol to cholesterol. The conversion of cholesta-5,7,24-trienol to cholesterol may involve the conversion of cholesta-5,7,24-trienol to desmosterol and/or the conversion of desmosterol to cholesterol. Cholesta-5,7,24-trienol is produced naturally from sugar by yeast.

Enzymes

Each of the aforementioned reactions and/or conversions may be catalyzed by an enzyme. For example:

7-dehydrocholesterol reductase (gene name: DHCR7) catalyzes the conversion of cholesta-5,7,24-trienol to desmosterol. DHCR7 can comprise an amino acid sequence of any one of SEQ ID NOs: 1, 3, 5, 7, 9, or 11, or an amino acid sequence substantially identical to any of the aforementioned sequences. DHCR7 can be encoded by a polynucleotide comprising a nucleic acid sequence of any one of SEQ ID NOs: 2, 4, 6, 8, 10, or 12, or a nucleic acid sequence substantially identical to any of the aforementioned sequences.

24-dehydrocholesterol reductase (gene name: DHCR24) catalyzes the conversion of desmosterol to cholesterol. DHCR24 can comprise an amino acid sequence of any one of SEQ ID NOs: 13, 17, 21, 25, 29, 33, 37, 41, 43, 45, or 47, or an amino acid sequence substantially identical to any of the aforementioned sequences. DHCR24 can be encoded by a polynucleotide comprising a nucleic acid sequence of any one of SEQ ID NOs: 14, 15, 16, 18, 19, 20, 22, 23, 24, 26, 27, 28, 30, 31, 32, 34, 35, 36, 38, 39, 40, 42, 44, 46, or 48, or a nucleic acid sequence substantially identical to any of the aforementioned sequences.

Cytochrome p450 family 7 subfamily A member 1 (abbreviation and gene name: CYP7A1) catalyzes the conversion of cholesterol to 7-alpha-hydroxycholesterol. CYP7A1 can comprise an amino acid sequence of any one of SEQ ID NOs: 49, 51, 53, 55, 57, 59, 61, 63, 65, 67, 69, 71, 73, 75, 77, or 79, or an amino acid sequence substantially identical to any of the aforementioned sequences. CYP7A1 can be encoded by a polynucleotide comprising a nucleic acid sequence of any one of SEQ ID NOs: 50, 52, 54, 56, 58, 60, 62, 64, 66, 68, 70, 72, 74, 76, 78, or 80, or a nucleic acid sequence substantially identical to any of the aforementioned sequences.

3 beta-hydroxysteroid dehydrogenase type 7 (abbreviation and gene name: HSD3B7) catalyzes the conversion of 7-alpha-hydroxycholesterol to 7α-hydroxy-4-cholesten-3-one. HSD3B7 can comprise an amino acid sequence of any one of SEQ ID NOs: 81, 83, 85, or 87, or an amino acid sequence substantially identical to any of the aforementioned sequences. HSD3B7 can be encoded by a polynucleotide comprising a nucleic acid sequence of any one of SEQ ID NOs: 82, 84, 86, or 88, or a nucleic acid sequence substantially identical to any of the aforementioned sequences.

Cytochrome p450 family 8 subfamily B member 1 (abbreviation and gene name: CYP8B1) catalyzes the conversion of 7α-hydroxy-4-cholesten-3-one to 7α,12α-dihydroxy-4-cholesten-3-one. CYP8B1 can comprise an amino acid sequence of any one of SEQ ID NOs: 265, 267, 269, 271, 273, 275, or 277, or an amino acid sequence substantially identical to any of the aforementioned sequences. CYP8B1 can be encoded by a polynucleotide comprising a nucleic acid sequence of any one of SEQ ID NOs: 266, 268, 270, 272, 274, 276, or 278, or a nucleic acid sequence substantially identical to any of the aforementioned sequences.

3-oxo-5-beta(β)-steroid 4-dehydrogenase also known as aldo-keto reductase family 1 member D1 (abbreviation and gene name: AKR1D1) catalyzes the conversion of 7α-hydroxy-4-cholesten-3-one to 7α-hydroxy-5β-cholestan-3-one. AKR1D1 also catalyzes the conversion of 7α,12α-dihydroxy-4-cholesten-3-one to 7α,12α-dihydroxy-5β-cholestan-3-one. AKR1D1 can comprise an amino acid sequence of any one of SEQ ID NOs: 89, 91, 93, or 95, or an amino acid sequence substantially identical to any of the aforementioned sequences. AKR1D1 can be encoded by a polynucleotide comprising a nucleic acid sequence of any one of SEQ ID NOs: 90, 92, 94, or 96, or a nucleic acid sequence substantially identical to any of the aforementioned sequences.

Aldo-keto reductase family 1 member C4 (abbreviation and gene name: AKR1C4) catalyzes the conversion of 7α-hydroxy-5β-cholestan-3-one to 5β-cholestane-3α,7α-diol. AKR1C4 also catalyzes the conversion of 7α,12α-dihydroxy-5β-cholestan-3-one to 5β-cholestane-3α,7α,12α-triol, AKR1C4 can comprise an amino acid sequence of any one of SEQ ID NOs: 99, 101, 103, 105, 107, 109, 111, 113, 115, 117, 119, or 121, or an amino acid sequence substantially identical to any of the aforementioned sequences. AKR1C4 can be encoded by a polynucleotide comprising a nucleic acid sequence of any one of SEQ ID NOs: 100, 102, 104, 106, 108, 110, 112, 114, 116, 118, 120, or 122, or a nucleic acid sequence substantially identical to any of the aforementioned sequences.

Cytochrome p450 family 27 subfamily A member 1 (abbreviation and gene name: CYP27A1), also known as sterol 27-hydroxylase, catalyzes the conversion of 5β-cholestane-3α,7α-diol to (25R)-3α,7α-dihydroxy-5β-cholestanoic acid. CYP27A1 also catalyzes the conversion of 5β-cholestane-3α7α,12α-triol to (25R)-3α,7α,12α-trihydroxy-5β-cholestan-26-oic acid. CYP27A1 can comprise an amino acid sequence of any one of SEQ ID NOs: 123, 125, 127, 129, 131, 133, 135, or 137, or an amino acid sequence substantially identical to any of the aforementioned sequences. CYP27A1 can be encoded by a polynucleotide comprising a nucleic acid sequence of any one of SEQ ID NOs: 124, 126, 128, 130, 132, 134, 136, or 138, or a nucleic acid sequence substantially identical to any of the aforementioned sequences.

Solute carrier family 27 member 5 (abbreviation and gene name: SLC27A5) or its yeast homologue FAT1, catalyzes the conversion of (25R)-3α,7α-dihydroxy-5β-cholestanoic acid to (25R)-3α,7α-dihydroxy-5β-cholestanoyl-CoA. SLC27A5 and FAT1 also catalyze the conversion of (25R)-3α,7α,12α-trihydroxy-5β-cholestan-26-oic acid to (25R)-3α,7α,12α-trihydroxy-5β-cholestanoyl-CoA. SLC27A5 can comprise an amino acid sequence of SEQ ID NOs: 139 or 141, or an amino acid sequence substantially identical to any of the aforementioned sequences. SLC27A5 can be encoded by a polynucleotide comprising a nucleic acid sequence of SEQ ID NOs: 140 or 142, or a nucleic acid sequence substantially identical to either of the aforementioned sequences. FAT1 can comprise an amino acid sequence of SEQ ID NO: 143, or an amino acid sequence substantially identical therewith. FAT1 can be encoded by a polynucleotide comprising a nucleic acid sequence of SEQ ID NO: 144, or a nucleic acid sequence substantially identical therewith.

Alpha-methylacyl-CoA racemase (abbreviation and gene name: AMACR) catalyzes the conversion of (25R)-3α,7α-dihydroxy-5β-cholestanoyl-CoA to (25S)-3α,7α-dihydroxy-5β-cholestanoyl-CoA. AMACR also catalyzes the conversion of (25R)-3α,7α,12α-trihydroxy-5β-cholestanoyl-CoA to (255)-3α,7α,12α-trihydroxy-5β-cholestanoyl-CoA. AMACR can comprise an amino acid sequence of any one of SEQ ID NOs: 145, 147, 149, 151, 153, 155, or 157, or an amino acid sequence substantially identical to any of the aforementioned sequences. AMACR can be encoded by a polynucleotide comprising a nucleic acid sequence of any one of SEQ ID NOs: 146, 148, 150, 152, 154, 156, or 158, or a nucleic acid sequence substantially identical to any of the aforementioned sequences.

Acyl-CoA oxidase 2 (abbreviation and gene name: ACOX2) or its yeast homologue PDX1 catalyze the conversion of (25S)-3α,7α-dihydroxy-5β-cholestanoyl-CoA to (24E)-3α,7α-dihydroxy-5β-cholest-24-enoyl-CoA. ACOX2 and PDX1 also catalyze the conversion of (255)-3α,7α,12α-trihydroxy-5β-cholestanoyl-CoA to (24E)-3α,7α,12α-trihydroxy-5β-cholest-24-enoyl-CoA. ACOX2 can comprise an amino acid sequence of any one of SEQ ID NOs: 159, 161, 163, 165, 167, 169, 171, or 173, or an amino acid sequence substantially identical to any of the aforementioned sequences. ACOX2 can be encoded by a polynucleotide comprising a nucleic acid sequence of any one of SEQ ID NOs: 160, 162, 164, 166, 168, 170, 172, or 174, or a nucleic acid sequence substantially identical to any of the aforementioned sequences. PDX1 can comprise an amino acid sequence of SEQ ID NO: 175, or an amino acid sequence substantially identical therewith. PDX1 can be encoded by a polynucleotide comprising a nucleic acid sequence of SEQ ID NO: 176, or a nucleic acid sequence substantially identical therewith.

Hydroxysteroid 17-beta dehydrogenase 4 (abbreviation and gene name: HSD17B4) or its yeast homologue FOX2 catalyze the conversion of (24E)-3α,7α-dihydroxy-5β-cholest-24-enoyl-CoA to 3α,7α-dihydroxy-24-oxo-5β-cholestanoyl-CoA. HSD17B4 and FOX 2 also catalyze the conversion of (24E)-3α,7α,12α-trihydroxy-5β-cholest-24-enoyl-CoA to 3α,7α,12α-trihydroxy-24-oxo-5β-cholestanoyl-CoA. HSD17B4 and FOX2 can comprise an amino acid sequence of any one of SEQ ID NOs: 177, 179, 181, 183, 185, 187, 189, or 191, or an amino acid sequence substantially identical to any of the aforementioned sequences. HSD17B4 can be encoded by a polynucleotide comprising a nucleic acid sequence of any one of SEQ ID NOs: 178, 180, 182, 184, 186, 188, 190, or 192, or a nucleic acid sequence substantially identical to any of the aforementioned sequences. FOX2 can comprise an amino acid sequence of SEQ ID NO: 193, or an amino acid sequence substantially identical therewith. FOX2 can be encoded by a polynucleotide comprising a nucleic acid sequence of SEQ ID NO: 194, or a nucleic acid sequence substantially identical therewith.

Sterol carrier protein 2 (abbreviation and gene name: SCP2) or its yeast homologues POT1 or ERG10 catalyze the conversion of 3α,7α-dihydroxy-24-oxo-5β-cholestanoyl-CoA to CDC-CoA. SCP2, POT1, and ERG10 also catalyze the conversion of 3α,7α,12α-trihydroxy-24-oxo-5β-cholestanoyl-CoA to 3α,7α,12α-trihydroxy-5β-cholan-24-oyl-CoA. SCP2 can comprise an amino acid sequence of any one of SEQ ID NOs: 195, 197, 199, or 201, or an amino acid sequence substantially identical to any of the aforementioned sequences. SCP2 can be encoded by a polynucleotide comprising a nucleic acid sequence of any one of SEQ ID NOs: 196, 198, 200, or 202, or a nucleic acid sequence substantially identical to any of the aforementioned sequences. POT1 can comprise an amino acid sequence of SEQ ID NO: 203, or an amino acid sequence substantially identical therewith. POT1 can be encoded by a polynucleotide comprising a nucleic acid sequence of SEQ ID NO: 204, or a polynucleotide having a nucleotide sequence substantially identical therewith. ERG10 can comprise an amino acid sequence of SEQ ID NO: 205, or an amino acid sequence substantially identical therewith. ERG10 can be encoded by a polynucleotide comprising a nucleic acid sequence of SEQ ID NO: 206, or a nucleic acid sequence substantially identical therewith.

7alpha-hydroxysteroid dehydrogenase (abbreviation and gene name: 7α-HSD) catalyzes the conversion of CDC-CoA to 3α-hydroxy-7-oxo-5β-cholan-24-oyl-CoA. 7α-HSD can comprise an amino acid sequence of any one of SEQ ID NOs: 207, 209, 211, or 213, or an amino acid sequence substantially identical to any of the aforementioned sequences. 7α-HSD can be encoded by a polynucleotide comprising a nucleic acid sequence of any one of SEQ ID NOs: 208, 210, 212, or 214, or a nucleic acid sequence substantially identical to any of the aforementioned sequences.

7beta-hydroxysteroid dehydrogenase (abbreviation and gene name: 7β-HSD) catalyzes the conversion of 3α-hydroxy-7-oxo-5β-cholan-24-oyl-CoA to 3α,7β-dihydroxy-5β-cholan-24-oyl-CoA. 7β-HSD can comprise an amino acid sequence of any one of SEQ ID NOs: 215, 217, 219, or 221, or an amino acid sequence substantially identical to any of the aforementioned sequences. 7β-HSD can be encoded by a polynucleotide comprising a nucleic acid sequence of any one of SEQ ID NOs: 216, 218, 220, or 222, or a nucleic acid sequence substantially identical to any of the aforementioned sequences.

Choloyl-CoA hydrolase catalyzes the conversion of 3α,7β-dihydroxy-5β-cholan-24-oyl-CoA to UDCA. Choloyl-CoA hydrolase also catalyzes the conversion of 3α,7α, 12α-trihydroxy-5β-cholan-24-oyl-CoA to cholic acid. Choloyl-CoA hydrolase can comprise an amino acid sequence of any one of SEQ ID NOs: 223, 225, 227, or 229, or an amino acid sequence substantially identical to any of the aforementioned sequences. Choloyl-CoA hydrolase can be encoded by a polynucleotide comprising a nucleic acid sequence of any one of SEQ ID NOs: 224, 226, 228, or 230, or a nucleic acid sequence substantially identical to any of the aforementioned sequences. In some cases, the choloyl-CoA hydrolase has an EC number of 3.12.27.

Aldo-Keto Reductase Family 1 Member C9 (abbreviation and gene name: AKR1C9) can comprise an amino acid of SEQ ID NO: 97, or an amino acid sequence substantially identical thereto. AKR1C9 can be encoded by a polynucleotide comprising a nucleic acid sequence of SEQ ID NO: 98, or a nucleic acid sequence substantially identical therewith.

Bile acid-CoA:amino acid N-acyltransferase (abbreviation: N-acyltransferase) catalyzes the conversion of 3α,7β-dihydroxy-5β-cholan-24-oyl-CoA to glyco-ursodeoxycholic acid (glycol-UDCA). N-acyltransferase can comprise an amino acid sequence of any one of SEQ ID NOs: 232, 234, 236, or 238, or an amino acid sequence substantially identical to any of the aforementioned sequences. Choloyl-CoA hydrolase can be encoded by a polynucleotide comprising a nucleic acid sequence of any one of SEQ ID NOs: 224, 226, 228, or 232, 234, 236, or 238, or a nucleic acid sequence substantially identical to any of the aforementioned sequences.

The present invention also contemplates the use of fragments of any of the aforementioned enzymes. In certain embodiments, the fragment is one that retains the desired biological activity of the respective full-length enzyme. Such fragments will be referred to herein as “biologically-active” fragments.

A biologically-active fragment of DHCR7 for use in the present invention may be one that retains the ability to catalyze the conversion of cholesta-5,7,24-trienol to desmosterol. A biologically-active fragment of DHCR24 for use in the present invention may be one that retains the ability to catalyze the conversion of desmosterol to cholesterol. A biologically-active fragment of CYP7A1 for use in the present invention may be one that retains the ability to catalyze the conversion of cholesterol to 7-alpha-hydroxycholesterol. A biologically-active fragment of HSD3B7 for use in the present invention may be one that retains the ability to catalyze the conversion of 7-alpha-hydroxycholesterol to 7α-hydroxy-4-cholesten-3-one. A biologically-active fragment of CYP8B1 for use in the present invention may be one that retains the ability to catalyze the conversion of 7α-hydroxy-4-cholesten-3-one to 7α,12α-dihydroxy-4-cholesten-3-one. A biologically-active fragment of AKR1D1 for use in the present invention may be one that retains the ability to catalyze the conversion of 7α-hydroxy-4-cholesten-3-one to 7α-hydroxy-5β-cholestan-3-one and/or the conversion of 7α,12α-dihydroxy-4-cholesten-3-one to 7α,12α-dihydroxy-5β-cholestan-3-one. A biologically-active fragment of AKR1C4 for use in the present invention may be one that retains the ability to catalyze the conversion of 7α-hydroxy-5β-cholestan-3-one to 5β-cholestane-3α,7α-diol and/or or the conversion of 7α,12α-dihydroxy-5β-cholestan-3-one to 5β-cholestane-3α,7α,12α-triol. A biologically-active fragment of CYP27A1 for use in the present invention may be one that retains the ability to catalyze the conversion of 5β-cholestane-3α,7α-diol to (25R)-3α,7α-dihydroxy-5β-cholestanoic acid and/or the conversion of 5β-cholestane-3α7α,12α-triol to (25R)-3α,7α,12α-trihydroxy-5β-cholestan-26-oic acid. A biologically-active fragment of SLC27A5 or FAT 1 for use in the present invention may be one that retains the ability to catalyze the conversion of (25R)-3α,7α-dihydroxy-5β-cholestanoic acid to (25R)-3α,7α-dihydroxy-5β-cholestanoyl-CoA and/or the conversion of (25R)-3α,7α,12α-trihydroxy-5β-cholestan-26-oic acid to (25R)-3α,7α,12α-trihydroxy-5β-cholestanoyl-CoA. A biologically-active fragment of AMACR for use in the present invention may be one that retains the ability to catalyze the conversion of (25R)-3α,7α-dihydroxy-5β-cholestanoyl-CoA to (25S)-3α,7α-dihydroxy-5β-cholestanoyl-CoA and/or the conversion of (25R)-3α,7α,12α-trihydroxy-5β-cholestanoyl-CoA to (255)-3α,7α,12α-trihydroxy-5β-cholestanoyl-CoA. A biologically-active fragment of ACOX2 or POX1 for use in the present invention may be one that retains the ability to catalyze the conversion of (25S)-3α,7α-dihydroxy-5β-cholestanoyl-CoA to (24E)-3α,7α-dihydroxy-5β-cholest-24-enoyl-CoA and/or the conversion of (25S)-3α,7α,12α-trihydroxy-5β-cholestanoyl-CoA to (24E)-3α,7α,12α-trihydroxy-5β-cholest-24-enoyl-CoA. A biologically-active fragment of HSD17B4 or FOX2 for use in the present invention may be one that retains the ability to catalyze the conversion of (24E)-3α,7α-dihydroxy-5β-cholest-24-enoyl-CoA to 3α,7α-dihydroxy-24-oxo-5β-cholestanoyl-CoA and/or the conversion of (24E)-3α,7α,12α-trihydroxy-5β-cholest-24-enoyl-CoA to 3α,7α,12α-trihydroxy-24-oxo-5β-cholestanoyl-CoA. A biologically-active fragment of SCP2, POT1, or ERG10 for use in the present invention may be one that retains the ability to catalyze the conversion of 3α,7α-dihydroxy-24-oxo-5β-cholestanoyl-CoA to CDC-CoA and/or the conversion of 3α,7α,12α-trihydroxy-24-oxo-5β-cholestanoyl-CoA to 3α,7α,12α-trihydroxy-5β-cholan-24-oyl-CoA. A biologically-active fragment of 7α-HSD for use in the present invention may be one that retains the ability to catalyze the conversion of CDC-CoA to 3α-hydroxy-7-oxo-5β-cholan-24-oyl-CoA. A biologically-active fragment of 7β-HSD for use in the present invention may be one that retains the ability to catalyze the conversion of 3α-hydroxy-7-oxo-5β-cholan-24-oyl-CoA to 3α,7β-dihydroxy-5β-cholan-24-oyl-CoA. A biologically-active fragment of choloyl-CoA hydrolase for use in the present invention may be one that retains the ability to catalyze the conversion of 3α,7β-dihydroxy-5β-cholan-24-oyl-CoA to UDCA and/or 3α,7α,12α-trihydroxy-5β-cholan-24-oyl-CoA to cholic acid. A biologically-active fragment of N-acyltransferase for use in the present invention may be one that retains the ability to catalyze the conversion of 3α,7β-dihydroxy-5β-cholan-24-oyl-CoA to glycol-UDCA.

Genetically-Modified Cell

The present invention relates in part to a genetically-modified cell capable of producing UDCA, cholic acid and/or another UDCA precursor. The genetically-modified cell can be used to ferment UDCA, cholic acid and/or UDCA precursor in a fermentation tank.

In certain embodiments, the cell comprises at least one heterologous enzyme, or biologically-active fragment thereof, involved in a biosynthetic pathway that produces UDCA, cholic acid, and/or another UDCA precursor, for example a pathway as described previously. In certain embodiments, the cell comprises two or more, three or more, four or more, five or more, six or more, seven or more, eight or more, nine or more, ten or more, eleven or more, twelve or more, thirteen or more, fourteen or more, fifteen or more, or sixteen or more such enzymes and/or biologically-active fragments thereof. In certain such embodiments, the enzymes or biologically-active fragments thereof are operably connected along a biosynthetic pathway. The heterologous enzyme may, for example, be DHCR7, DHCR24, CYP7A1, HSD3B7, CYP8B1, AKR1D1, AKR1C4, CYP27A1, SLC27A5, AMACR, ACOX2, HSD17B4, SCP2, 7α-HSD, 7β-HSD, choloyl-CoA hydrolase, AKR1C9, or N-acyltransferase. The cell may comprise an enzyme having an amino acid sequence as described previously for the respective enzyme.

In an embodiment wherein the cell comprises a heterologous DHCR7, the enzyme may comprise an amino acid sequence of any one of SEQ ID NOs: 1, 3, 5, 7, 9, or 11, or an amino acid sequence substantially identical to any of the aforementioned sequences.

In an embodiment wherein the cell comprises a heterologous DHCR24, the enzyme may comprise an amino acid sequence of any one of SEQ ID NOs: 13, 17, 21, 25, 29, 33, 37, 41, 43, 45, or 47, or an amino acid sequence substantially identical to any of the aforementioned sequences.

In an embodiment wherein the cell comprises a heterologous CYP7A1, the enzyme may comprise an amino acid sequence of any one of SEQ ID NOs: 49, 51, 53, 55, 57, 59, 61, 63, 65, 67, 69, 71, 73, 75, 77, or 79, or an amino acid sequence substantially identical to any of the aforementioned sequences.

In an embodiment wherein the cell comprises a heterologous HSD3B7, the enzyme may comprise an amino acid sequence of any one of SEQ ID NOs: 81, 83, 85, or 87, or an amino acid sequence substantially identical to any of the aforementioned sequences.

In an embodiment wherein the cell comprises a heterologous AKR1D1, the enzyme may comprise an amino acid sequence of any one of SEQ ID NOs: 89, 91, 93, or 95, or an amino acid sequence substantially identical to any of the aforementioned sequences.

In an embodiment wherein the cell comprises a heterologous CYP8B1, the enzyme may comprise an amino acid sequence of any one of SEQ ID NOs: 265, 267, 269, 271, 273, 275, or 277, or an amino acid sequence substantially identical to any of the aforementioned sequences.

In an embodiment wherein the cell comprises a heterologous AKR1C4, the enzyme may comprise an amino acid sequence of any one of SEQ ID NOs: 99, 101, 103, 105, 107, 109, 111, 113, 115, 117, 119, or 121, or an amino acid sequence substantially identical to any of the aforementioned sequences.

In an embodiment wherein the cell comprises a heterologous CYP27A1, the enzyme may comprise an amino acid sequence of any one of SEQ ID NOs: 123, 125, 127, 129, 131, 133, 135, or 137, or an amino acid sequence substantially identical to any of the aforementioned sequences.

In an embodiment wherein the cell comprises a heterologous SLC27A5, the enzyme may comprise an amino acid sequence of SEQ ID NOs: 139 or 141, or an amino acid sequence substantially identical to any of the aforementioned sequences.

In an embodiment wherein the cell comprises a heterologous FAT1, the enzyme may comprise an amino acid sequence of SEQ ID NO: 143, or an amino acid sequence substantially identical therewith.

In an embodiment wherein the cell comprises a heterologous AMACR, the enzyme may comprise an amino acid sequence of any one of SEQ ID NOs: 145, 147, 149, 151, 153, 155, or 157, or an amino acid sequence substantially identical to any of the aforementioned sequences.

In an embodiment wherein the cell comprises a heterologous ACOX2, the enzyme may comprise an amino acid sequence of any one of SEQ ID NOs: 159, 161, 163, 165, 167, 169, 171, or 173, or an amino acid sequence substantially identical to any of the aforementioned sequences.

In an embodiment wherein the cell comprises a heterologous FOX1, the enzyme may comprise an amino acid sequence of SEQ ID NO: 175, or an amino acid sequence substantially identical therewith.

In an embodiment wherein the cell comprises a heterologous HSD17B4, the enzyme may comprise an amino acid sequence of any one of SEQ ID NOs: 177, 179, 181, 183, 185, 187, 189, or 191, or an amino acid sequence substantially identical to any of the aforementioned sequences.

In an embodiment wherein the cell comprises a heterologous FOX2, the enzyme may comprise an amino acid sequence of SEQ ID NO: 193, or an amino acid sequence substantially identical therewith.

In an embodiment wherein the cell comprises a heterologous SCP2, the enzyme may comprise an amino acid sequence of any one of SEQ ID NOs: 195, 197, 199, or 201, or an amino acid sequence substantially identical to any of the aforementioned sequences.

In an embodiment wherein the cell comprises a heterologous POT1, the enzyme may comprise an amino acid sequence of SEQ ID NO: 203, or an amino acid sequence substantially identical therewith.

In an embodiment wherein the cell comprises a heterologous ERG10, the enzyme may comprise an amino acid sequence SEQ ID NO: 205, or an amino acid sequence substantially identical therewith.

In an embodiment wherein the cell comprises a heterologous 7α-HSD, the enzyme may comprise an amino acid sequence of any one of SEQ ID NOs: 207, 209, 211, or 213, or an amino acid sequence substantially identical to any of the aforementioned sequences.

In an embodiment wherein the cell comprises a heterologous 7β-HSD, the enzyme may comprise an amino acid sequence of any one of SEQ ID NOs: 215, 217, 219, or 221, or an amino acid sequence substantially identical to any of the aforementioned sequences.

In an embodiment wherein the cell comprises a heterologous choloyl-CoA hydrolase, the enzyme may comprise an amino acid sequence of any one of SEQ ID NOs: 223, 225, 227, or 229, or an amino acid sequence substantially identical to any of the aforementioned sequences.

In an embodiment wherein the cell comprises a heterologous AKR1C9, the enzyme may comprise an amino acid sequence of SEQ ID NO: 97, or an amino acid sequence substantially identical to any of the aforementioned sequences.

In an embodiment wherein the cell comprises a heterologous N-acyltransferase, the enzyme may comprise an amino acid sequence of any one of SEQ ID NOs: 232, 234, 236, or 238, or an amino acid sequence substantially identical to any of the aforementioned sequences.

In certain embodiments, the cell comprises at least one heterologous polynucleotide encoding an enzyme, or biologically-active fragment thereof, involved in a biosynthetic pathway that produces UDCA, cholic acid, and/or another UDCA precursor, for example a pathway as described previously. In certain embodiments, the cell comprises two or more, three or more, four or more, five or more, six or more, seven or more, eight or more, nine or more, ten or more, eleven or more, twelve or more, thirteen or more, fourteen or more, fifteen or more, or sixteen or more such polynucleotides. The heterologous polynucleotide may, for example, encode DHCR7, DHCR24, CYP7A1, HSD3B7, CYP8B1, AKR1D1, AKR1C4, CYP27A1, SLC27A5, AMACR, ACOX2, HSD17B4, SCP2, 7α-HSD, 7β-HSD, and/or choloyl-CoA hydrolase, and/or a biologically-active fragment of such an enzyme. In certain such embodiments, the enzymes and/or biologically-active fragments thereof are operably connected along a biosynthetic pathway.

In an embodiment wherein the cell comprises a heterologous polynucleotide encoding DHCR7, the polynucleotide may comprise a nucleic acid sequence of any one of SEQ ID NOs: 2, 4, 6, 8, 10, or 12, or a nucleic acid sequence substantially identical to any of the aforementioned sequences.

In an embodiment wherein the cell comprises a heterologous polynucleotide encoding DHCR24, the polynucleotide may comprise a nucleic acid sequence of any one of SEQ ID NOs: 14, 15, 16, 18, 19, 20, 22, 23, 24, 26, 27, 28, 30, 31, 32, 34, 35, 36, 38, 39, 40, 42, 44, 46, or 48, or a nucleic acid sequence substantially identical to any of the aforementioned sequences.

In an embodiment wherein the cell comprises a heterologous polynucleotide encoding CYP7A1, the polynucleotide may comprise a nucleic acid sequence of any one of SEQ ID NOs: 50, 52, 54, 56, 58, 60, 62, 64, 66, 68, 70, 72, 74, 76, 78, or 80, or a nucleic acid sequence substantially identical to any of the aforementioned sequences.

In an embodiment wherein the cell comprises a heterologous polynucleotide encoding HSD3B7, the polynucleotide may comprise a nucleic acid sequence of any one of SEQ ID NOs: 82, 84, 86, or 88, or a nucleic acid sequence substantially identical to any of the aforementioned sequences.

In an embodiment wherein the cell comprises a heterologous polynucleotide encoding CYP8B1, the polynucleotide may comprise a nucleic acid sequence of any one of SEQ ID NOs: 266, 268, 270, 272, 274, 276, or 278, or a nucleic acid sequence substantially identical to any of the aforementioned sequences.

In an embodiment wherein the cell comprises a heterologous polynucleotide encoding AKR1D1, the polynucleotide may comprise a nucleic acid sequence of any one of SEQ ID NOs: 90, 92, 94, or 96, or a nucleic acid sequence substantially identical to any of the aforementioned sequences.

In an embodiment wherein the cell comprises a heterologous polynucleotide encoding AKR1C4, the polynucleotide may comprise a nucleic acid sequence of any one of SEQ ID NOs: 100, 102, 104, 106, 108, 110, 112, 114, 116, 118, 120, or 122, or a nucleic acid sequence substantially identical to any of the aforementioned sequences.

In an embodiment wherein the cell comprises a heterologous polynucleotide encoding CYP27A1, the polynucleotide may comprise a nucleic acid sequence of any one of SEQ ID NOs: 124, 126, 128, 130, 132, 134, 136, or 138, or a nucleic acid sequence substantially identical to any of the aforementioned sequences.

In an embodiment wherein the cell comprises a heterologous polynucleotide encoding SLC27A5, the polynucleotide may comprise a nucleic acid sequence of SEQ ID NOs: 140 or 142, or a nucleic acid sequence substantially identical to either of the aforementioned sequences.

In an embodiment wherein the cell comprises a heterologous polynucleotide encoding FAT1, the polynucleotide may comprise a nucleic acid sequence of SEQ ID NO: 144, or a nucleic acid sequence substantially identical therewith.

In an embodiment wherein the cell comprises a heterologous polynucleotide encoding AMACR, the polynucleotide may comprise a nucleic acid sequence of any one of SEQ ID NOs: 146, 148, 150, 152, 154, 156, or 158, or a nucleic acid sequence substantially identical to any of the aforementioned sequences.

In an embodiment wherein the cell comprises a heterologous polynucleotide encoding ACOX2, the polynucleotide may comprise a nucleic acid sequence of any one of SEQ ID NOs: 160, 162, 164, 166, 168, 170, 172, or 174, or a nucleic acid sequence substantially identical to any of the aforementioned sequences.

In an embodiment wherein the cell comprises a heterologous polynucleotide encoding FOX1, the polynucleotide may comprise a nucleic acid sequence of SEQ ID NO: 176, or a nucleic acid sequence substantially identical therewith.

In an embodiment wherein the cell comprises a heterologous polynucleotide encoding HSD17B4, the polynucleotide may comprise a nucleic acid sequence of any one of SEQ ID NOs: 178, 180, 182, 184, 186, 188, 190, or 192, or a nucleic acid sequence substantially identical to any of the aforementioned sequences.

In an embodiment wherein the cell comprises a heterologous polynucleotide encoding FOX2, the polynucleotide may comprise a nucleic acid sequence of SEQ ID NO: 194, or a nucleic acid sequence substantially identical therewith.

In an embodiment wherein the cell comprises a heterologous polynucleotide encoding SCP2, the polynucleotide may comprise a nucleic acid sequence of any one of SEQ ID NOs: 196, 198, 200, or 202, or a nucleic acid sequence substantially identical to any of the aforementioned sequences.

In an embodiment wherein the cell comprises a heterologous polynucleotide encoding POT1, the polynucleotide may comprise a nucleic acid sequence of SEQ ID NO: 204, or a nucleic acid sequence substantially identical therewith.

In an embodiment wherein the cell comprises a heterologous polynucleotide encoding ERG10, the polynucleotide may comprise a nucleic acid sequence of SEQ ID NO: 206, or a nucleic acid sequence substantially identical therewith.

In an embodiment wherein the cell comprises a heterologous polynucleotide encoding 7α-HSD, the polynucleotide may comprise a nucleic acid sequence of any one of SEQ ID NOs: 208, 210, 212, or 214, or a nucleic acid sequence substantially identical to any of the aforementioned sequences.

In an embodiment wherein the cell comprises a heterologous polynucleotide encoding 7β-HSD, the polynucleotide may comprise a nucleic acid sequence of any one of SEQ ID NOs: 216, 218, 220, or 222, or a nucleic acid sequence substantially identical to any of the aforementioned sequences.

In an embodiment wherein the cell comprises a heterologous polynucleotide encoding choloyl-CoA hydrolase, the polynucleotide may comprise a nucleic acid sequence of any one of SEQ ID NOs: 224, 226, 228, or 230, or a nucleic acid sequence substantially identical to any of the aforementioned sequences.

In an embodiment wherein the cell comprises a heterologous polynucleotide encoding AKR1C9, the polynucleotide may comprise a nucleic acid sequence of SEQ ID NO: 98, or a nucleic acid sequence substantially identical therewith.

In an embodiment wherein the cell comprises a heterologous polynucleotide encoding N-acyltransferase, the polynucleotide may comprise a nucleic acid sequence of SEQ ID NOs: 232, 234, 236, or 238, or a polynucleotide having a nucleotide sequence substantially identical to any of the aforementioned sequences.

In certain embodiments, the polynucleotide encodes two or more, three or more, four or more, five or more, six or more, seven or more, eight or more, nine or more, ten or more, eleven or more, twelve or more, thirteen or more, fourteen or more, fifteen or more, or sixteen or more such enzymes and/or biologically-active fragments thereof. In certain such embodiments, the enzymes or biologically-active fragments thereof are operably connected along a biosynthetic pathway.

In certain embodiments, the cell comprises at least one heterologous enzyme, or biologically-active fragment thereof, capable of catalyzing at least one of the following conversions: cholesta-5,7,24-trienol to desmosterol; desmosterol to cholesterol; cholesterol to 7-alpha-hydroxycholesterol; 7-alpha-hydroxycholesterol to 7α-hydroxy-4-cholesten-3-one; 7α-hydroxy-4-cholesten-3-one to 7α-hydroxy-5β-cholestan-3-one; 7α-hydroxy-5β-cholestan-3-one to 5β-cholestane-3α,7α-diol; 5β-cholestane-3α,7α-diol to (25R)-3α,7α-dihydroxy-5β-cholestanoic acid; (25R)-3α,7α-dihydroxy-5β-cholestanoic acid to (25R)-3α,7α-dihydroxy-5β-cholestanoyl-CoA; (25R)-3α,7α-dihydroxy-5β-cholestanoyl-CoA to (25S)-3α,7α-dihydroxy-5β-cholestanoyl-CoA; (25S)-3α,7α-dihydroxy-5β-cholestanoyl-CoA to (24E)-3α,7α-dihydroxy-5β-cholest-24-enoyl-CoA; (24E)-3α,7α-dihydroxy-5β-cholest-24-enoyl-CoA to 3α,7α-dihydroxy-24-oxo-5β-cholestanoyl-CoA; and 3α,7α-dihydroxy-24-oxo-5β-cholestanoyl-CoA to CDC-CoA. In certain embodiments, the cell comprises at least one heterologous polynucleotide encoding such an enzyme or biologically-active fragment thereof.

In certain embodiments, the cell comprises at least one heterologous enzyme, or biologically-active fragment thereof, that catalyzes at least one of the following conversions: cholesterol to 7-alpha-hydroxycholesterol; 7-alpha-hydroxycholesterol to 7α-hydroxy-4-cholesten-3-one; 7α-hydroxy-4-cholesten-3-one to 7α,12α-dihydroxy-4-cholesten-3-one; 7α,12α-dihydroxy-4-cholesten-3-one to 7α,12α-dihydroxy-5β-cholestan-3-one; 7α,12α-dihydroxy-5β-cholestan-3-one to 5β-cholestane-3α,7α,12α-triol; 5β-cholestane-3α,7α,12α-triol to (25R)-3α,7α,12α-trihydroxy-5β-cholestan-26-oic acid; (25R)-3α,7α,12α-trihydroxy-5β-cholestan-26-oic acid to (25R)-3α,7α,12α-trihydroxy-5β-cholestanoyl-CoA; (25R)-3α,7α,12α-trihydroxy-5β-cholestanoyl-CoA to (25S)-3α,7α,12α-trihydroxy-5β-cholestanoyl-CoA; (25S)-3α,7α,12α-trihydroxy-5β-cholestanoyl-CoA to (24E)-3α,7α,12α-trihydroxy-5β-cholest-24-enoyl-CoA; (24E)-3α,7α,12α-trihydroxy-5β-cholest-24-enoyl-CoA to 3α,7α,12α-trihydroxy-24-oxo-5β-cholestanoyl-CoA; 3α,7α,12α-trihydroxy-24-oxo-5β-cholestanoyl-CoA to 3α,7α,12α-trihydroxy-5β-cholan-24-oyl-CoA; and 3α,7α,12α-trihydroxy-5β-cholan-24-oyl-CoA to cholic acid. In certain embodiments, the cell comprises at least one heterologous polynucleotide encoding such an enzyme or biologically-active fragment thereof.

In certain embodiments, the cell comprises at least one heterologous enzyme, or biologically-active fragment thereof, that catalyzes at least one of the following conversions: CDC-CoA to 3α-hydroxy-7-oxo-5β-cholan-24-oyl-CoA; 3α-hydroxy-7-oxo-5β-cholan-24-oyl-CoA to 3α,7β-dihydroxy-5β-cholan-24-oyl-CoA; and 3α,7β-dihydroxy-5β-cholan-24-oyl-CoA to UDCA. In certain embodiments, the cell comprises at least one heterologous polynucleotide encoding such an enzyme or biologically-active fragment thereof.

Additionally, a hydrolase, or biologically-active fragment thereof, can act on the CoA forms of the desired products to make a free acid form of the desired products. In some cases, the free acid form of the desired products can include (25R)-3α,7α-dihydroxy-5β-cholestanoic acid; (25S)-3α,7α(-dihydroxy-5β-cholestanoic acid; (24E)-3α,7α-dihydroxy-5β-cholest-24-enoic acid; 3α,7α-dihydroxy-24-oxo-5β-cholestanoic acid; 3α,7α(-dihydroxy-5β-cholanoic acid (chenodeoxycholic acid; CDCA); 3α-hydroxy-7-oxo-5β-cholanoic acid (nutriacholic acid; NCA); 3α,7β-dihydroxy-5β-cholanoic acid (ursodeoxycholic acid; UDCA); (25R)-3α,7α,12α-trihydroxy-5β-cholestan-26-oic acid; (25S)-3α,7α,12α-trihydroxy-5β-cholestanoic acid; (24E)-3α,7α,12α-trihydroxy-5β-cholest-24-enoic acid; 3α,7α,12α-trihydroxy-24-oxo-5β-cholestanoic acid; cholic acid; or any combination thereof.

The cell may also be engineered to express heterologous enzymes, or biologically-active fragments thereof, to improve the production of UDCA or UDCA precursor(s).

In certain embodiments, adrenodoxin reductase (ADR), or a biologically-active fragment thereof, may be used to improve the production of UDCA or UDCA precursor(s). In such an embodiment, the genetically-modified cell may comprise at least one heterologous ADR enzyme or a biologically-active fragment of such an enzyme. In certain embodiments, the enzyme comprises an amino acid sequence of SEQ ID NO: 239, or an amino acid sequence substantially identical therewith. In certain embodiments, the cell may comprise at least one heterologous polynucleotide encoding ADR or a biologically-active fragment thereof. The polynucleotide may comprise a nucleic acid sequence of SEQ ID NO: 240, or a polynucleotide having a nucleotide sequence substantially identical therewith.

In certain embodiments, adrenodoxin (ADX), or a biologically-active fragment thereof, may be used to improve the production of UDCA or UDCA precursor(s). In such an embodiment, the genetically-modified cell may comprise at least one heterologous ADX enzyme or a biologically-active fragment of such an enzyme. In certain embodiments, the enzyme comprises an amino acid sequence of any one of SEQ ID NO: 241, 243, 245, 247, 249, 251, 253, 255, 257, 259, or 261, or an amino acid sequence substantially identical to any of the aforementioned sequences. In certain embodiments, the cell may comprise at least one heterologous polynucleotide encoding ADX or a biologically-active fragment thereof. The polynucleotide may comprise a nucleic acid sequence of any one of SEQ ID NOs: 242, 244, 246, 248, 250, 252, 254, 256, 258, 260, or 262, or a polynucleotide having a nucleotide sequence substantially identical to any of the aforementioned sequences.

In certain embodiments, a truncated HMG, or a biologically-active fragment thereof may be used to improve the production of UDCA or UDCA precursor(s). In such an embodiment, the genetically-modified cell may comprise at least one truncated HMG or a biologically-active fragment of such an enzyme. In certain embodiments, the enzyme comprises an amino acid sequence of SEQ ID NO: 263, or an amino acid sequence substantially identical therewith. In certain embodiments, the cell may comprise at least one heterologous polynucleotide encoding truncated HMG or a biologically-active fragment thereof. The polynucleotide may comprise a nucleic acid sequence of SEQ ID NO: 264, or a polynucleotide having a nucleotide sequence substantially identical therewith.

In certain embodiments, the amino acid sequence of the enzyme is optimized to correspond to amino acid usage within the host cell.

In certain embodiments, the nucleic acid sequence of the polynucleotide is codon optimized for usage within the host cell.

The enzymes disclosed throughout can be from a microorganism. For example, the enzymes can be from bacteria, archaea, fungi, protozoa, algae, and/or viruses. The enzymes can also come from an animal, such as mammals, e.g., Homo sapiens and Mus musculus, or from plants, such as Arabidopsis.

The enzymes or fragments thereof described throughout can also be in some cases fused or linked together. Any fragment linker can be used to link two or more of the enzymes or fragments thereof together. In some cases, the linker can be any random array of amino acid sequences.

In certain embodiments, the cell is a microorganism or part of one, or part of a plant, animal, or fungus. The microorganism may be yeast, algae, or bacterium. The microorganism may be prokaryotic or eukaryotic. In certain embodiments, the microorganism is a bacterium or a yeast. For example, the microorganism may be Saccharomyces cerevisiae, Yaffoniia lipolytica, or Escherichia coli, or any other cell disclosed throughout.

In certain embodiments, the microorganism is a yeast. Examples of yeast that may be used include those from the genus Saccharomyces. In certain embodiments, the yeast is of the species Saccharomyces cerevisiae.

Should the genetically-modified microorganism be a bacterium, the bacterium can be from the genus Escherichia, e.g., Escherichia coli.

In certain embodiments, the cell is not naturally capable of producing UDCA, cholic acid, and/or other UDCA precursors or produces the same in lower than desired quantities. By implementation of the genetic modification described herein, the cell may be modified such that the level of UDCA, cholic acid, and/or other UDCA precursors therein is higher relative to the level of UDCA, cholic acid, and/or other UDCA precursors in a corresponding unmodified cell.

In certain embodiments, the cell is naturally capable of catalyzing some, but not all, of the reactions necessary to produce UDCA, cholic acid, and/or other UDCA precursors. For example, the cell may be naturally capable of catalyzing some, but not all, of the conversions in the aforementioned biosynthetic pathways for producing UDCA, cholic acid, and/or other UDCA precursors.

In certain embodiments, the cell is naturally capable of producing a substrate that may be used to produce UDCA, cholic acid, and/or other UDCA precursors. However, the cell is not naturally capable of producing UDCA, cholic acid, and/or other UDCA precursors. In such embodiments, the genetic modification may serve to allow the cell to convert the substrate into UDCA, CDCA, CDC-CoA, cholic acid, or other UDCA precursors.

In certain embodiments, the genetically-modified cell is unable to produce a substrate that can be used to produce UDCA, cholic acid, and/or other UDCA precursors. In such embodiments, the substrate may be provided to the cell, for example as part of the cell's growth medium. The cell can then convert this substrate into UDCA, cholic acid, and/or other UDCA precursors.

In some cases, the genetically modified microorganism can make UDCA or a UDCA precursor, such as CDC-CoA or cholic acid, from one or more substrates.

Isolated Polynucleotides

The present invention relates in part to an isolated polynucleotide encoding an enzyme involved in a biosynthetic pathway that produces UDCA, cholic acid, and/or another UDCA precursor. In other words, the gene can be in a form that does not exist in nature, isolated from a chromosome. The isolated polynucleotide may encode at least one of the aforementioned enzymes and may comprise any one of the respective sequences encoding such an enzyme.

The isolated polynucleotides can be inserted into the genome of the cell/microorganism used. In some cases, the isolated polynucleotide is inserted into the genome at a specific locus, where the isolated polynucleotide can be expressed in sufficient amounts.

In certain embodiments, the isolated polynucleotide encodes at least one enzyme, or biologically-active fragment thereof, capable of catalyzing at least one of the following conversions: cholesta-5,7,24-trienol to desmosterol; desmosterol to cholesterol; cholesterol to 7-alpha-hydroxycholesterol; 7-alpha-hydroxycholesterol to 7α-hydroxy-4-cholesten-3-one; 7α-hydroxy-4-cholesten-3-one to 7α-hydroxy-5β-cholestan-3-one; 7α-hydroxy-5β-cholestan-3-one to 5β-cholestane-3α,7α-diol; 5β-cholestane-3α,7α-diol to (25R)-3α,7α-dihydroxy-5β-cholestanoic acid; (25R)-3α,7α-dihydroxy-5β-cholestanoic acid to (25R)-3α,7α-dihydroxy-5β-cholestanoyl-CoA; (25R)-3α,7α-dihydroxy-5β-cholestanoyl-CoA to (25S)-3α,7α-dihydroxy-5β-cholestanoyl-CoA; (25S)-3α,7α-dihydroxy-5β-cholestanoyl-CoA to (24E)-3α,7α-dihydroxy-5β-cholest-24-enoyl-CoA; (24E)-3α,7α-dihydroxy-5β-cholest-24-enoyl-CoA to 3α,7α-dihydroxy-24-oxo-5β-cholestanoyl-CoA; 3α,7α-dihydroxy-24-oxo-5β-cholestanoyl-CoA to CDC-CoA.

In certain embodiments, the isolated polynucleotide encodes at least one enzyme, or biologically-active fragment thereof, that catalyzes at least one of the following conversions: cholesterol to 7-alpha-hydroxycholesterol; 7-alpha-hydroxycholesterol to 7α-hydroxy-4-cholesten-3-one; 7α-hydroxy-4-cholesten-3-one to 7α,12α-dihydroxy-4-cholesten-3-one; 7α,12α-dihydroxy-4-cholesten-3-one to 7α,12α-dihydroxy-5β-cholestan-3-one; 7α,12α-dihydroxy-5β-cholestan-3-one to 5β-cholestane-3α,7α,12α-triol; 5β-cholestane-3α,7α,12α-triol to (25R)-3α,7α,12α-trihydroxy-5β-cholestan-26-oic acid; (25R)-3α,7α,12α-trihydroxy-5β-cholestan-26-oic acid to (25R)-3α,7α,12α-trihydroxy-5β-cholestanoyl-CoA; (25R)-3α,7α,12α-trihydroxy-5β-cholestanoyl-CoA to (25S)-3α,7α,12α-trihydroxy-5β-cholestanoyl-CoA; (25S)-3α,7α,12α-trihydroxy-5β-cholestanoyl-CoA to (24E)-3α,7α,12α-trihydroxy-5β-cholest-24-enoyl-CoA; (24E)-3α,7α,12α-trihydroxy-5β-cholest-24-enoyl-CoA to 3α,7α,12α-trihydroxy-24-oxo-5β-cholestanoyl-CoA; 3α,7α,12α-trihydroxy-24-oxo-5β-cholestanoyl-CoA to 3α,7α,12α-trihydroxy-5β-cholan-24-oyl-CoA; and 3α,7α,12α-trihydroxy-5β-cholan-24-oyl-CoA to cholic acid.

In certain embodiments, the isolated polynucleotide encodes at least one enzyme, or biologically-active fragment thereof, that catalyzes at least one of the following conversions: CDC-CoA to 3α-hydroxy-7-oxo-5β-cholan-24-oyl-CoA; 3α-hydroxy-7-oxo-5β-cholan-24-oyl-CoA to 3α,7α-dihydroxy-5β-cholan-24-oyl-CoA; and 3α,7β-dihydroxy-5β-cholan-24-oyl-CoA to UDCA.

In certain embodiments, the isolated polynucleotide encodes DHCR7, DHCR24, CYP7A1, HSD3B7, CYP8B1, AKR1D1, AKR1C4, CYP27A1, SLC27A5, AMACR, ACOX2, HSD17B4, SCP2, 7α-HSD, 7β-HSD, choloyl-CoA hydrolase, AKR1C9, and/or N-acyltransferase, and/or a biologically-active fragment of such an enzyme.

In an embodiment wherein the isolated polynucleotide encodes DHCR7, the isolated polynucleotide comprises a nucleic acid sequence of any one of SEQ ID NOs: 2, 4, 6, 8, 10, or 12, or a nucleic acid sequence substantially identical to any of the aforementioned sequences.

In an embodiment wherein the isolated polynucleotide encodes DHCR24, the isolated polynucleotide comprises a nucleic acid sequence of any one of SEQ ID NOs: 14, 15, 16, 18, 19, 20, 22, 23, 24, 26, 27, 28, 30, 31, 32, 34, 35, 36, 38, 39, 40, 42, 44, 46, or 48, or a nucleic acid sequence substantially identical to any of the aforementioned sequences.

In an embodiment wherein the isolated polynucleotide encodes CYP7A1, the isolated polynucleotide comprises a nucleic acid sequence of any one of SEQ ID NOs: 50, 52, 54, 56, 58, 60, 62, 64, 66, 68, 70, 72, 74, 76, 78, or 80, or a nucleic acid sequence substantially identical to any of the aforementioned sequences.

In an embodiment wherein the isolated polynucleotide encodes HSD3B7, the isolated polynucleotide comprises a nucleic acid sequence of any one of SEQ ID NOs: 82, 84, 86, or 88, or a nucleic acid sequence substantially identical to any of the aforementioned sequences.

In an embodiment wherein the isolated polynucleotide encodes CYP8B1, the isolated polynucleotide comprises a nucleic acid sequence of any one of SEQ ID NOs: 266, 268, 270, 272, 274, 276, or 278, or a nucleic acid sequence substantially identical to any of the aforementioned sequences.

In an embodiment wherein the isolated polynucleotide encodes AKR1D1, the isolated polynucleotide comprises a nucleic acid sequence of any one of SEQ ID NOs: 90, 92, 94, or 96, or a nucleic acid sequence substantially identical to any of the aforementioned sequences.

In an embodiment wherein the isolated polynucleotide encodes AKR1C4, the isolated polynucleotide comprises a nucleic acid sequence of any one of SEQ ID NOs: 100, 102, 104, 106, 108, 110, 112, 114, 116, 118, 120, or 122, or a nucleic acid sequence substantially identical to any of the aforementioned sequences.

In an embodiment wherein the isolated polynucleotide encodes CYP27A1, the isolated polynucleotide comprises a nucleic acid sequence of any one of SEQ ID NOs: 124, 126, 128, 130, 132, 134, 136, or 138, or a nucleic acid sequence substantially identical to any of the aforementioned sequences.

In an embodiment wherein the isolated polynucleotide encodes SLC27A5, the isolated polynucleotide comprises a nucleic acid sequence of SEQ ID NOs: 140 or 142, or a nucleic acid sequence substantially identical to either of the aforementioned sequences.

In an embodiment wherein the isolated polynucleotide encodes FAT1, the isolated polynucleotide comprises a nucleic acid sequence of SEQ ID NO: 144, or a nucleic acid sequence substantially identical therewith.

In an embodiment wherein the isolated polynucleotide encodes AMACR, the isolated polynucleotide comprises a nucleic acid sequence of any one of SEQ ID NOs: 146, 148, 150, 152, 154, 156, or 158, or a nucleic acid sequence substantially identical to any of the aforementioned sequences.

In an embodiment wherein the isolated polynucleotide encodes ACOX2, the isolated polynucleotide comprises a nucleic acid sequence of any one of SEQ ID NOs: 160, 162, 164, 166, 168, 170, 172, or 174, or a nucleic acid sequence substantially identical to any of the aforementioned sequences.

In an embodiment wherein the isolated polynucleotide encodes FOX1, the isolated polynucleotide comprises e a nucleic acid sequence of SEQ ID NO: 176, or a nucleic acid sequence substantially identical therewith.

In an embodiment wherein the isolated polynucleotide encodes HSD17B4, the isolated polynucleotide comprises a nucleic acid sequence of any one of SEQ ID NOs: 178, 180, 182, 184, 186, 188, 190, or 192, or a nucleic acid sequence substantially identical to any of the aforementioned sequences.

In an embodiment wherein the isolated polynucleotide encodes FOX2, the isolated polynucleotide comprises a nucleic acid sequence of SEQ ID NO: 194, or a nucleic acid sequence substantially identical therewith.

In an embodiment wherein the isolated polynucleotide encodes SCP2, the isolated polynucleotide comprises a nucleic acid sequence of any one of SEQ ID NOs: 196, 198, 200, or 202, or a nucleic acid sequence substantially identical to any of the aforementioned sequences.

In an embodiment wherein the isolated polynucleotide encodes POT1, the isolated polynucleotide comprises a nucleic acid sequence of SEQ ID NO: 204, or a nucleic acid sequence substantially identical therewith.

In an embodiment wherein the isolated polynucleotide encodes ERG10, the isolated polynucleotide comprises e a nucleic acid sequence of SEQ ID NO: 206, or a nucleic acid sequence substantially identical therewith.

In an embodiment wherein the isolated polynucleotide encodes 7α-HSD, the isolated polynucleotide comprises a nucleic acid sequence of any one of SEQ ID NOs: 208, 210, 212, or 214, or a nucleic acid sequence substantially identical to any of the aforementioned sequences.

In an embodiment wherein the isolated polynucleotide encodes 7β-HSD, the isolated polynucleotide comprises a nucleic acid sequence of any one of SEQ ID NOs: 216, 218, 220, or 222, or a nucleic acid sequence substantially identical to any of the aforementioned sequences.

In an embodiment wherein the isolated polynucleotide encodes choloyl-CoA hydrolase, the isolated polynucleotide comprises a nucleic acid sequence of any one of SEQ ID NOs: 224, 226, 228, or 230, or a nucleic acid sequence substantially identical to any of the aforementioned sequences.

In an embodiment wherein the isolated polynucleotide encodes AKR1C9, the isolated polynucleotide comprises a nucleic acid sequence of any one of SEQ ID NO: 98, or a nucleic acid sequence substantially identical therewith.

In an embodiment wherein the isolated polynucleotide encodes N-acyltransferase, the isolated polynucleotide comprises a nucleic acid sequence of any one of SEQ ID NOs: 232, 234, 236, or 238, or a polynucleotide having a nucleotide sequence substantially identical to any of the aforementioned sequences.

The isolated polynucleotide may also encode at least one enzyme that improves the production of UDCA, cholic acid, and/or other UDCA precursors, such as ADR, ADX, and/or a truncated HMG, and/or a biologically-active fragment of such an enzyme.

In an embodiment wherein the isolated polynucleotide encodes ADR, the isolated polynucleotide comprises a nucleic acid sequence of any one of SEQ ID NO: 240, or a polynucleotide having a nucleotide sequence substantially identical therewith.

In an embodiment wherein the isolated polynucleotide encodes ADX, the isolated polynucleotide comprises a nucleic acid sequence of any one of SEQ ID NOs: 242, 244, 246, 248, 250, 252, 254, 256, 258, 260, or 262, or a polynucleotide having a nucleotide sequence substantially identical to any of the aforementioned sequences.

In an embodiment wherein the isolated polynucleotide encodes truncated HMG, the isolated polynucleotide comprises a nucleic acid sequence of any one of SEQ ID NO: 264, or a polynucleotide having a nucleotide sequence substantially identical therewith.

Vectors

Since some of the enzymes and biologically-active fragments thereof described throughout are not native to some cells and microorganisms, expression vectors can be used to express the desired enzymes and/or fragments within most microorganisms and cells. The present invention thus also relates in part to a vector comprising a polynucleotide as described previously encoding an enzyme, or biologically-active fragment thereof, involved in a biosynthetic pathway that produces UDCA, cholic acid, and/or another UDCA precursor.

Vector constructs prepared for introduction into the host cells or microorganisms described throughout can typically, but not always, comprise a replication system (i.e. vector) recognized by the host. In some cases, the vector includes the intended polynucleotide fragment encoding the desired enzyme or fragment thereof and, optionally, transcription and translational initiation regulatory sequences operably linked to the polypeptide-encoding segment. Expression vectors can include, for example, an origin of replication or autonomously replicating sequence (ARS), expression control sequences, a promoter, an enhancer and necessary processing information sites, such as ribosome-binding sites, RNA splice sites, polyadenylation sites, transcriptional terminator sequences, mRNA stabilizing sequences, polynucleotides homologous to host chromosomal DNA, and/or a multiple cloning site. Signal peptides can also be included where appropriate, for example from secreted polypeptides of the same or related species, that allow the protein to cross and/or lodge in cell membranes or be secreted from the cell.

The expression vector may be introduced into the host cell stably or transiently into a host cell, using established techniques, including, but not limited to, electroporation, calcium phosphate precipitation, DEAE-dextran mediated transfection, liposome-mediated transfection, heat shock in the presence of lithium acetate, and the like. For stable transformation, a nucleic acid will generally further include a selectable marker, e.g., any of several well-known selectable markers such as neomycin resistance, ampicillin resistance, tetracycline resistance, chloramphenicol resistance, kanamycin resistance, and the like. In some embodiments, the nucleic acid with which the host cell is genetically modified is an expression vector that includes a nucleic acid comprising a nucleotide sequence that encodes a gene product, e.g., an enzyme, a transcription factor, etc.

Suitable expression vectors include, but are not limited to, baculovirus vectors, bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, herpes simplex virus, and the like), P1-based artificial chromosomes, yeast plasmids, yeast artificial chromosomes, and any other vectors specific for specific hosts of interest (such as yeast). Thus, for example, a nucleic acid encoding a gene product(s) is included in any one of a variety of expression vectors for expressing the gene product(s). Such vectors include chromosomal, nonchromosomal and synthetic DNA sequences.

In some cases, the promoter used in the vector can be sensitive to a chemical substance. For example, in the presence of the chemical substance, the promoter is either activated or deactivated. In some cases, the chemical substance can be a sugar such as glucose or galactose. In some cases, the chemical substance can be copper. In some cases, the chemical substance can be a rare earth metal. In some cases, the rare earth metal can be lanthanum or cerium. In some cases, the rare earth metal can be praseodymium or neodymium.

The vectors can be constructed using standard methods (see, e.g., Sambrook et al., Molecular Biology: A Laboratory Manual, Cold Spring Harbor, N.Y. 1989; and Ausubel, et al., Current Protocols in Molecular Biology, Greene Publishing, Co. N.Y, 1995).

Manipulation of polynucleotides that encode the enzymes or biologically-active fragments thereof disclosed throughout is typically carried out in recombinant vectors. Vectors that can be employed include yeast plasmids, bacterial plasmids, bacteriophage, artificial chromosomes, episomal vectors and gene expression vectors. Vectors can be selected to accommodate a polynucleotide encoding a protein of a desired size. Following production of a selected vector, a suitable host cell (e.g., the microorganisms described herein) is transfected or transformed with the vector. Each vector contains various functional components, which generally include a cloning site and an origin of replication. In some cases, the vector comprises at least one selectable marker gene. A vector can additionally possess one or more of the following elements: an enhancer, promoter, a transcription termination sequence and/or other signal sequences. Such sequence elements can be optimized for the selected host species. Such sequence elements can be positioned in the vicinity of the cloning site, such that they are operatively linked to the gene encoding a preselected enzyme.

Vectors, including cloning and expression vectors, can contain polynucleotides that enable the vector to replicate in one or more selected microorganisms. For example, the sequence can be one that enables the vector to replicate independently of the host chromosomal DNA and can include origins of replication or autonomously replicating sequences. Such sequences are well known for a variety of bacteria, yeast and viruses. For example, the origin of replication from the plasmid pBR322 is suitable for most gram-negative bacteria, the origin of replication for 2 micron plasmid is suitable for yeast, and various viral origins of replication (e.g., SV40, adenovirus) are useful for cloning vectors.

A cloning or expression vector can contain a selection gene, also referred to as a selectable marker. This gene encodes a protein necessary for the survival or growth of transformed microorganisms in a selective culture medium. Microorganisms not transformed with the vector containing the selection gene will therefore not survive in the culture medium. Typical selection genes encode proteins that confer resistance to antibiotics and other toxins, e.g. ampicillin, neomycin, methotrexate, hygromycin, kanamyxin, thiostrepton, apramycin or tetracycline, complement auxotrophic deficiencies, or supply critical nutrients not available in the growth media.

The replication of vectors can be performed in E. coli. An example of an E. coli-selectable marker is the β-lactamase gene, which confers resistance to the antibiotic ampicillin. These selectable markers can be obtained from E. coli plasmids, such as pBR322 or a pUC plasmid such as pUC18 or pUC19, or pUC119.

In an embodiment wherein the vector comprises a polynucleotide encoding DHCR7, the isolated vector may comprise a nucleic acid sequence of any one of SEQ ID NOs: 2, 4, 6, 8, 10, or 12, or a nucleic acid sequence substantially identical to any of the aforementioned sequences.

In an embodiment wherein the vector comprises a polynucleotide encoding DHCR24, the isolated vector may comprise nucleic acid sequence of any one of SEQ ID NOs: 14, 15, 16, 18, 19, 20, 22, 23, 24, 26, 27, 28, 30, 31, 32, 34, 35, 36, 38, 39, 40, 42, 44, 46, or 48, or a nucleic acid sequence substantially identical to any of the aforementioned sequences.

In an embodiment wherein the vector comprises a polynucleotide encoding CYP7A1, the isolated vector may comprise a nucleic acid sequence of any one of SEQ ID NOs: 50, 52, 54, 56, 58, 60, 62, 64, 66, 68, 70, 72, 74, 76, 78, or 80, or a nucleic acid sequence substantially identical to any of the aforementioned sequences.

In an embodiment wherein the vector comprises a polynucleotide encoding HSD3B7, the isolated vector may comprise a nucleic acid sequence of any one of SEQ ID NOs: 82, 84, 86, or 88, or a nucleic acid sequence substantially identical to any of the aforementioned sequences.

In an embodiment wherein the vector comprises a polynucleotide encoding CYP8B1, the isolated vector may comprise a nucleic acid sequence of any one of SEQ ID NOs: 50, 52, 54, 56, 58, 60, 62, 64, 66, 68, 70, 72, 74, 76, 78, or 80, or a nucleic acid sequence substantially identical to any of the aforementioned sequences.

In an embodiment wherein the vector comprises a polynucleotide encoding AKR1D1, the isolated vector may comprise a nucleic acid sequence of any one of SEQ ID NOs: 90, 92, 94, or 96, or a nucleic acid sequence substantially identical to any of the aforementioned sequences.

In an embodiment wherein the vector comprises a polynucleotide encoding AKR1C4, the isolated vector may comprise a nucleic acid sequence of any one of SEQ ID NOs: 100, 102, 104, 106, 108, 110, 112, 114, 116, 118, 120, or 122, or a nucleic acid sequence substantially identical to any of the aforementioned sequences.

In an embodiment wherein the vector comprises a polynucleotide encoding CYP27A1, the isolated vector may comprise a nucleic acid sequence of any one of SEQ ID NOs: 124, 126, 128, 130, 132, 134, 136, or 138, or a nucleic acid sequence substantially identical to any of the aforementioned sequences.

In an embodiment wherein the vector comprises a polynucleotide encoding SLC27A5, the isolated vector may comprise a nucleic acid sequence of SEQ ID NOs: 140 or 142, or a nucleic acid sequence substantially identical to either of the aforementioned sequences.

In an embodiment wherein the vector comprises a polynucleotide encoding FAT1, the isolated vector may comprise a nucleic acid sequence of SEQ ID NO: 144, or a nucleic acid sequence substantially identical therewith.

In an embodiment wherein the vector comprises a polynucleotide encoding AMACR, the isolated vector may comprise a nucleic acid sequence of SEQ ID NOs: 146, 148, 150, 152, 154, 156, or 158, or a nucleic acid sequence substantially identical to any of the aforementioned sequences.

In an embodiment wherein the vector comprises a polynucleotide encoding ACOX2, the isolated vector may comprise a nucleic acid sequence of SEQ ID NOs: 160, 162, 164, 166, 168, 170, 172, or 174, or a nucleic acid sequence substantially identical to any of the aforementioned sequences.

In an embodiment wherein the vector comprises a polynucleotide encoding FOX1, the isolated vector may comprise a nucleic acid sequence of SEQ ID NO: 176, or a nucleic acid sequence substantially identical therewith.

In an embodiment wherein the vector comprises a polynucleotide encoding HSD17B4, the isolated vector may comprise a nucleic acid sequence of SEQ ID NOs: 178, 180, 182, 184, 186, 188, 190, or 192, or a nucleic acid sequence substantially identical to any of the aforementioned sequences.

In an embodiment wherein the vector comprises a polynucleotide encoding FOX2, the isolated vector may comprise a nucleic acid sequence of SEQ ID NO: 194, or a nucleic acid sequence substantially identical therewith.

In an embodiment wherein the vector comprises a polynucleotide encoding SCP2, the isolated vector may comprise a nucleic acid sequence of any one of SEQ ID NOs: 196, 198, 200, or 202, or a nucleic acid sequence substantially identical to any of the aforementioned sequences.

In an embodiment wherein the vector comprises a polynucleotide encoding POT1, the isolated vector may comprise a nucleic acid sequence of SEQ ID NO: 204, or a nucleic acid sequence substantially identical therewith.

In an embodiment wherein the vector comprises a polynucleotide encoding ERG10, the isolated vector may comprise a nucleic acid sequence of SEQ ID NO: 206, or a nucleic acid sequence substantially identical therewith.

In an embodiment wherein the vector comprises a polynucleotide encoding 7α-HSD, the isolated vector may comprise a nucleic acid sequence of SEQ ID NOs: 208, 210, 212, or 214, or a nucleic acid sequence substantially identical to any of the aforementioned sequences.

In an embodiment wherein the vector comprises a polynucleotide encoding 7β-HSD, the isolated vector may comprise a nucleic acid sequence of SEQ ID NOs: 216, 218, 220, or 222, or a nucleic acid sequence substantially identical to any of the aforementioned sequences.

In an embodiment wherein the vector comprises a polynucleotide encoding choloyl-CoA hydrolase, the isolated vector may comprise a nucleic acid sequence of SEQ ID NOs: 224, 226, 228, or 230, or a nucleic acid sequence substantially identical to any of the aforementioned sequences.

In an embodiment wherein the vector comprises a polynucleotide encoding AKR1C9, the isolated vector may comprise a nucleic acid sequence of SEQ ID NO: 98, or a nucleic acid sequence substantially identical therewith.

In an embodiment wherein the vector comprises a polynucleotide encoding N-acyltransferase, the isolated vector may comprise a nucleic acid sequence of SEQ ID NOs: 232, 234, 236, or 238, or a polynucleotide having a nucleotide sequence substantially identical to any of the aforementioned sequences.

In an embodiment wherein the vector comprises a polynucleotide encoding ADR, the isolated vector may comprise a nucleic acid sequence of SEQ ID NO: 240, or a polynucleotide having a nucleotide sequence substantially identical therewith.

In an embodiment wherein the vector comprises a polynucleotide encoding ADX, the isolated vector may comprise a nucleic acid sequence of SEQ ID NOs: 242, 244, 246, 248, 250, 252, 254, 256, 258, 260, or 262, or a polynucleotide having a nucleotide sequence substantially identical to any of the aforementioned sequences.

In an embodiment wherein the vector comprises a polynucleotide encoding truncated HMG, the isolated vector may comprise a nucleic acid sequence of SEQ ID NO: 264, or a polynucleotide having a nucleotide sequence substantially identical therewith.

Promoters

Vectors can contain a promoter that is recognized by the host microorganism. The promoter can be operably linked to a coding sequence of interest. Such a promoter can be inducible, repressible, or constitutive. Polynucleotides are operably linked when the polynucleotides are in a relationship permitting them to function in their intended manner.

Different promoters can be used to drive the expression of the genes. For example, if temporary gene expression (i e. , non-constitutively expressed) is desired, expression can be driven by inducible or repressible promoters. The molecular switch can in some cases comprise these inducible or repressible promoters.

In some cases, the desired gene is expressed temporarily. In other words, the desired gene is not constitutively expressed. The expression of the desired gene can be driven by an inducible or repressible promoter, which functions as a molecular switch. Examples of inducible or repressible switches include, but are not limited to, those promoters inducible or repressible by: (a) sugars such as glucose, galactose, arabinose, and lactose (or non-metabolizable analogs, e.g., isopropyl β-D-1-thiogalactopyranoside (IPTG)); (b) metals such as copper or calcium (or rare earth metals such as lanthanum or cerium); (c) temperature; (d) Nitrogen-source; (e) oxygen; (f) cell state (growth or stationary); (g) metabolites such as phosphate; (h) CRISPRi; (i) jun; (j) fos, (k) metallothionein and/or (1) heat shock.

Inducible or repressible switches that can be particularly useful are switches that are responsive to sugars, metal ions, and rare earth metals. For example, promoters that are sensitive to arabinose, glucose, and/or galactose can be used as such switches. In some cases, such switches can be used to drive expression of one or more genes. For example, in the presence such a sugar, the arabinose or glucose to galactose switch can turn on the expression of a desired gene.

In particular embodiments, the switch is a GAL1 or GAL10 promoter. Such promoters are strongly repressed in the presence of glucose and depletion of glucose removes repression but does not necessarily trigger induction. However, in the presence of galactose, expression is strongly induced. To further achieve strong levels of expression, the GAL80 gene, which encodes a transcriptional repressor involved in transcriptional regulation mediated by galactose, may be knocked-out.

Metal ion switches of particular usefulness in this invention are copper sensitive switches. In some cases, the copper switch can be an inducible switch that can be used to “turn on” expression of one or more genes when copper is present in the environment. In the absence of copper in the media, the desired gene set or vector is not highly expressed.

Other useful switches can be rare earth metal switches, such as lanthanum sensitive switches (also simply known as a lanthanum switch). In some cases, the lanthanum switch can be a repressible switch that can be used to repress expression of one or more genes, until the repressor is removed (e.g., in this case lanthanum), after which the genes are “turned-on”. For example, in the presence the rare earth metal lanthanum, the desired gene set or vector can be “turned-off.” The expression of the genes is induced by either removing the lanthanum from the media or diluting the lanthanum in the media to levels where its repressible effects are reduced, minimized, or eliminated. Other rare earth metal switches can be used, such as those disclosed throughout.

Constitutively expressed promoters can also be used in the vector systems herein. For example, the expression of one or more desired genes can be controlled by constitutively active promoters. Examples of such promoters include but are not limited to pPGK1, pTDH3, pENO1, pTEF1, pHIS4, pUGA1, pADH1, pADH2, pGAL1, pGAL10, pGAL1/10, pXoxF, pMxaF, and p.Bba.J23111.

Promoters suitable for use with prokaryotic hosts can include, for example, the a-lactamase and lactose promoter systems, alkaline phosphatase, the tryptophan (trp) promoter system, the erythromycin promoter, apramycin promoter, hygromycin promoter, methylenomycin promoter and hybrid promoters such as the tac promoter. Promoters for use in bacterial systems will also generally contain a Shine-Dalgarno sequence operably linked to the coding sequence.

Promoters suitable for use with eukaryotic hosts can include, for example, galactose promoters, copper promoters, tetracycline promoters, glucose repressible promoters such as pGAL1 and pGAL10, low glucose induced promoters such as pADH2 and pHXT7, and high glucose induced promoters such as pHXT3. Such promoters will also generally contain a Kozak sequence operably linked to the coding sequence.

Generally, a strong promoter can be employed to provide for high level transcription and expression of the desired product. For example, promoters that can be used include but are not limited to pMxaF, pTDH3, pPGK1, pENO2, pTEF1, pTEF2, pADH1, pCCW12, pGAL1 and pGAL10. In some cases, a mutation can increase the strength of the promoter and therefore result in elevated levels of expression.

In some cases however, a weaker promoter is desired. For example, this is the case where too much expression of a certain gene results in a detrimental effect (e.g., the killing of cells). A weak promoter can be used, for example pPHO84, pPFK1, pCDC19, pBAD, pPHO84, pPFK1, pCLN1, pCYC1, pUGA1, pRAT1, and pPFK12. However, in some cases, a weaker promoter can be made by mutation. For example, the pmxaF promoters can be mutated to be weaker.

One or more promoters of a transcription unit can be an inducible promoter. For example, a GFP can be expressed from a constitutive promoter while an inducible promoter is used to drive transcription of a gene coding for one or more enzymes as disclosed herein and/or the amplifiable selectable marker.

Some vectors can contain sequences that facilitate the propagation of the vector in the host cell. Thus, the vectors can have other components such as an origin of replication (e.g., a polynucleotide that enables the vector to replicate in one or more selected microorganisms), antibiotic resistance genes for selection, and/or an amber stop codon that can permit translation to read through the codon. Additional selectable gene(s) can also be incorporated. Generally, in cloning vectors, the origin of replication is one that enables the vector to replicate independently of the host chromosomal DNA, and includes origins of replication or autonomously replicating sequences. Such sequences can include the ColE1 origin of replication in bacteria, a 2 micron origin of replication in yeast, or other known sequences.

The genes described throughout can all have a promoter driving their expression. The methods described herein, e.g., genome editing, can be used to edit the polynucleotide of the promoters or used to inhibit the effectiveness of the promoters. Inhibition can be done by blocking the transcription machinery (e.g., transcription factors) from binding to the promoter or by altering the promoter in such a way that the transcription machinery no longer recognizes the promoter sequence.

Methods of Making a Genetically-Modified Cell

The present invention relates in part to a method for making the previously-described genetically-modified cell. The method comprises contacting a cell with at least one heterologous polynucleotide encoding an enzyme involved in a biosynthetic pathway that produces UDCA, cholic acid, and/or another UDCA precursor, or a biologically-active fragment of such an enzyme. Such polynucleotides are as described previously. The method may further comprise growing the cell so that the heterologous polynucleotide is inserted into the cell.

In certain embodiments, the cell is contacted with at least two such heterologous polynucleotides. In such embodiments, the heterologous polynucleotides may encode enzymes and/or fragments thereof that are operably connected along the pathway.

In certain embodiments, the heterologous polynucleotide(s) are comprised in a vector, as discussed previously.

The genetically-modified cells and microorganisms disclosed throughout can be made in a variety of ways. For example, the cell or microorganism may be modified (e.g., genetically-engineered) by any method to comprise and/or express one or more polynucleotides encoding enzymes and/or fragments thereof in a pathway. For example, one or more of any of the genes discussed throughout can be inserted into a cell or microorganism. The genes can be inserted by an expression vector. The genes can also be under the control of one or more different/same promoters or the one or more genes can be under the control of a switch, such as an inducible or repressible promoter, e.g., an arabinose switch, glucose to galactose switch, isopropyl 13-D-1-thiogalactopyranoside (IPTG) switch, copper switch, or a rare earth metal switch. The genes can also be stably integrated into the genome of the microorganism. In some cases, the genes can be expressed in an episomal vector.

An exemplary method of making a genetically modified cell or microorganism disclosed herein is contacting (or transforming) a cell/microorganism with a polynucleotide encoding at least one of the enzymes described previously, or a fragment thereof. The polynucleotides that are inserted into the microorganism can be heterologous to the cell/microorganism itself. For example, if the microorganism is a yeast, the inserted polynucleotides can be from a bacterium, or a different species of yeast. Further, the polynucleotides can be endogenously part of the genome of the cell/microorganism.

In some embodiments, the method of the invention further comprises isolating the UDCA, cholic acid, and/or other UDCA precursor from the host microorganism and/or from the culture medium.

In certain embodiments, a UDCA precursor produced using a genetically-modified cell/microorganism is contacted with an unmodified cell that converts the UDCA precursor into another UDCA precursor or UDCA.

In certain embodiments, the UDCA precursor produced is not a substrate for further reactions.

In general, the genetically-modified host cell/microorganism is cultured in a suitable medium, optionally supplemented with one or more additional agents, such as an inducer (e.g., where one or more nucleotide sequences encoding a gene product is under the control of an inducible promoter). In some embodiments, the culture medium is overlaid with an organic solvent, e.g., dodecane, forming an organic layer. In such cases, the UDCA, cholic acid, and/or other UDCA precursor produced by the genetically-modified host cell/microorganism may partition into the organic layer, from which it can be purified. In some embodiments, where one or more gene product-encoding nucleotide sequence is operably linked to an inducible promoter, an inducer is added to the culture medium; and, after a suitable time, the UDCA, cholic acid, and/or other UDCA precursor is isolated from the organic layer overlaid on the culture medium.

In some embodiments, the UDCA, cholic acid, and/or other UDCA precursor is separated from other products which may be present in the organic layer. Such separation may be achieved using, e.g., standard chromatographic techniques.

In some embodiments, the UDCA, cholic acid, and/or other UDCA precursor is substantially pure.

Techniques for Genetic Modification

The cells/microorganisms disclosed herein can be genetically engineered by using classic microbiological techniques. Some of such techniques are generally disclosed, for example, in Sambrook et al., 1989, Molecular Cloning: A Laboratory Manual, Cold Spring Harbor Labs Press.

The genetically modified cells/microorganisms disclosed herein can include a polynucleotide that has been inserted, deleted or modified (i .e. , mutated; e.g., by insertion, deletion, substitution, and/or inversion of nucleotides), in such a manner that such modifications provide the desired effect of expression (e.g., over-expression) of one or more enzymes as provided herein within the cell/microorganism. Genetic modifications that result in an increase in gene expression or function can be referred to as amplification, overproduction, overexpression, activation, enhancement, addition, or up-regulation of a gene. Addition of a gene to increase gene expression can include maintaining the gene(s) on replicating plasmids or integrating the cloned gene(s) into the genome of the production cell/microorganism. Furthermore, increasing the expression of desired genes can include operatively linking the cloned gene(s) to native or heterologous transcriptional control elements.

Another way of increasing expression of desired genes can be the integration of multiple copies of genes into the genome. This can be accomplished in several ways. For example, the same cloned gene may be inserted into more than one locus (typically on different chromosomes) in the genome. Alternatively, different variations of the cloned gene, for example different promoter/terminator combinations, may be introduced into more than one locus. A combination of gene expression on a plasmid in addition to chromosomal expression could be used. Random integration techniques can also be used in which the location and copy number of an integrated gene are not known. A less frequently used approach could be to introduce tandem repeats of the gene and expression machinery into a single locus.

Where desired, the expression of one or more of the enzymes or fragments thereof provided herein is under the control of a regulatory sequence that controls directly or indirectly the expression in a time-dependent fashion during the fermentation. Inducible promoters can be used to achieve this.

In some cases, a cell/microorganism is transformed or transfected with a genetic vehicle, such as an expression vector comprising a heterologous polynucleotide sequence coding for an enzyme or fragment thereof. In some cases, the vector(s) can be an episomal vector, or the gene sequence can be integrated into the genome of the microorganism, or any combination thereof. In some cases, the vectors comprising the heterologous polynucleotide sequence encoding the enzymes or fragments thereof provided herein are integrated into the genome of the microorganism.

To facilitate insertion and expression of different genes coding for the enzymes of interest or fragments thereof, the constructs or expression vectors can be designed with at least one cloning site for insertion of any gene coding for such enzyme or fragment. The cloning site can be a multiple cloning site, e.g., containing multiple restriction sites.

Transfection and Transformation

Standard transfection techniques can be used to insert genes into a microorganism. As used herein, the term “transfection” or “transformation” can refer to the insertion of an exogenous nucleic acid or polynucleotide into a host cell. The exogenous nucleic acid or polynucleotide can be maintained as a non-integrated vector, for example, a plasmid or episomal vector, or alternatively, can be integrated into the host cell genome. The term transfecting or transfection is intended to encompass all conventional techniques for introducing nucleic acid or polynucleotide into cells/microorganisms. Examples of transfection techniques include, but are not limited to, lithium acetate mediated transformation, calcium phosphate precipitation, DEAE-dextran-mediated transfection, lipofection, electroporation, microinjection, rubidium chloride or polycation mediated transfection, protoplast fusion, and sonication. The transfection method that provides optimal transfection frequency and expression of the construct in the particular host cell line and type is favored. For stable transfectants, the constructs are integrated so as to be stably maintained within the host chromosome. In some cases, the preferred transfection is a stable transfection. In some cases, the integration of the gene occurs at a specific locus within the genome of the microorganism.

Expression vectors or other nucleic acids can be introduced to selected cells/microorganisms by any of a number of suitable methods. For example, vector constructs can be introduced to appropriate cells by any of a number of transformation methods. Standard calcium chloride-mediated bacterial transformation is still commonly used to introduce naked DNA to bacteria (see, e.g., Sambrook et al., 1989, Molecular Cloning, A Laboratory Manual, Cold Spring Harbor Laboratory Press, Cold Spring Harbor, N.Y.), but electroporation and conjugation can also be used (see, e.g., Ausubel et al., 1988, Current Protocols in Molecular Biology, John Wiley & Sons, Inc., NY, N.Y.).

For the introduction of vector constructs to yeast or other fungal cells, chemical transformation and electroporation methods can be used (e.g., Rose et al., 1990, Methods in Yeast Genetics, Cold Spring Harbor Laboratory Press, Cold Spring Harbor, N.Y.). Transformed cells can be isolated on selective media appropriate to the selectable marker used. Alternatively, or in addition, plates or filters lifted from plates can be scanned for GFP fluorescence to identify transformed clones.

For the introduction of vectors comprising differentially expressed sequences to certain types of cells, the method used can depend on the form of the vector. Plasmid vectors can be introduced by any of a number of transfection methods, including, for example, lipid-mediated transfection (“lipofection”), DEAE-dextran-mediated transfection, electroporation or calcium phosphate precipitation (see, e.g., Ausubel et al., 1988, Current Protocols in Molecular Biology, John Wiley & Sons, Inc., New York, N.Y.).

Lipofection reagents and methods suitable for transient transfection of a wide variety of transformed and non-transformed or primary cells are widely available, making lipofection an attractive method of introducing constructs to eukaryotic, and particularly mammalian cells in culture. Many companies offer kits and ways for this type of transfection.

The host cell can be capable of expressing the construct encoding the desired protein, processing the protein and transporting a secreted protein to the cell surface for secretion. Processing includes co- and post-translational modification such as leader peptide cleavage, GPI attachment, glycosylation, ubiquitination, and disulfide bond formation.

Cells/microorganisms can be transformed or transfected with the above-described expression vectors or polynucleotides coding for one or more enzymes as disclosed herein and cultured in nutrient media modified as appropriate for the specific cell/microorganism, inducing promoters, selecting transformants, or amplifying the genes encoding the desired sequences. In some cases, electroporation methods can be used to deliver an expression vector.

Expression of a vector (and the gene contained in the vector) can be verified by an expression assay, for example, qPCR, colony PCR, sequencing of a locus or whole genome sequencing, or by measuring levels of RNA. Expression level can be indicative also of copy number. For example, if expression levels are extremely high, this can indicate that more than one copy of a gene was integrated in a genome. Alternatively, high expression can indicate that a gene was integrated in a highly transcribed area, for example, near a highly expressed promoter. Expression can also be verified by measuring protein levels, such as through Western blotting.

CRISPR/Cas System

The methods disclosed throughout can involve pinpoint insertion of genes or the deletion of genes (or parts of genes). Methods described herein can use a CRISPR/Cas system. For example, double-strand breaks (DSBs) can be generated using a CRISPR/Cas system, e.g., a type II CRISPR/Cas system. A Cas enzyme used in the methods disclosed herein can be Cas9, which catalyzes DNA cleavage. Enzymatic action by Cas9 from Streptococcus pyogenes or any closely related Cas9 can generate double stranded breaks at target site sequences which hybridize to 20 nucleotides of a guide sequence and have a protospacer-adjacent motif (PAM) following the 20 nucleotides of the target sequence.

A vector can be operably linked to an enzyme-coding sequence encoding a CRISPR enzyme, such as a Cas protein and Mad7. Cas proteins that can be used include class 1 and class 2. Non-limiting examples of Cas proteins include Cas1, Cas1B Cas1, Cas1B, Cas2, Cas3, Cas4, Cas5, Cas5d, Cas5t, Cas5h, Cas5a, Cas6, Cas7, Cas8, Cas9 (also known as Csn1 or Csx12), Cas10, Csyl , Csy2, Csy3, Csy4, Cse1, Cse2, Cse3, Cse4, Cse5e, Csc1, Csc2, Csa5, Csn1, Csn2, Csm1, Csm2, Csm3, Csm4,

Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, C2c1, C2c2, C2c3, Cpf1, CARF, DinG, homologues thereof, or modified versions thereof. An unmodified CRISPR enzyme can have DNA cleavage activity, such as Cas9. A CRISPR enzyme can direct cleavage of one or both strands at a target sequence, such as within a target sequence and/or within a complement of a target sequence. For example, a CRISPR enzyme can direct cleavage of one or both strands within 1, 2, 3, 4, 5, 6, 7, 8, 9, 10, 15, 20, 25, 30, 35, 40, 45, 50, 60, 70, 80, 90, 100, 125, 150, 175, 200, 300, 400, 500, or more base pairs from the first or last nucleotide of a target sequence. A vector that encodes a CRISPR enzyme that is mutated to with respect, to a corresponding wild-type enzyme such that the mutated CRISPR enzyme lacks the ability to cleave one or both strands of a target polynucleotide containing a target sequence can be used.

A vector that encodes a CRISPR enzyme comprising one or more nuclear localization sequences (NLSs) can be used. For example, there can be 1, 2, 3, 4, 5, 6, 7, 8, 9, 10 NLSs used. A CRISPR enzyme can comprise the NLSs at or near the ammo-terminus (e.g., 1, 2, 3, 4, 5, 6, 7, 8, 9, 10 NLSs), or at or near the carboxy-terminus (e.g., 1, 2, 3, 4, 5, 6, 7, 8, 9, 10 NLSs), or any combination of these (e.g., one or more NLS at the ammo-terminus and one or more NLS at the carboxy terminus). When more than one NLS is present, each can be selected independently of others, such that a single NLS can be present in more than one copy and/or in combination with one or more other NLSs present in one or more copies.

CRISPR enzymes used in the methods can comprise at most 6 NLSs. An NLS is considered near the N- or C-terminus when the nearest amino acid to the NLS is within 50 amino acids along a polypeptide chain from the N- or C-terminus, e.g., within 1, 2, 3, 4, 5, 10, 15, 20, 25, 30, 40, or 50 amino acids.

Guide RNA

As used herein, the term “guide RNA” and its grammatical equivalents refers to an RNA that can specifically target a DNA sequence and form a complex with Cas protein. An RNA/Cas complex can assist in “guiding” Cas protein to a target DNA.

A method disclosed herein also can comprise introducing into a cell or embryo at least one guide RNA or nucleic acid, e.g., DNA encoding at least one guide RNA. A guide RNA can interact with a RNA-guided endonuclease to direct the endonuclease to a specific target site, at which site the 5′ end of the guide RNA base pairs with a specific protospacer sequence in a chromosomal sequence.

A guide RNA can comprise two RNAs, e.g., CRISPR RNA (crRNA) and transactivating crRNA (tracrRNA). A guide RNA can sometimes comprise a single-chain RNA, or single guide RNA (sgRNA) formed by fusion of a portion (e.g., a functional portion) of crRNA and tracrRNA. A guide RNA can also be a dualRNA comprising a crRNA and a tracrRNA. Furthermore, a crRNA can hybridize with a target DNA.

As discussed above, a guide RNA can be an expression product. For example, a DNA that encodes a guide RNA can be a vector comprising a sequence coding for the guide RNA. A guide RNA can be transferred into a cell or microorganism by transfecting the cell or microorganism with an isolated guide RNA or plasmid DNA comprising a sequence coding for the guide RNA and a promoter. A guide RNA can also be transferred into a cell or microorganism in other ways, such as using virus-mediated gene delivery.

A guide RNA can be isolated. For example, a guide RNA can be transfected in the form of an isolated RNA into a cell or microorganism. A guide RNA can be prepared by in vitro transcription using any in vitro transcription system. A guide RNA can be transferred to a cell in the form of isolated RNA rather than in the form of plasmid comprising encoding sequence for a guide RNA.

A guide RNA can comprise three regions: a first region at the 5′ end that can be complementary to a target site in a chromosomal sequence, a second internal region that can form a stem loop structure, and a third 3′ region that can be single-stranded. A first region of each guide RNA can also be different such that each guide RNA guides a fusion protein to a specific target site. Further, second and third regions of each guide RNA can be identical in all guide RNAs.

A first region of a guide RNA can be complementary to sequence at a target site in a chromosomal sequence such that the first region of the guide RNA can base pair with the target site. In some cases, a first region of a guide RNA can comprise from 10 nucleotides to 25 nucleotides (i.e., from 10 nucleotides to 25 nucleotides; or 10 nucleotides to 25 nucleotides; or from 10 nucleotides to 25 nucleotides; or from 10 nucleotides to 25 nucleotides or more. For example, a region of base pairing between a first region of a guide RNA and a target site in a chromosomal sequence can be 10, 11, 12, 13, 14, 15, 16, 17, 18, 19, 20, 22, 23, 24, 25, or more nucleotides in length. Sometimes, a first region of a guide RNA can be 19, 20, or 21 nucleotides in length.

A guide RNA can also comprise a second region that forms a secondary structure. For example, a secondary structure formed by a guide RNA can comprise a stem (or hairpin) and a loop. A length of a loop and a stem can vary. For example, a loop can range from 3 to 10 nucleotides in length, and a stem can range from 6 to 20 base pairs in length. A stem can comprise one or more bulges of 1 to 10 nucleotides. The overall length of a second region can range from 16 to 60 nucleotides in length. For example, a loop can be 4 nucleotides in length and a stem can be 12 base pairs.

A guide RNA can also comprise a third region at the 3′ end that can be essentially single-stranded. For example, a third region is sometimes not complementary to any chromosomal sequence in a cell of interest and is sometimes not complementary to the rest of a guide RNA. Further, the length of a third region can vary. A third region can be more than 4 nucleotides in length. For example, the length of a third region can range from 5 to 60 nucleotides in length.

A guide RNA can be introduced into a cell or embryo as an RNA molecule. For example, a RNA molecule can be transcribed in vitro and/or can be chemically synthesized. An RNA can be transcribed from a synthetic DNA molecule, e.g., a gBlocks® gene fragment. A guide RNA can then be introduced into a cell or embryo as an RNA molecule. A guide RNA can also be introduced into a cell or embryo in the form of a non-RNA nucleic acid molecule, e.g., DNA molecule. For example, a DNA encoding a guide RNA can be operably linked to promoter control sequence for expression of the guide RNA in a cell or embryo of interest. A RNA coding sequence can be operably linked to a promoter sequence that is recognized by RNA polymerase III (Pol III). Plasmid vectors that can be used to express guide RNA include, but are not limited to, px330 vectors and px333 vectors. In some cases, a plasmid vector (e.g., px333 vector) can comprise two guide RNA-encoding DNA sequences.

A DNA sequence encoding a guide RNA can also be part of a vector. Further, a vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. A DNA molecule encoding a guide RNA can also be linear. A DNA molecule encoding a guide RNA can also be circular.

When DNA sequences encoding an RNA-guided endonuclease and a guide RNA are introduced into a cell, each DNA sequence can be part of a separate molecule (e.g., one vector containing an RNA-guided endonuclease coding sequence and a second vector containing a guide RNA coding sequence) or both can be part of a same molecule (e.g., one vector containing coding (and regulatory) sequence for both an RNA-guided endonuclease and a guide RNA).

Site-Specific Insertion

Insertion of the genes can be site-specific. For example, one or more genes can be inserted adjacent to a promoter. Genes can also be inserted into a neutral location in a genome such as into a non-coding region or elsewhere such that wild-type gene function remains intact.

Modification of a targeted locus of a cell/microorganism can be produced by introducing DNA into cell/microorganisms, where the DNA has homology to the target locus. DNA can include a marker gene, allowing for selection of cells comprising the integrated construct. Homologous DNA in a target vector can recombine with DNA at a target locus. A marker gene can be flanked on both sides by homologous DNA sequences, a 3′ recombination arm, and a 5′ recombination arm.

A variety of enzymes can catalyze insertion of foreign DNA into a microorganism genome. For example, site-specific recombinases can be clustered into two protein families with distinct biochemical properties, namely tyrosine recombinases (in which DNA is covalently attached to a tyrosine residue) and serine recombinases (where covalent attachment occurs at a serine residue). In some cases, recombinases can comprise Cre, ΦC31 integrase (a serine recombinase derived from Streptomyces phage (13C31), or bacteriophage derived site-specific recombinases (including Flp, lambda integrase, bacteriophage HK022 recombinase, bacteriophage R4 integrase and phage TP901-1 integrase).

The CRISPR/Cas system can be used to perform site specific insertion. For example, a nick on an insertion site in the genome can be made by CRISPR/Cas to facilitate the insertion of a transgene at the insertion site.

The methods described herein, can utilize techniques that can be used to allow a DNA or RNA construct entry into a host cell include, but are not limited to, calcium phosphate/DNA coprecipitation, microinjection of DNA into a nucleus, electroporation, bacterial protoplast fusion with intact cells, transfection, lipofection, infection, particle bombardment, sperm mediated gene transfer, or any other technique.

Certain aspects disclosed herein can utilize vectors (including the ones described above). Any plasmids and vectors can be used as long as they are replicable and viable in a selected host microorganism. Vectors known in the art and those commercially available (and variants or derivatives thereof) can be engineered to include one or more recombination sites for use in the methods. Vectors that can be used include, but not limited to eukaryotic expression vectors such as pRS, pBluSkII, pET, pFastBac, pFastBacHT, pFastBacDUAL, pSFV, and pTet-Splice (Invitrogen), pEUK-C1, pPUR, pMAM, pMAMneo, pBI101, pBI121, pDR2, pCMVEBNA, and pYACneo (Clontech), pSVK3, pSVL, pMSG, pCH110, and pKK232-8 (Pharmacia, Inc.), pXT1, pSG5, pPbac, pMbac, pMClneo, and pOG44 (Stratagene, Inc.), and pYES2, pAC360, pBlueBa-cHis A, B, and C, pVL1392, pBlueBac111, pCDM8, pcDNA1, pZeoSV, pcDNA3, pREP4, pCEP4, and pEBVHis (Invitrogen, Corp.), and variants or derivatives thereof.

These vectors can be used to express a gene or portion of a gene of interest. A gene or portion of a gene can be inserted by using known methods, such as restriction enzyme or PCR-based techniques.

Fermentation

In some embodiments, the cells/microorganisms useful in the present invention should be cultured in fermentation conditions that are appropriate to convert a substrate to UDCA, cholic acid, and/or another UDCA precursor. Reaction conditions that should be considered include temperature, media flow rate, pH, media redox potential, agitation rate, inoculum level, maximum substrate concentrations, rates of introduction of the substrate to the bioreactor to ensure that substrate level does not become limiting, maximum product concentrations to avoid product inhibition, gas flow, gas composition, aeration rate, bio-reactor design, and media composition.

The optimum reaction conditions will depend partly on the particular cell/microorganism used. However, in some cases, it is preferred that the fermentation be performed at a pressure higher than ambient pressure.

The use of pressurized systems can greatly reduce the volume of the bioreactor required, and consequently the capital cost of the fermentation equipment. In some cases, reactor volume can be reduced in linear proportion to increases in reactor operating pressure, i.e. bioreactors operated at 10 atmospheres of pressure need only be one tenth the volume of those operated at 1 atmosphere of pressure.

Fermentation Conditions

In those embodiments in which the cell/microorganism is cultured in fermentation conditions, the pH of the culture media may be optimized based on the cell/microorganism used. For example, the pH used can range from 4 to 10. In other instances, the pH can be from 5 to 9; 6 to 8; 6.1 to 7.9; 6.2 to 7.8; 6.3 to 7.7; 6.4 to 7.6; 6.5 to 7.5; 6.6 to 7.4; or 5.5 to 7.5. For example, the pH can be from 6.6 to 7.4. In some cases, the pH can be from 5 to 9. In some cases, the pH can be from 6 to 8. In some cases, the pH can be from 6.1 to 7.9. In some cases, the pH can be from 6.2 to 7.8. In some cases, the pH can be from 6.3 to 7.7. In some cases, the pH can be from 6.4 to 7.6. In some cases, the pH can be from 6.5 to 7.5. In some instances the pH used for the fermentation can be greater than about 6. In some instances the pH used for the fermentation can be lower than about 10.

Temperature can also be adjusted based on the cell/microorganism used. For example, the temperature can range from 27° C. to 45° C.; 28° C. to 44° C.; 29° C. to 43° C.; 30° C. to 42° C.; 31° C. to 41° C.; 32° C. to 40° C.; or 36° C. to 39° C.

Availability of oxygen and other gases may affect yield and fermentation rate. For example, when considering oxygen availability, the percent of dissolved oxygen (DO) within the fermentation media can be from 1% to 40%. In certain instances, the DO concentration can be from 1.5% to 35%; 2% to 30%; 2.5% to 25%; 3% to 20%; 4% to 19%; 5% to 18%; 6% to 17%; 7% to 16%;

8% to 15%; 9% to 14%; 10% to 13%; or 11% to 12%. For example, in some cases the DO concentration can be from 2% to 30%. In other cases, the DO can be from 3% to 20%. In some cases, the DO can be from 4% to 10%. In some cases, the DO can be from 1.5% to 35%. In some cases, the DO can be from 2.5% to 25%. In some cases, the DO can be from 4% to 19%. In some cases, the DO can be from 5% to 18%. In some cases, the DO can be from 6% to 17%. In some cases, the DO can be from 7% to 16%. In some cases, the DO can be from 8% to 15%. In some cases, the DO can be from 9% to 14%. In some cases, the DO can be from 10% to 13%. In some cases, the DO can be from 11% to 12%.

In some cases, atmospheric CO2 can help to control the pH within cell culture medium. pH contained within cell culture media is dependent on a balance of dissolved CO₂ and bicarbonate (HCO₃). Changes in atmospheric CO₂ can alter the pH of the medium. In certain instances, the atmospheric CO₂ can be from 0% to 10%; 0.01% to 9%; 0.05% to 8%; 0.1% to 7%; 0.5% to 6%; 1% to 5%; 2% to 4%; 3% to 6%; 4% to 7%; 2% to 6%; or 5% to 10%.

In cases where a switch is used, the media can comprise the molecule that induces or represses the switch.

When a lanthanum switch is used to repress the expression of one or more of the genes described herein, the media can comprise lanthanum, which will repress expression of the one or more genes under the control of the switch. In the case of lanthanum any one of the following concentrations can effectively repress expression of the one or more genes: 0.1 μM; 0.5 μM; 1 μM; 2 μM; 3 μM; 4 μM; 5 μM; 6 μM; 7 μM; 8 μM; 9 μM; 10 μM; 12.5 μM; 15 μM; 17.5 μM; 20 μM; 25 μM; 50 μM; 100 μM or more. In one case, 0.1 μM lanthanum can be used to repression expression of the one or more genes under the control of a lanthanum switch. In other cases, at least 0.5 μM lanthanum can be used. In other cases, at least 1 μM lanthanum can be used. In other cases, at least 2 μM lanthanum can be used. In other cases, at least 3 μM lanthanum can be used. In other cases, at least 4 μM lanthanum can be used. In other cases, at least 5 μM lanthanum can be used. In other cases, at least 6 μM lanthanum can be used. In other cases, at least 7 μM lanthanum can be used. In other cases, at least 8 μM lanthanum can be used. In other cases, at least 9 μM lanthanum can be used. In other cases, at least 10 μM lanthanum can be used. In other cases, at least 12.5 μM lanthanum can be used. In other cases, at least 15 μM lanthanum can be used. In other cases, at least 17.5 μM lanthanum can be used. In other cases, at least 20 μM lanthanum can be used. In other cases, at least 25 μM lanthanum can be used. In other cases, at least 50 μM lanthanum can be used. In other cases, at least 100 μM lanthanum can be used. In some cases, a range of 0.5 μM lanthanum to 100 μM lanthanum will effectively repress gene expression. In some cases, a range of 0.5 μM lanthanum to 50 μM lanthanum will repress gene expression. In other cases, a range of 1 μM lanthanum to 20 μM lanthanum will repress gene expression. In some cases, a range of 2 μM lanthanum to 15 μM lanthanum will repress gene expression. In some cases, a range of 3 μμM lanthanum to 12.5 μM lanthanum will repress gene expression. In some cases, a range of 4 μμM lanthanum to 12 μM lanthanum will repress gene expression. In some cases, a range of 5 μM lanthanum to 11.5 μM lanthanum will repress gene expression. In some cases, a range of 6 μμM lanthanum to 11 μM lanthanum will repress gene expression. In some cases, a range of 7 μM lanthanum to 10.5 μM lanthanum will repress gene expression. In some cases, a range of 8 μμM lanthanum to 10 μM lanthanum will repress gene expression.

In some cases, the lanthanum in the media can be diluted to turn on expression of the one or more lanthanum repressed genes. For example, in some cases, the dilution of lanthanum containing media can be 1:1 (1 part lanthanum containing media to 1 part non-lanthanum containing media). In some cases, the dilution can be at least 1:2; 1:3; 1:4; 1:5; 1:7.5; 1:10; 1:15; 1:20; 1:25; 1:30; 1:35; 1:40; 1:45; 1:50; 1:75; 1:100; 1:200; 1:300; 1:400; 1:500; 1:1,000; or 1:10,000. For example, in some cases, a 1:2 dilution can be used. In some cases, at least a 1:3 dilution can be used. In some cases, at least a 1:4 dilution can be used. In some cases, at least a 1:5 dilution can be used. In some cases, at least a 1:7.5 dilution can be used. In some cases, at least a 1:10 dilution can be used. In some cases, at least a 1:15 dilution can be used. In some cases, at least a 1:20 dilution can be used. In some cases, at least a 1:25 dilution can be used. In some cases, at least a 1:30 dilution can be used. In some cases, at least a 1:35 dilution can be used. In some cases, at least a 1:40 dilution can be used. In some cases, at least a 1:45 dilution can be used. In some cases, at least a 1:50 dilution can be used. In some cases, at least a 1:75 dilution can be used. In some cases, at least a 1:100 dilution can be used. In some cases, at least a 1:200 dilution can be used. In some cases, at least a 1:300 dilution can be used. In some cases, at least a 1:400 dilution can be used. In some cases, at least a 1:500 dilution can be used. In some cases, at least a 1:1,000 dilution can be used. In some cases, at least a 1:10,000 dilution can be used.

In some cases, the cell/microorganism may be grown in media comprising lanthanum. The media can then be diluted to effectively turn on the expression of the lanthanum repressed genes. The cell/microorganism can be then grown in conditions to promote the production of desired products, such as UDCA, cholic acid, and/or other UDCA precursors (as disclosed throughout).

When a glucose to galactose switch is used to repress the expression of one or more of the genes described herein (e.g., when a GAL1 or GAL10 promoter is used), the media can comprise glucose, which will repress expression of the one or more genes under the control of the switch. In the case of glucose any one of the following concentrations can effectively repress expression of the one or more genes: 0.1%; 0.5%; 1%; 2%; 3%; 4%; 5%; 6%; 7%; 8%; 9%; 10%; 12.5%; 15%; 17.5%; 20%; 25%; 50%; 100% or more. In one case, 0.1% glucose can be used to repression expression of the one or more genes under the control of a glucose to galactose switch. In other cases, at least 0.5% glucose can be used. In other cases, at least 1% glucose can be used. In other cases, at least 2% glucose can be used. In other cases, at least 3% glucose can be used. In other cases, at least 4% glucose can be used. In other cases, at least 5% glucose can be used. In other cases, at least 6% glucose can be used. In other cases, at least 7% glucose can be used. In other cases, at least 8% glucose can be used. In other cases, at least 9% glucose can be used. In other cases, at least 10% glucose can be used. In other cases, at least 12.5% glucose can be used. In other cases, at least 15% glucose can be used. In other cases, at least 17.5% glucose can be used. In other cases, at least 20% glucose can be used. In other cases, at least 25% glucose can be used. In other cases, at least 50% glucose can be used. In other cases, at least 100% glucose can be used. In some cases, a range of 0.5% glucose to 100% glucose will effectively repress gene expression. In some cases, a range of 0.5% glucose to 50% glucose will repress gene expression. In other cases, a range of 1% glucose to 20% glucose will repress gene expression. In some cases, a range of 2% glucose to 15% glucose will repress gene expression. In some cases, a range of 3% glucose to 12.5% glucose will repress gene expression. In some cases, a range of 4% glucose to 12% glucose will repress gene expression. In some cases, a range of 5% glucose to 11.5% glucose will repress gene expression. In some cases, a range of 6% glucose to 11% glucose will repress gene expression. In some cases, a range of 7% glucose to 10.5% glucose will repress gene expression. In some cases, a range of 8% glucose to 10% glucose will repress gene expression.

In some cases, the glucose in the media can be diluted to turn on expression of the one or more glucose repressed genes. For example, in some cases, the dilution of glucose containing media can be 1:1 (1 part glucose containing media to 1 part non-glucose containing media). In some cases, the dilution can be at least 1:2; 1:3; 1:4; 1:5; 1:7.5; 1:10; 1:15; 1:20; 1:25; 1:30; 1:35; 1:40; 1:45; 1:50; 1:75; 1:100; 1:200; 1:300; 1:400; 1:500; 1:1,000; or 1:10,000. For example, in some cases, a 1:2 dilution can be used. In some cases, at least a 1:3 dilution can be used. In some cases, at least a 1:4 dilution can be used. In some cases, at least a 1:5 dilution can be used. In some cases, at least a 1:7.5 dilution can be used. In some cases, at least a 1:10 dilution can be used. In some cases, at least a 1:15 dilution can be used. In some cases, at least a 1:20 dilution can be used. In some cases, at least a 1:25 dilution can be used. In some cases, at least a 1:30 dilution can be used. In some cases, at least a 1:35 dilution can be used. In some cases, at least a 1:40 dilution can be used. In some cases, at least a 1:45 dilution can be used. In some cases, at least a 1:50 dilution can be used. In some cases, at least a 1:75 dilution can be used. In some cases, at least a 1:100 dilution can be used. In some cases, at least a 1:200 dilution can be used. In some cases, at least a 1:300 dilution can be used. In some cases, at least a 1:400 dilution can be used. In some cases, at least a 1:500 dilution can be used. In some cases, at least a 1:1,000 dilution can be used. In some cases, at least a 1:10,000 dilution can be used.

In cases where a switch is used, the media can comprise the molecule that de-represses the switch. For example, when a glucose to galactose switch is used to repress the expression of one or more of the genes described herein (e.g., when a GAL1 or GAL10 promoter is used), the media can comprise raffinose, which will de-repress expression of the one or more genes under the control of the switch. In the case of raffinose any one of the following concentrations can effectively repress expression of the one or more genes: 0.1%; 0.5%; 1%; 2%; 3%; 4%; 5%; 6%; 7%; 8%; %; 10%; 12.5%; 15%; 17.5%; 20%; 25%; 50%; 100% or more. In one case, 0.1% raffinose can be used to de-repress expression of the one or more genes under the control of a raffinose switch. In other cases, at least 0.5% raffinose can be used. In other cases, at least 1% raffinose can be used. In other cases, at least 2% raffinose can be used. In other cases, at least 3% raffinose can be used. In other cases, at least 4% raffinose can be used. In other cases, at least 5% raffinose can be used. In other cases, at least 6% raffinose can be used. In other cases, at least 7% raffinose can be used. In other cases, at least 8% raffinose can be used. In other cases, at least 9% raffinose can be used. In other cases, at least 10% raffinose can be used. In other cases, at least 12.5% raffinose can be used. In other cases, at least 15% raffinose can be used. In other cases, at least 17.5% raffinose can be used. In other cases, at least 20% raffinose can be used. In other cases, at least 25% raffinose can be used. In other cases, at least 50% raffinose can be used. In other cases, at least 100% raffinose can be used. In some cases, a range of 0.5% raffinose to 100% raffinose will effectively repress gene expression. In some cases, a range of 0.5% raffinose to 50% raffinose will de-repress gene expression. In other cases, a range of 1% raffinose to 20% raffinose will repress gene expression. In some cases, a range of 2% raffinose to 15% raffinose will repress gene expression. In some cases, a range of 3% raffinose to 12.5% raffinose will de-repress gene expression. In some cases, a range of 4% raffinose to 12% raffinose will de-repress gene expression. In some cases, a range of 5% raffinose to 11.5% raffinose will de-repress gene expression. In some cases, a range of 6% raffinose to 11% raffinose will de-repress gene expression. In some cases, a range of 7% raffinose to 10.5% raffinose will de-repress gene expression. In some cases, a range of 8% raffinose to 10% raffinose will de-repress gene expression.

In cases where a switch is used, the media can comprise the molecule that induces the switch. For example, when a glucose to galactose switch is used to induce the expression of one or more of the genes (e.g., when a GAL1 or GAL10 promoter is used), the media can comprise galactose, which will induce expression of the one or more genes under the control of the switch. In the case of galactose any one of the following concentrations can effectively induce expression of the one or more genes: 0.1%; 0.5%; 1%; 2%; 3%; 4%; 5%; 6%; 7%; 8%; 9%; 10%; 12.5%; 15%; 17.5%; 20%; 25%; 50%; 100% or more. In one case, 0.1% galactose can be used to induce expression of the one or more genes under the control of a glucose to galactose switch. In other cases, at least 0.5% galactose can be used. In other cases, at least 1% galactose can be used. In other cases, at least 2% galactose can be used. In other cases, at least 3% galactose can be used. In other cases, at least 4% galactose can be used. In other cases, at least 5% galactose can be used. In other cases, at least 6% galactose can be used. In other cases, at least 7% galactose can be used. In other cases, at least 8% galactose can be used. In other cases, at least 9% galactose can be used. In other cases, at least 10% galactose can be used. In other cases, at least 12.5% galactose can be used. In other cases, at least 15% galactose can be used. In other cases, at least 17.5% galactose can be used. In other cases, at least 20% galactose can be used. In other cases, at least 25% galactose can be used. In other cases, at least 50% galactose can be used. In other cases, at least 100% galactose can be used. In some cases, a range of 0.5% galactose to 100% galactose will effectively induce gene expression. In some cases, a range of 0.5% galactose to 50% galactose will induce gene expression. In other cases, a range of 1% galactose to 20% galactose will induce gene expression. In some cases, a range of 2% galactose to 15% galactose will induce gene expression. In some cases, a range of 3% galactose to 12.5% galactose will induce gene expression. In some cases, a range of 4% galactose to 12% galactose will induce gene expression. In some cases, a range of 5% galactose to 11.5% galactose will induce gene expression. In some cases, a range of 6% galactose to 11% galactose will induce gene expression. In some cases, a range of 7% galactose to 10.5% galactose will induce gene expression. In some cases, a range of 8% galactose to 10% galactose will induce gene expression.

When a copper switch is used to induce the expression of one or more of the genes described herein, the media can comprise copper, which will induce expression of the one or more genes under the control of the switch. In the case of copper any one of the following concentrations can effectively induce expression of the one or more genes: 1 μM; 2.5 μM; 5 μM; 10 μM; 25 μM; 50 μM; 75 μM; 100 μM; 150 μM; 200 μM; 300 μM; 400 μM; 500 μM; 600 μM; 700 μM; 800 μM; 900 μM; 1 M; 10 mM or more. In one case, 1 μM copper can be used to induce expression of the one or more genes under the control of a copper promoter. In other cases, at least 5 μμM copper can be used. In other cases, at least 10 04 copper can be used. In other cases, at least 25 μMcopper can be used. In other cases, at least 50 μM copper can be used. In other cases, at least 100 μM copper can be used. In other cases, at least 200 μM copper can be used. In other cases, at least 300 μM copper can be used. In other cases, at least 400 μM copper can be used. In other cases, at least 500 μM copper can be used. In other cases, at least 600 μM copper can be used. In other cases, at least 700 μM copper can be used. In other cases, at least 800 μM copper can be used. In other cases, at least 900 μM copper can be used. In other cases, at least 1 mM copper can be used. In other cases, at least 2.5 mM copper can be used. In other cases, at least 5 mM copper can be used. In other cases, at least 7.5 mM copper can be used. In other cases, at least 10 mM copper can be used. In some cases, a range of 1 μM copper to 10 mM copper will effectively repress gene expression. In some cases, a range of 2.5 μM copper to 1 mM copper will repress gene expression. In other cases, a range of 5 μM copper to 800 μM copper will repress gene expression. In some cases, a range of 10 μM copper to 600 μM copper will repress gene expression. In some cases, a range of 25 μM copper to 500 μM copper will repress gene expression. In some cases, a range of 50 μM copper to 450 μM copper will repress gene expression. In some cases, a range of 75 μM copper to 400 μM copper will repress gene expression. In some cases, a range of 100 μM copper to 350 μM copper will repress gene expression. In some cases, a range of 150 μμM copper to 300 μM copper will repress gene expression. In some cases, a range of 200 μM copper to 250 μM copper will repress gene expression.

Bioreactor

Fermentation reactions can be carried out in any suitable bioreactor. In some cases, the bioreactor can comprise a first, growth reactor in which the cells/microorganisms are cultured, and a second, fermentation reactor, to which broth from the growth reactor is fed and in which most of the fermentation product is produced.

Product Recovery

The fermentation of the cells/microorganisms disclosed herein can produce a broth comprising a desired product (e.g., UDCA, cholic acid, and/or other UDCA precursor), one or more by-products, and/or the cell/microorganism itself.

In certain methods of producing products, the concentration of products in the fermentation broth is at least 0.1 g/L. For example, the concentration of products produced in the fermentation broth can be from 0.1 g/L to 0.5 g/L, 0.5 g/L to 1 g/L, 1 g/L to 5 g/L, 2 g/L to 6 g/L, 3 g/L to 7 g/L, 4 g/L to 8 g/L, 5 g/L to 9 g/L, or 6 g/L to 10 g/L. In some cases, the concentration of products can be at least 9 g/L. In some cases, the concentration of products can be from 0.1 g/L to 10 g/L. In some cases, the concentration of products can be from 0.5 g/L to 3 g/L. In some cases, the concentration of products can be from 1 g/L to 5 g/L. In some cases, the concentration of products can be from 2 g/L to 6 g/L. In some cases, the concentration of products can be from 3 g/L to 7 g/L. In some cases, the concentration of products can be from 4 g/L to 8 g/L. In some cases, the concentration of products can be from 5 g/L to 9 g/L. In some cases, the concentration of products can be from 6 g/L to 10 g/L. In some cases, the concentration of products can be from 1 g/L to 3 g/L. In some cases, the concentration of products can be about 2 g/L.

As discussed above, in certain cases the product produced in the fermentation reaction is converted to a different organic product. For example, the product produced may be a UDCA precursor that serves as a substrate for the further production of UDCA, cholic acid, or another UDCA precursor. In other cases, the product is first recovered from the fermentation broth before conversion to a different organic product.

In some cases, the product can be continuously removed from a portion of broth and recovered as purified. In particular cases, the recovery of the product includes passing the removed portion of the broth containing the product through a separation unit to separate the cells/microorganisms from the broth, to produce a cell-free product permeate, and returning the microorganisms to the bioreactor. The cell-free product containing permeate can then can be stored or be used for subsequent conversion to a different desired product.

The recovering of the desired product and/or one or more other products or by-products produced in the fermentation reaction can comprise continuously removing a portion of the broth and recovering separately the product and one or more other products from the removed portion of the broth. In some cases, the recovery of the product and/or one or more other products includes passing the removed portion of the broth containing the product and/or one or more other products through a separation unit to separate cells/microorganisms from the product and/or one or more other products, to produce a cell-free product and one or more other product-containing permeate, and returning the microorganisms to the bioreactor.

In the above cases, the recovery of the product and one or more other products can include first removing the product from the cell-free permeate followed by removing the one or more other products from the cell-free permeate. The cell-free permeate can also then returned to the bioreactor.

The product, or a mixed product stream containing the product, can be recovered from the fermentation broth. For example, methods that can be used can include but are not limited to, fractional distillation or evaporation, pervaporation, and extractive fermentation. Further examples include: recovery using steam from whole fermentation broths; reverse osmosis combined with distillation; liquid-liquid extraction techniques involving solvent extraction of the product; aqueous two-phase extraction of the product in PEG/dextran system; solvent extraction using alcohols or esters, e.g., ethyl acetate, tributylphosphate, diethyl ether, n-butanol, dodecanol, oleyl alcohol, and an ethanol/phosphate system; aqueous two-phase systems composed of hydrophilic solvents and inorganic salts. See generally, Voloch, M., et al., (1985) and U.S. Pat. Pub. Appl. No. 2012/0045807.

In some cases, the product and/or other by-products may be recovered from the fermentation broth by continuously removing a portion of the broth from the bioreactor, separating microbial cells from the broth (conveniently by filtration, for example), and recovering the product and others such as alcohols and acids from the broth. Alcohols can conveniently be recovered for example by distillation, and acids can be recovered for example by adsorption on activated charcoal. The separated microbial cells are returned to the fermentation bioreactor. The cell-free permeate remaining after the alcohol(s) and acid(s) have been removed is also preferably returned to the fermentation bioreactor. Additional nutrients can be added to the cell-free permeate to replenish the nutrient medium before it is returned to the bioreactor.

Also, if the pH of the broth is adjusted during recovery of the product and/or by-products, the pH should be re-adjusted to a similar pH to that of the broth in the fermentation bioreactor, before being returned to the bioreactor.

In Vitro Methods and Steps

In some embodiments, the present invention relates in part to an in vitro method of making UDCA or UDCA precursor. In other words, in these embodiments, the method does not involve the use of a microorganism. For example, the substrate may be contacted with an enzyme or a fragment thereof, such as described previously, in a medium.

In some embodiments, the method involves both in vivo and in vitro steps. For example, some reactions along the biosynthetic pathway can occur within a cell, whereas some of the reactions along the pathway occur outside of a cell. In certain such methods, a UDCA precursor may be secreted by a cell into media and then directly converted enzymatically or non-enzymatically (e.g., chemically) into a different product, such as UDCA or another DCA precursor.

CoEnyme A

The microorganism and methods described throughout can be used to produce a CoA-form of the products described throughout. In some cases, a CoA ligase can be used to produce a CoA form of any of the products described throughout.

In some cases, SLC27A5 can produce a CoA product that is (25R)-3α,7α-dihydroxy-5β-cholestanoyl-CoA or (25R)-3α,7α,12α-trihydroxy-5β-cholestanoyl-CoA. In some cases, AMACR can produce a CoA product that is (25S)-3α,7α-dihydroxy-5β-cholestanoyl-CoA or (25S)-3α,7α,12α-trihydroxy-5β-cholestanoyl-CoA. In some cases, ACOX2 can produce a CoA product that is (24E)-3α,7α-dihydroxy-5β-cholest-24-enoyl-CoA or (24E)-3α,7α,12α-trihydroxy-5β-cholest-24-enoyl-CoA. In some cases, HSD17B4 can produce a CoA product that is 3α,7α-dihydroxy-24-oxo-5β-cholestanoyl-CoA or 3α,7α,12α-trihydroxy-24-oxo-5β-cholestanoyl-CoA. In some cases, SCP2/Thiolase can produce a CoA product that is 3α,7α-dihydroxy-5β-cholan-24-oyl-CoA (CDC-CoA) or 3α,7α,12α-trihydroxy-5β-cholan-24-oyl-CoA. In some cases, 7α-HSD can produce a CoA product that is 3α-hydroxy-7-oxo-5β-cholan-24-oyl-CoA. In some cases, 7β-HSD can produce a CoA product that is 3α,7β-dihydroxy-5β-cholan-24-oyl-CoA (UDC-CoA).

In some cases, the CoA form of one or more of the products can be (25R)-3α,7α-dihydroxy-5β-cholestanoyl-CoA; (25R)-3α,7α,12α-trihydroxy-5β-cholestanoyl-CoA; (25S)-3α,7α-dihydroxy-5β-cholestanoyl-CoA; (25S)-3α,7α,12α-trihydroxy-5β-cholestanoyl-CoA; (24E)-3α,7α-dihydroxy-5β-cholest-24-enoyl-CoA; (24E)-3α,7α,12α-trihydroxy-5β-cholest-24-enoyl-CoA; 3α,7α-dihydroxy-24-oxo-5β-cholestanoyl-CoA; 3α,7α,12α-trihydroxy-24-oxo-5β-cholestanoyl-CoA; 3α,7α-dihydroxy-5β-cholan-24-oyl-CoA (CDC-CoA); 3α,7α,12α-trihydroxy-5β-cholan-24-oyl-CoA; 3α-hydroxy-7-oxo-5β-cholan-24-oyl-CoA; 3α,7β-dihydroxy-5β-cholan-24-oyl-CoA (UDC-CoA); or any combination thereof.

The products as disclosed throughout can be isolated in their CoA form.

Free Acids

The microorganism and methods described throughout can be used to produce a free acid-form of the products described throughout. In some cases, a hydrolase can be used to produce a free acid form of any of the products described throughout.

In some cases, CYP27A1 can produce a free acid product that is (25R)-3α,7α-dihydroxy-5β-cholestanoic acid or (25R)-3α,7α,12α-trihydroxy-5β-cholestan-26-oic acid. In some cases, SLC27A5 can produce a free acid product that is (25R)-3α,7α-dihydroxy-5β-cholestanoic acid or (25R)-3α,7α,12α-trihydroxy-5β-cholestan-26-oic acid. In some cases, AMACR can produce a free acid product that is (25S)-3α,7α-dihydroxy-5β-cholestanoic acid or (25S)-3α,7α,12α-trihydroxy-5β-cholestanoic acid. In some cases, ACOX2 can produce a free acid product that is (24E)-3α,7α-dihydroxy-5β-cholest-24-enoic acid or (24E)-3α,7α,12α-trihydroxy-5β-cholest-24-enoic acid. In some cases, HSD17B4 can produce a free acid product that is 3α,7α-dihydroxy-24-oxo-5β-cholestanoic acid or 3α,7α,12α-trihydroxy-24-oxo-5β-cholestanoic acid. In some cases, SCP2/Thiolase can produce a free acid product that is 3α,7α-dihydroxy-5β-cholanoic acid (chenodeoxycholic acid; CDCA) or 3α,7α,12α-trihydroxy-5β-cholan-24-oic acid (cholic acid). In some cases, 7α-HSD can produce a free acid product that is 3α-hydroxy-7-oxo-5β-cholanoic acid (nutriacholic acid; NCA). In some cases, 7β-HSD can produce a free acid product that is 3α,7β-dihydroxy-5β-cholanoic acid (ursodeoxycholic acid; UDCA). In some cases, Choloyl-CoA hydrolase can produce a free acid product that is UDCA or 3α,7α,12α-trihydroxy-5β-cholan-24-oic acid (cholic acid).

In some cases, the free acid form of one or more of the products can be (25R)-3α,7α-dihydroxy-5β-cholestanoic acid; (25R)-3α,7α,12α-trihydroxy-5β-cholestan-26-oic acid; (25R)-3α,7α-dihydroxy-5β-cholestanoic acid; (25R)-3α,7α,12α-trihydroxy-5β-cholestan-26-oic acid; (25S)-3α,7α-dihydroxy-5β-cholestanoic acid; (25S)-3α,7α,12α-trihydroxy-5β-cholestanoic acid; (24E)-3α,7α-dihydroxy-5β-cholest-24-enoic acid; (24E)-3α,7α,12α-trihydroxy-5β-cholest-24-enoic acid; 3α,7α-dihydroxy-24-oxo-5β-cholestanoic acid; 3α,7α,12α-trihydroxy-24-oxo-5β-cholestanoic acid; 3α,7α-dihydroxy-5β-cholanoic acid (chenodeoxycholic acid; CDCA); 3α,7α,12α-trihydroxy-5β-cholan-24-oic acid (cholic acid); 3α-hydroxy-7-oxo-5β-cholanoic acid (nutriacholic acid; NCA); 3α,7β-dihydroxy-5β-cholanoic acid (ursodeoxycholic acid; UDCA); 3α,7α,12α-trihydroxy-5β-cholan-24-oic acid (cholic acid); or any combination thereof.

The products as disclosed throughout can be isolated in their free acid form.

Compositions

The present invention also relates in part to a composition comprising UDCA or UDCA precursor, a free acid or CoA thereof, or a pharmaceutically-acceptable derivative or prodrug thereof. The composition may further comprise an excipient. The composition may be in the form of a medicament. A “pharmaceutically acceptable derivative” means any pharmaceutically acceptable salt, ester, salt of an ester, pro-drug or other derivative thereof. Pharmaceutically acceptable salts of the compounds of this invention include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate and undecanoate. Salts derived from appropriate bases include alkali metal (e.g., sodium), alkaline earth metal (e.g., magnesium), ammonium and N-(alkyl)₄⁺ salts.

The present invention also relates in part to a method of formulating the UDCA or UDCA precursor into a pharmaceutical composition.

For preparing pharmaceutical compositions from the compounds of the present invention, pharmaceutically-acceptable carriers include either solid or liquid carriers. Solid form preparations include powders, tablets, pills, capsules, cachets, suppositories, and dispersible granules. A solid carrier can be one or more substances, which also acts as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton Pa.

In powders, the carrier is a finely divided solid, which is in a mixture with the finely divided active component. In tablets, the active component is mixed with the carrier having the necessary binding properties in suitable proportions and compacted in the shape and size desired.

Suitable solid excipients are carbohydrate or protein fillers include, but are not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents are added, such as the cross-linked polyvinyl pyrrolidone, agar, alginic acid, or a salt thereof, such as sodium alginate.

Liquid form preparations include solutions, suspensions, and emulsions, for example, water or water/propylene glycol solutions. For parenteral injection, liquid preparations can be formulated in solution in aqueous polyethylene glycol solution.

The pharmaceutical preparation can be a unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The present invention also relates to a method of making the pharmaceutical composition. In some cases, UDCA or a UDCA precursor is mixed with an excipient to produce a pharmaceutical composition.

Treatment of Disease and Symptoms of Disease

The UDCA or UDCA precursors (or other free acids or CoA products as disclosed throughout) can be used to treat disease. This includes treating one or more symptoms of the diseases. For example, the UDCA or a UDCA precursor (or other free acids or CoA products as disclosed throughout) can be used to treat one of more of the following diseases: gallstones (e.g., cholesterol gallstones), primary biliary cirrhosis, cystic fibrosis, impaired bile flow, intrahepatic cholestasis of pregnancy, and/or cholelithiasis.

Some of the diseases or symptom of disease can be exclusive to humans, but other diseases or symptom of disease can be shared in more than one animal, such as in all mammals.

The present invention relates in part to a method of treating a disease or symptom of a disease, the method comprising administering UDCA or UDCA precursor, a free acid or CoA thereof, or a pharmaceutically-acceptable derivative or prodrug thereof, to a subject in need of such treatment.

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

Use of UDCA or UDCA precursor

The present invention further relates in part to the use of the UDCA or UDCA precursor made using the aforementioned method in the manufacture of a medicament for the treatment or a disease or symptom of a disease. The disease or symptom of a disease may be any disease or symptom capable of being treated by UDCA or the UDCA precursor. Examples of such include gallstones, primary biliary cirrhosis, cystic fibrosis, impaired bile flow, intrahepatic cholestasis of pregnancy, and cholelithiasis.

UDCA can be used to treat gallstones and is a byproduct of intestinal bacteria.

The UDCA precursors may be used to make other products, such as other UDCA precursors or UDCA.

### EXAMPLES

While some cases have been shown and described herein, such cases are provided by way of example only. Numerous variations, changes, and substitutions will now occur to those skilled in the art without departing from the invention. It should be understood that various alternatives to the cases of the invention described herein will be employed in practicing the invention.

### Example 1—Identification of Enzymes that Convert Sugar to UDCA and Generating Strains that can Make UDCA

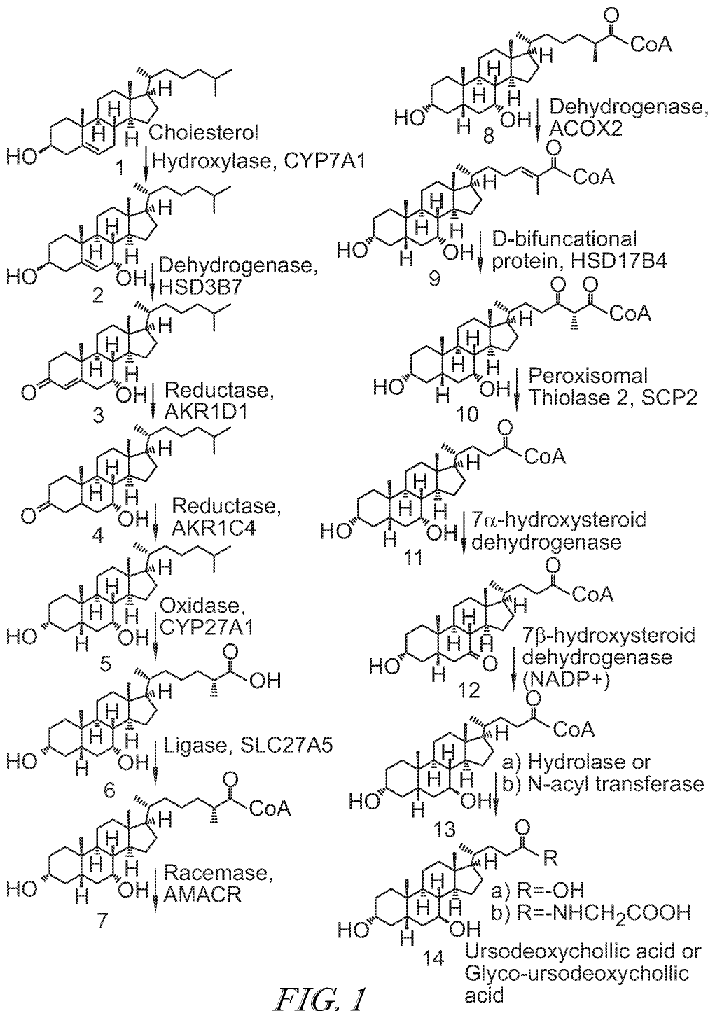

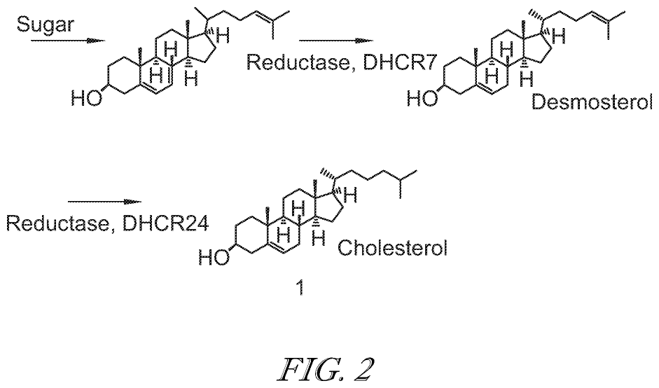

Thirteen heterologous enzymes (from the perspective of a Saccharomyces cerevisiae) were identified as possible enzymes that could be used to make UDCA from cholesterol. See e.g., FIG. 1. Two (2) additional enzymes were also identified as possible enzymes that could be used to convert sugar to cholesterol. See e.g., FIG. 2.

Genes encoding these enzymes were synthesized and then cloned into either yeast expression plasmids or into integration constructs. These plasmids or integrations constructs were subsequently transformed into Saccharomyces cerevisiae using standard yeast chemical transformation protocol, utilizing Lithium Acetate and PEG (3350). The transformed yeast were grown to mid log phase, then centrifuged at 4000 rpm with the supernatant removed. Pellets were washed with water and centrifuged again. The resulting pellet was resuspended in master mix containing 100 mM Lithium Acetate, 40% PEG (MW 3,350), 0.35 mg/ml carrier DNA (sheared salmon sperm DNA), and 50 to 500 ng of DNA to be transformed. The cell suspension was then incubated at 30° C. for 30 minutes, followed by at 45 minute heat shock at 42° C. At this point, nutritional selection was plated, while antifungal selection underwent a 4 hr to overnight recovery in rich yeast media before plating on agar containing the antifungal drug. Plates were then incubated at 30° C. for 2 to 3 days. After colonies were formed, proper integrations were verified by colony PCR before using strain in experiments.

Table 1 shows representative genes that were expressed in the yeast strains and the genetic origin of the enzymes that exhibited the best activity. Genes from other sources were also found to be active, but are not represented on Table 1.

TABLE 1

| Gene/enzyme | SEQ ID NO(s). | Source of Variants |
|---|---|---|
| ADR | 239 | Bovine |
| ADX | 241, 243, 245, 247, | Bovine, Zebrafish, |
| | 249, 251, 253, 255, | human |
| | 257, 259, 261 | |
| DHCR7 | 1 | Arabidopsis |
| DHCR24 | 21, 23, 25, 27, 45, 47 | Human, Bovine, |
| | | Zebrafish |
| CYP7A1 | 53, 65, 67, 69, 71, 73, | Mouse |
| | 75, 77, 79 | |
| HSD3B7 | 81 | Human |
| AKR1D1 | 91 | Mouse |
| AKR1C4 | 101 | Macaca fuscata |
| CYP27A1 | 125, 129, 131 | Rat, Mouse, Bovine |
| SLC27A5 | 139 | Human |
| AMACR | 145, 147 | Rat, Human |
| ACOX2 | 159, 165 | Human, Rabbit |
| HSD17B4 | 179, 183, 189 | Rat, Bovine, Xenopus |
| SCP2 | 203 | Yeast (POT1) |
| 7alpha- | 207, 211 | Escherichia coli, |
| hydroxysteroid | | Bacteroides |
| dehydrogenase | | fragilis |
| 7beta- | 221 | Clostridium |
| hydroxysteroid | | sardiniense |
| dehydrogenase | | |
| (NADP+) | | |

### Example 2—Yeast Strains having the Ability to Produce Cholesterol

Saccharomyces cerevisiae, which does not have the ability to naturally produce cholesterol, were genetically modified to upregulate the mevalonate pathway by overexpressing S. cerevisiae tHMG1 driven by a pGAL1 promoter. Additionally, S. cerevisiae were also genetically modified to express two heterologous genes, DHCR7 and DHCR24 driven by a GAL1 or GAL10 promoter.

All strains expressed the same DCHR7 from A. thaliana.

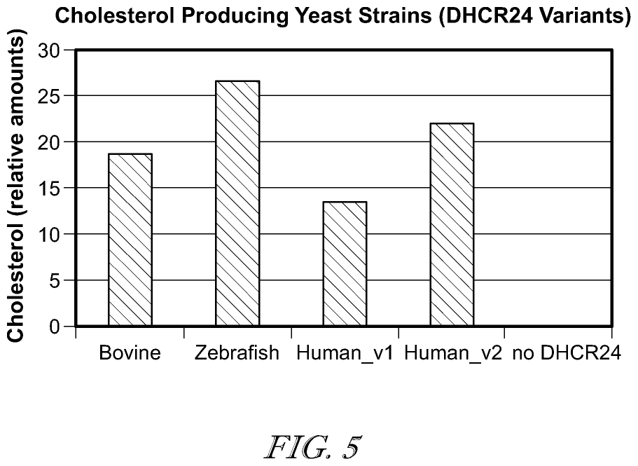

These different strains were tested for their ability to produce sterol compounds using GC/MS. As shown in FIG. 5, yeast strains expressing a DHCR24, were capable of making cholesterol, where DHCR24 from Homo sapiens and Danio rerio (zebrafish) had the best activity. The yeast strains that did not have a DHCR24 gene, did not produce any cholesterol.

### Example 3—Converting Cholesterol to 7-alpha-hydrogcholesterol

S. cerevisiae expressing A. thaliana DHCR7 and H. sapiens DHCR24 were transformed with several variants of cytochrome p450 family 7 subfamily A member 1 (CYP7A1) in combination with different adrenodoxin (ADX) variants. All strains expressed Bos taurus adrenodoxin reductases (ADRs).

The strains were then tested for their ability to convert cholesterol to 7-alpha-hydroxycholesterol, by its ability to hydroxylate the C7 carbon in cholesterol molecules. This conversion was detected by GC/MS.

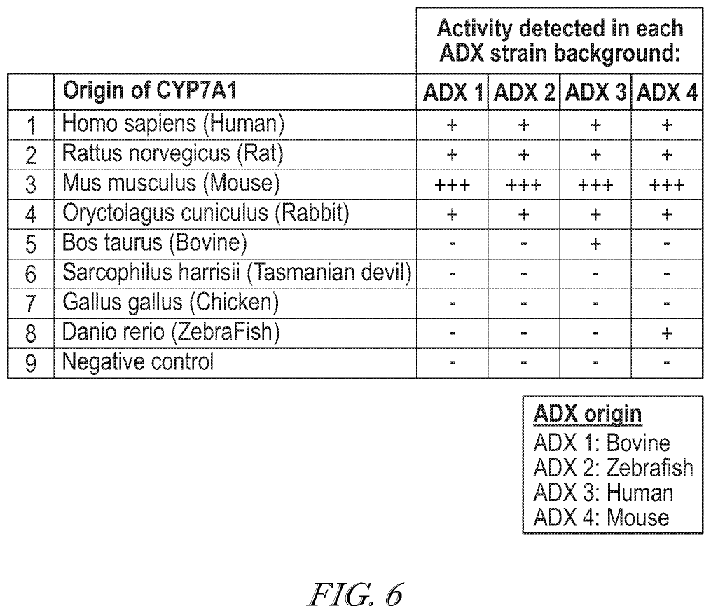

As shown in FIG. 6, CYP7A1 from Mus musculus exhibited the best activity. Activity was also seen in CYP7A1 from Homo sapiens, Rattus norvegicus, Ogctolagus cuniculus, Bos taurus, and Danio rerio.

### Example 4—Converting 7-alpha-hydroxycholesterol to 7α-hydroxy-4-cholesten-3-one

Strains expressing A. thaliana DHCR7 and H. sapiens DHCR24 were genetically engineered to further express M. musculus CYP7A1, ADX from B. taurus and D. rerio, B. taurus adrenodoxin reductase (ADR), and 3 beta-hydroxysteroid dehydrogenase type 7 (HSD3B7).

The strains were then tested by GC/MS for their ability to convert 7-alpha-hydroxycholesterol to 7α-hydroxy-4-cholesten-3-one.

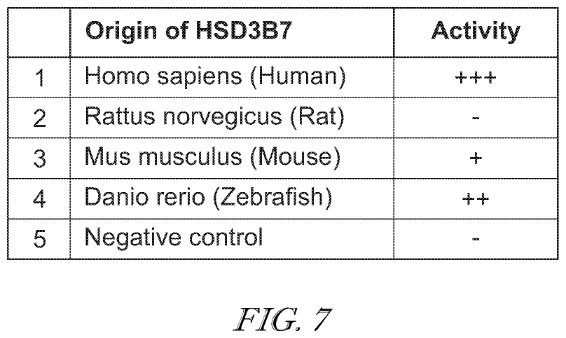

As shown in FIG. 7, HSD3B7 from Homo sapiens exhibited the best activity. Activity was also seen in HSD3B7 from Mus musculus and Danio rerio.

### Example 5—Converting 7α-hydroxy-4-cholesten-3-one to 7α-hydroxy-5β-cholestan-3-one

Strains expressing A. thaliana DHCR7 and H. sapiens DHCR24 were genetically engineered to further express M. musculus CYP7A1, ADX from D. rerio and B. taurus, B. taurus ADR, H. sapiens HSD3B7, and aldo-keto reductase family 1 member D1 (AKR1D1).

The strains were then tested by GC/MS for their ability to convert 7α-hydroxy-4-cholesten-3-one to 7α-hydroxy-5β-cholestan-3-one.

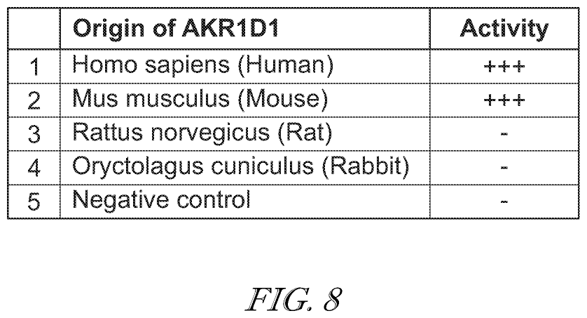

As shown in FIG. 8, AKR1D1 from Homo sapiens and Mus musculus exhibited the best activity.

### Example 6—Converting 7α-hydroxy-5β-cholestan-3-one to 5β-cholestane-3α,7α-diol

Strains expressing A. thaliana DHCR7 and H. sapiens DHCR24 were genetically engineered to further express M. musculus CYP7A1, ADX from D. rerio and B. taurus, B. taurus ADR, H. sapiens HSD3B7, M. musculus AKR1D1, and aldo-keto reductase family 1 member C9 (AKR1C9) or aldo-keto reductase family 1 member C4 (AKR1C4).

The strains were then tested by GC/MS for their ability to convert 7α-hydroxy-5β-cholestan-3-one to 5β-cholestane-3α,7α-diol.

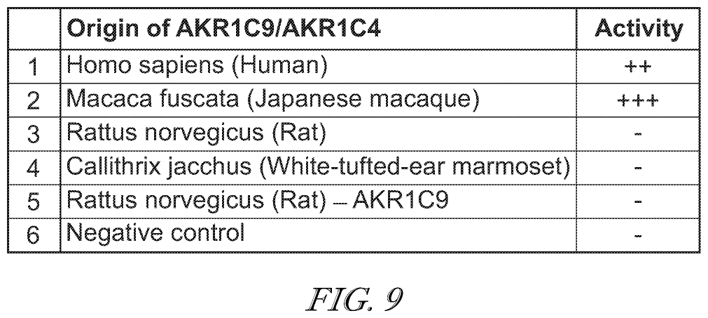

As shown in FIG. 9, AKR1C4 from Macaca fuscata exhibited the best activity. Additionally, AKR1C4 from Homo sapiens exhibited very good activity.

### Example 7—Converting 7α-hydroxy-4-cholesten-3-one to 7α,12α-dihydrog-4-cholesten-3-one

Strains expressing A. thaliana DHCR7 and H. sapiens DHCR24 were genetically engineered to further express M. musculus CYP7A1, ADX from D. rerio and B. taurus, B. taurus ADR, H. sapiens HSD3B7, and CYP8B1.

The strains were then tested by GC/MS for their ability to add a third hydroxyl group to the C12 in the cholesterol backbone. The strains were tested for their ability to produce 7α,12α-dihydroxy-4-cholesten-3-one from 7α-hydroxy-4-cholesten-3-one.

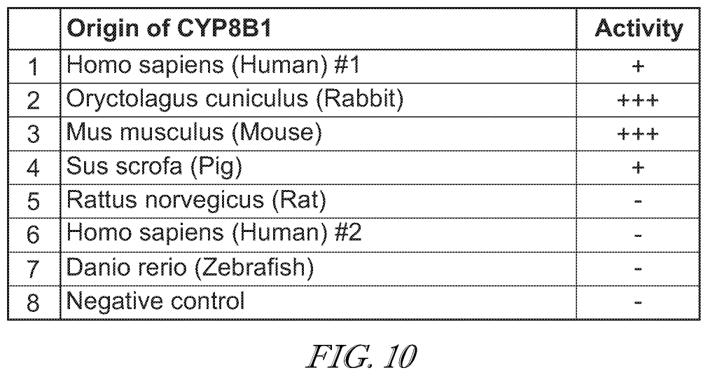

As shown in FIG. 10, CYP8B1 from Mus musculus and Ogctolagus cuniculus exhibited the best activity. CYP8B1 from Homo sapiens and Sus scrofa also exhibited activity.

### Example 8—Converting 5β-cholestane-3α,7α-diol to (25R)-3α,7α-dihydrog-5β-cholestanoic acid (and Further to (25R)-3α,7α-dihydroxy-5β-cholestanoyl-CoA by Coupling with SLC27A5)

Strains expressing A. thaliana DHCR7 and H. sapiens DHCR24 and also transformed with other enzymes necessary to produce 5β-cholestane-3α,7α-diol were further genetically engineered to further express different CYP27A1 variants. 7 variants of CYP27A1 were tested in combination with 2 variants of ADX (D. rerio and B. taurus) and B. taurus ADR. Additionally, H. sapiens SLC27A5 was expressed to couple this CYP27A1 activity, allowing for detection of the SLC27A5 product by LC-MS instead.

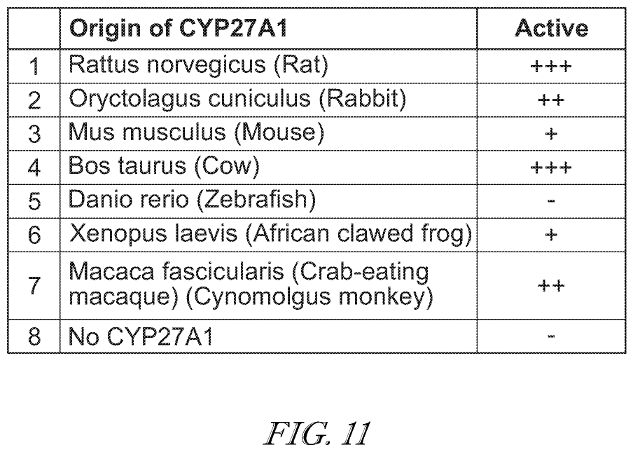

As shown in FIG. 11, most of the CYP27A1 variants were able to produce the SLC27A5 product.

### Example 9—Converting (25R)-3α,7α,12α-trihydroxy-5β-cholestan-26-oic acid to (25R)-3α,7α,12α-trihydroxy-5β-cholestanoyl-CoA

Variants of solute carrier family 27 member 5 (SLC27A5) were integrated into wild type yeast strains that had been knocked out for the native yeast CoA-ligase, FAT1. The yeast strains were lysed and CoA ligase activity was detected on (25R)-3α,7α,12α-trihydroxy-5β-cholestan-26-oic acid when expressing different variants of SLC27A5.

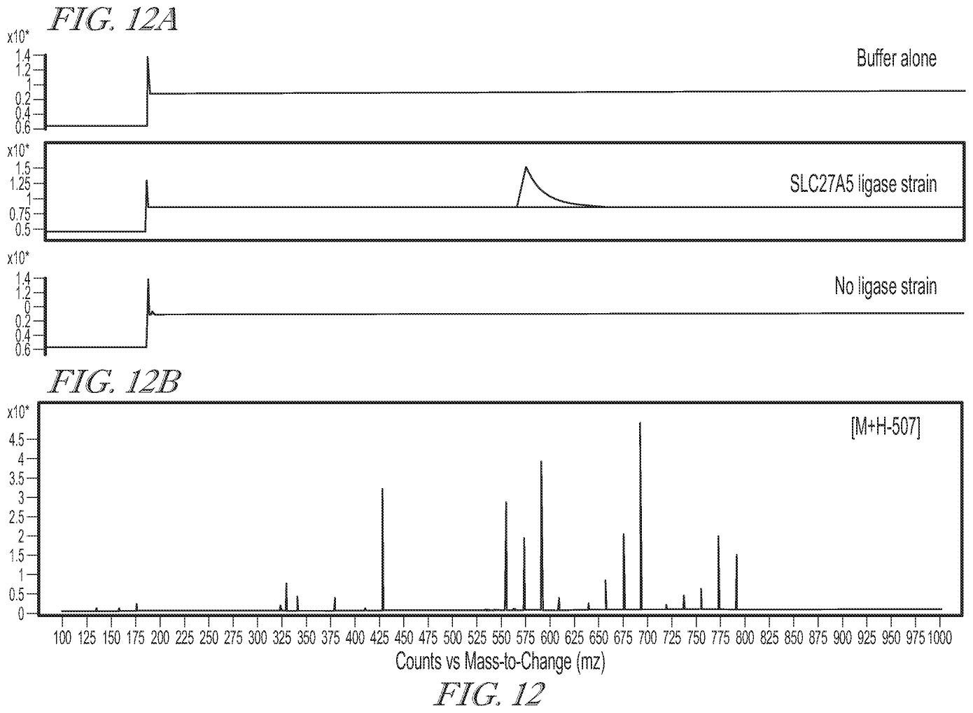

As shown in FIG. 12A, HPLC data shows that there is a peak detected which is specific to ligase expressing strains. Further, as shown in FIG. 12B, mass spec data confirms that there exists a peak that confirms the presence of active ligase in the expressing strains. Additionally, CoA ligase also exhibits activity using 3α,5β,7α,12α,24E-trihydroxy-cholest-24-en-26-oic acid as the substrate.

### Example 10—Converting (25R)-3α,7α-dihydroxy-5β-cholestanoyl-CoA to (25S)-3α,7α-dihydrog-5β-cholestanoyl-CoA

Strains expressing A. thaliana DHCR7, H. sapiens DHCR24, M. musculus CYP7A1, ADX from D. rerio and B. taurus, B. taurus ADR, H. sapiens HSD3B7, M. musculus AKR1D1, M. fuscata AKR1C4, R. norvegicus CYP27A1, H. sapiens SLC27A5, and ACOX2 (from H. sapiens or Ogctolagus cuniculus), were used as background strains to test activity of several alpha-methylacyl-CoA racemases (AMACR). The yeast strains were lysed and (24E)-3α,7α-dihydroxy-5β-cholest-24-enoyl-CoA (product of ACOX2) was measured by LC/MS, since the racemization of (25R)-3α,7α-dihydroxy-5β-cholestanoyl-CoA to (25S)-3α,7α-dihydroxy-5β-cholestanoyl-CoA is difficult to detect.

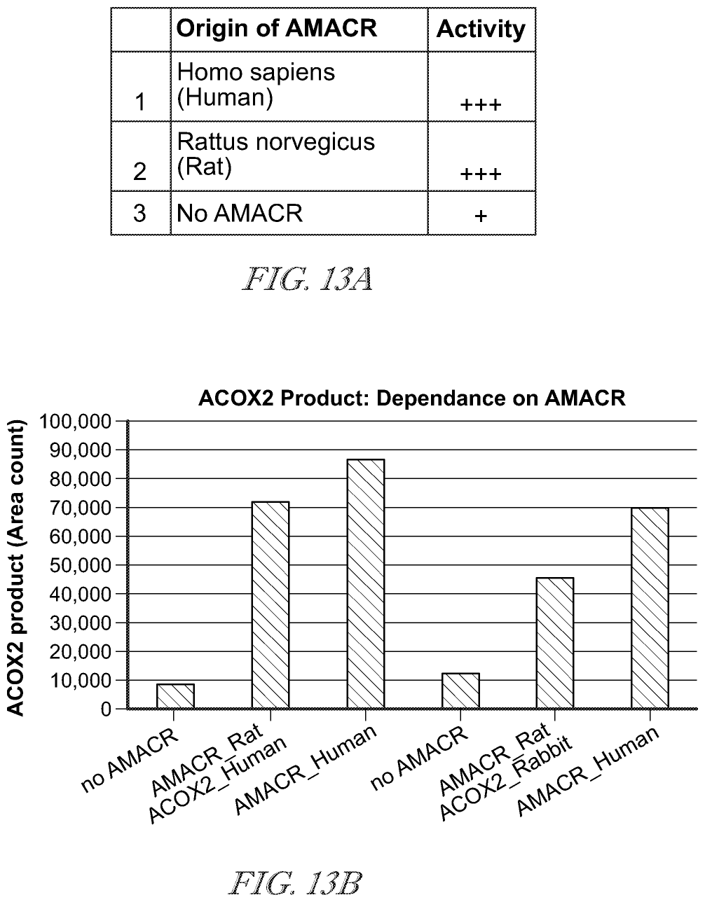

As shown in FIG. 13A, AMACR from both Homo sapiens and Rattus norvegicus produced excellent racemization activity. Further, as shown in FIG. 13B, ACOX2 from Homo sapiens in combination with Homo sapien AMACR produces the most (24E)-3α,7α-dihydroxy-5β-cholest-24-enoyl-CoA.

### Example 11—Converting (25S)-3α,7α-dihydroxy-5β-cholestanoyl-CoA to (24E)-3α,7α-dihydroxy-5β-cholest-24-enoyl-CoA

Strains expressing A. thaliana DHCR7, H. sapiens DHCR24, M. musculus CYP7A1, ADX from D. rerio and B. taurus, B. taurus ADR, H. sapiens HSD3B7, M. musculus AKR1D1, M. fuscata AKR1C4, R. norvegicus CYP27A1, and H. sapiens SLC27A5, and AMACR (from Homo sapiens and Rattus norvegicus), were used as background strains to test activity of different acyl-CoA oxidase 2 (ACOX2). The yeast strains were lysed and (24E)-3α,7α-dihydroxy-5β-cholest-24-enoyl-CoA measured by LC/MS.

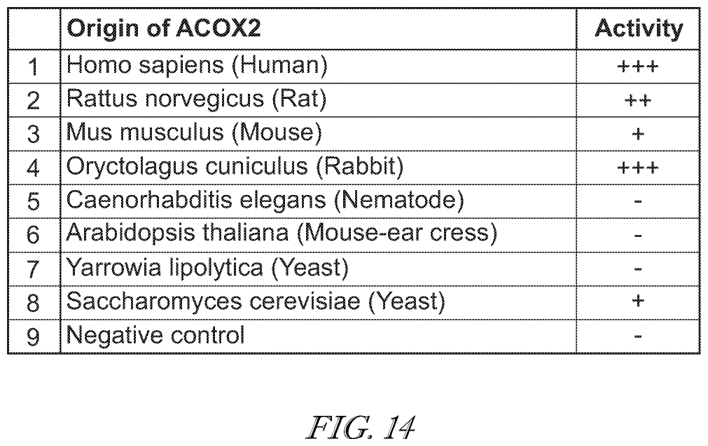

As shown in FIG. 14, ACOX2 from both Homo sapiens and Ogctolagus cuniculus produced the best activity. ACOX2 from Rattus norvegicus, Mus musculus, and Saccharomyces cerevisiae exhibited activity.

### Example 12—Converting (24E)-3α,7α,12α-trihydroxy-5β-cholest-24-enoyl-CoA to 3α,7α,12α-trihydroxy-24-oxo-5β-cholestanoyl-CoA

Strains expressing SLC27A5-CoA ligases were used as background strains to test activity of different hydroxysteroid 17-beta dehydrogenase 4 (HSD17B4). The yeast strains were lysed and in vitro assays conducted with added substrate 3α,5β,7α,12α,24E-trihydroxy-cholest-24-en-26-oic acid (SLC27A5 CoA-ligase activity has been verified on this substrate).

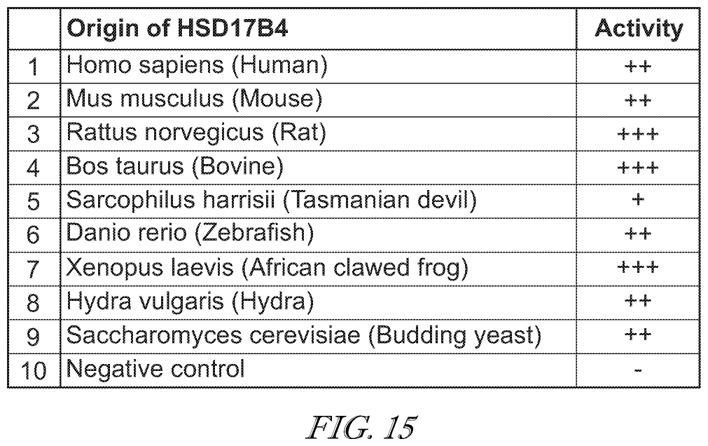

The intermediate product of this bifunctional enzyme HSD17B4, an alcohol, was detected. As shown in FIG. 15, HSD17B4 from Rattus norvegicus, Bos taurus, and Xenopus laevis produced the best activity. HSD17B4 from remaining 6 sources also exhibited activity.

### Example 13—Converting 3α,7α-dihydroxy-24-oxo-5β-cholestangl-CoA to 3α,7α-dihydrog-5β-cholan-24-yl-CoA

Strains expressing A. thaliana DHCR7, H. sapiens DHCR24, M. musculus CYP7A1, ADX from D. rerio and B. taurus, B. taurus ADR, H. sapiens HSD3B7, M. musculus AKR1D1, M. fuscata AKR1C4, R. norvegicus CYP27A1, and H. sapiens SLC27A5, R. norvegicus AMACR, H. sapiens ACOX2, and R. norvegicus HSD17B4 were used as background strains to test activity of sterol carrier protein 2 (SCP2). The background strain was also knocked out for its native yeast gene POT1 which encodes for a 3-ketoacyl-CoA thiolase and expressed Bacteroides fragilis 7α-HSD and Clostridium sardiniense 7β-HSD. Yeast pellets were extracted and subsequently analyzed for relative amounts of UDCA/UDC-CoA product by LC/MS.

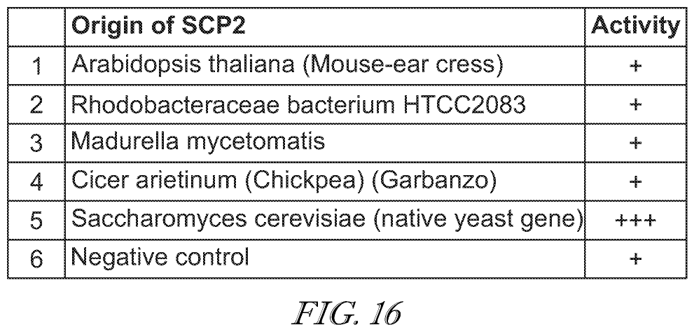

As shown in FIG. 16, SCP2 activity was detected by LCMS in all samples, including negative control, however enhanced activity was observed in the strain overexpressing the native yeast gene POT1.

### Example 14—Converting 3α,7α-dihydrog-5β-cholan-24-oyl-CoA to 3α-hydroxy-7-oxo-5β-cholan-24-oyl-CoA to 3α,7β-dihydroxy-5β-cholan-24-oyl-CoA

Strains expressing S. cerevisiae truncated HMG, A. thaliana DHCR7, H. sapiens DHCR24, M. musculus CYP7A1, ADX from D. rerio and B. taurus, B. taurus ADR, H. sapiens HSD3B7, M. musculus AKR1D1, M. fuscata AKR1C4, R. norvegicus CYP27A1, and H. sapiens SLC27A5, R. norvegicus AMACR, H. sapiens ACOX2, and R. norvegicus HSD17B4, S. cerevisiae SCP2, pot1Δ, pox1Δ, and fox2Δ were used as background strains to determine the working 7alpha and 7beta-hydroxysteroid dehydrogenases, 7α-HSD and 7β-HSD, respectively.

Four variants of 7α-HSD (Escherichia coli (strain K12), Luminiphilus syltensis NORS-1B, Bacteroides fragilis, and Comamonas testosteroni (Pseudomonas testosterone)) were tested in the background strain (in this case also expressing an active C. sardiniense 7(-HSD) for their ability to produce UDC-CoA (also known as 3α,7β-dihydroxy-5β-cholanoyl-CoA having a chemical formula of C₄₅H₇₄N₇O₁₉P₃S with a mass of 1141.40 and a molecular weight of 1142.10).

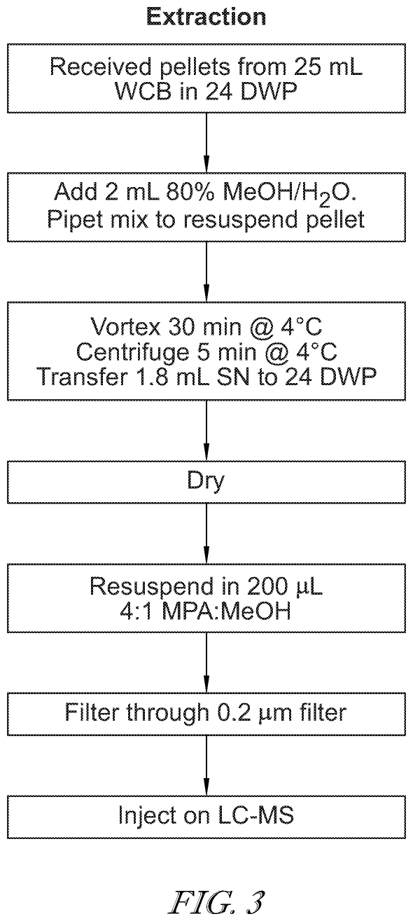

Cell pellets were collected from 25 mL whole cell broth in 24 deep well plates. The cell pellets were re-suspended in a 2 mL 80% Methanol/Water mixture solution, vortexed for 30 minutes at 4° C., centrifuged for 5 minutes at 4° C. at 4000 rpm, and transferred 1.8 mL Supernatant to 24 deep well plate. The resulting pellets were dried and re-suspended in 200 μL of a 4:1 MPA (10 mM ammonium formate in water, pH 6):Methanol solution. This resuspension was filtered through a 0.2 μm filter. This final filtered product was measured by liquid chromatography followed by mass spectrometry for the presence of UDC-CoA. A flow chart showing these steps is shown in FIG. 3.

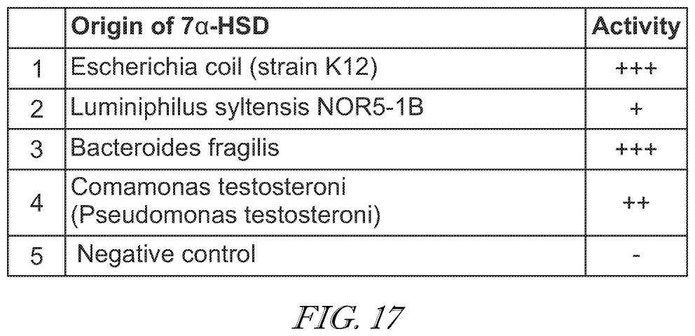

As shown in FIG. 17, 7a-HSD from E. coi and B. fragilis, exhibited significant activity. 7α-HSD from L. syltensis and C. testosterioni showed activity as well.

Four variants of 7β-HSD (Pseudomonas syringae pv. atrofaciens, Pseudomonas cruicapapayae, Drosophila persimilis (Fruit fly), and Clostridium sardiniense)) were also tested in a background strain (in this case also expressing an active B. fragilis 7α-HSD) for their ability to produce UDC-CoA. The same procedure described above was used.

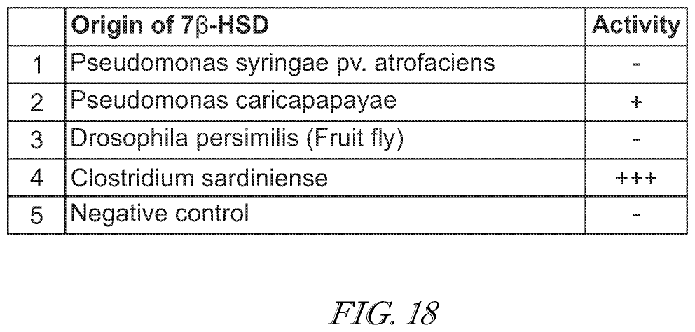

As shown in FIG. 18, 7β-HSD from Clostridium sardiniense exhibited the best activity. 7β-HSD from Pseudomonas caricapapayae also exhibited some activity.

### Example 15—Confirmation that UDC-CoA was Made

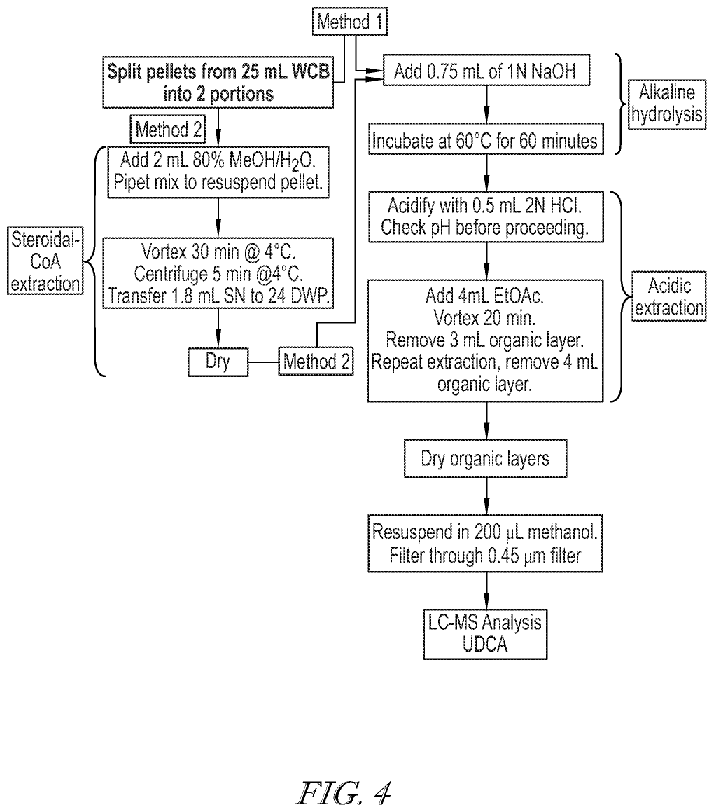

In order to verify that UDC-CoA from Example 14 was indeed produced, two additional methods of processing samples for use in mass spectrometry were conducted. As seen in FIG. 4, the initial pellets were split into two samples. The first sample was washed with 2 mL of 80% Methanol/H₂O, vortexed, centrifuged, transferred and dried.

The first sample, as with the second sample, went through the same processing from this point on.

750 μL of 1N NaOH were added to the pellets and incubated for 60 minutes at 60° C. The sample was then acidified with 500 μL of 2N HCl. 4 mL of EtOAc was added and vortexed for 20 minutes. 3 mL of the organic layer was removed and dried. This was resuspended in 200 μL methanol and filtered through a 0.45 μM filter.

Both direct hydrolysis of the pellets and the indirect hydrolysis of the steroidal-CoA extracts resulted in the detectable UDCA, CDCA, (24E)-3α,7α-dihydroxy-cholest-24-enoic acid, and 3α,7α(-dihydroxy-5β-cholestanoic acid. Direct hydrolysis of the pellets seems to yield more.

### Example 16—Combination of Thiolase/7α-HSD/7β-HSD

Strains expressing S. cerevisiae truncated HMG, A. thaliana DHCR7, H. sapiens DHCR24, M. musculus CYP7A1, H. sapiens HSD3B7, M. musculus AKR1D1, M. fuscata AKR1C4, R. norvegicus CYP27A1, and H. sapiens SLC27A5, R. norvegicus AMACR, H. sapiens ACOX2, and R. norvegicus HSD17B4, pot1A, pox1A, and fox2A, were used as background strains to determine the best combination of thiolase/SCP2, 7α-HSD, and 7β-HSD.

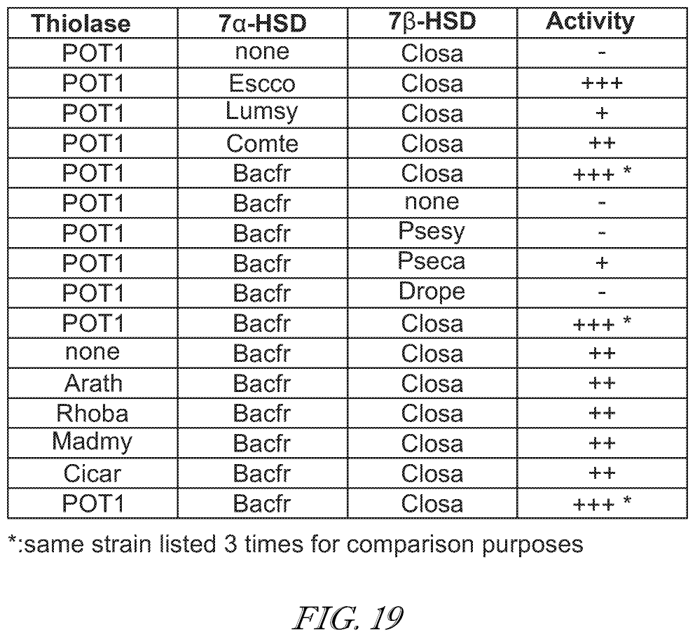

The strains were then tested by GC/MS for its ability to produce UDCA/UDC-CoA. As seen in FIG. 19, the combination of S. cerevisiae POT1 Thiolase, E. coli 7α-HSD, and C. sardiniense 7β-HSD and S. cerevisiae POT1 Thiolase, B. fragilis 7α-HSD, and C. sardiniense 7β-HSD lead to the greatest amounts of UDCA/UDC-CoA production. Other combinations produced detectable levels of UDCA/UDC-CoA production, as seen in FIG. 19.

### Example 17—Identification of Engvnes that Convert Sugar to Cholic Acid and Generating Strains that can Make Cholic Acid

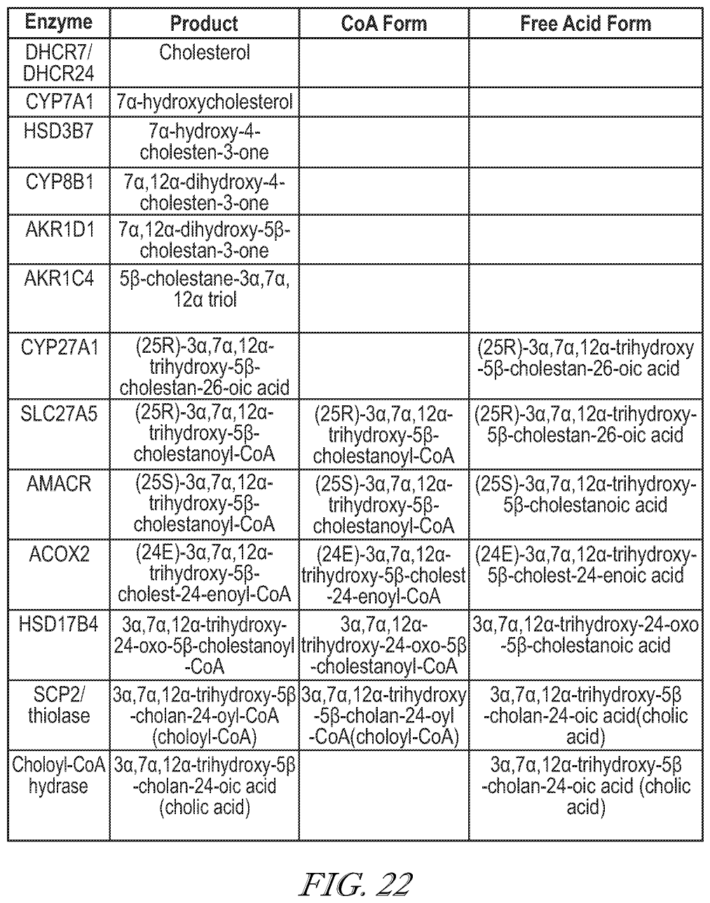

Eleven heterologous enzymes (from the perspective of a Saccharomyces cerevisiae) were identified as possible enzymes that could be used to make cholic acid from cholesterol. See e.g., FIG. 22. Two (2) additional enzymes were also identified as possible enzymes that could be used to convert sugar to cholesterol. See e.g., FIG. 2.

Genes encoding these enzymes were synthesized and then cloned into yeast expression vectors suitable for integration into the yeast genome. These integration constructs were subsequently transformed into Saccharomyces cerevisiae using standard yeast chemical transformation protocol, utilizing Lithium Acetate and PEG (3350). The transformed yeast were grown to mid log phase, then centrifuged at 4000 rpm with the supernatant removed. Pellets were washed with water and centrifuged again. The resulting pellet was resuspended in master mix containing 100 mM lithium acetate, 40% PEG (MW 3,350), 0.35 mg/ml carrier DNA (sheared salmon sperm DNA), and 50 to 500 ng of DNA to be transformed. The cell suspension was then incubated at 30° C. for 30 minutes, followed by at 45 minute heat shock at 42° C. At this point, nutritional selection was plated, while antifungal selection underwent a 4 hr to overnight recovery in rich yeast media before plating on agar containing the antifungal drug. Plates were then incubated at 30° C. for 2 to 3 days. After colonies were formed, proper integrations were verified by colony PCR before using strain in experiments.

Table 2 shows representative genes that were expressed in the yeast strains and the genetic origin of the enzymes that exhibited the best activity. Genes from other sources were also found to be active, but are not represented on Table 2.

TABLE 2

| Gene/enzyme | SEQ ID NO(s). | Source of Variants |
|---|---|---|
| ADR | 239 | Bovine |
| ADX | 241, 243, 245, 247, | Bovine, Zebrafish, |
| | 249, 251, 253, 255, | human |
| | 257, 259, 261 | |
| DHCR7 | 1 | Arabidopsis |
| DHCR24 | 21, 23, 25, 27, 45, 47 | Human, Bovine, |
| | | Zebrafish |
| CYP7A1 | 53, 65, 67, 69, 71, 73, | Mouse |
| | 75, 77, 79 | |
| HSD3B7 | 81, | Human |
| AKR1D1 | 91 | Mouse |
| AKR1C4 | 101 | Macaca fuscata |
| CYP27A1 | 125, 129, 131 | Rat, Mouse, Bovine |
| SLC27A5 | 139 | Human |
| AMACR | 145, 147 | Rat, Human |
| ACOX2 | 159, 165 | Human, Rabbit |
| HSD17B4 | 179, 183, 189 | Rat, Bovine, Xenopus |
| SCP2 | 203 | Yeast (POT1) |
| CYP8B1 | 269 | Mouse |

Strains with the ability to produce cholesterol were genetically engineered to further express CYP7A1, ADX (2 variants), ADR, and HSD3B7. The activities of CYP7A1 and HSD3B7 were demonstrated as described in Examples 3 and 4.

### Example 18—Converting 7α-hydroxy-4-cholesten-3-one to 7α,12α-dihydrog-4-cholesten-3-one

Strains expressing A. thaliana DHCR7, H. sapiens DHCR24 were genetically engineered to further express M. musculus CYP7A1, ADX (from D. rerio and B. taurus), B. taurus ADR, H. sapiens HSD3B7, and CYP8B1.

The strains were tested for their abilities to produce 7α,12α-dihydroxy-4-cholesten-3-one from 7α-hydroxy-4-cholesten-3-one.

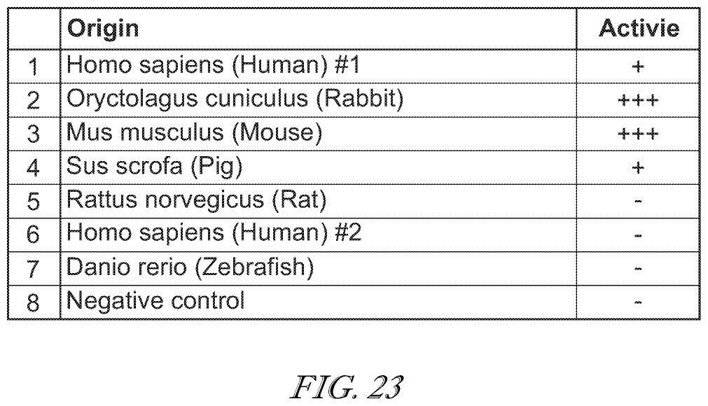

As shown in FIG. 23, CYP8B1 from Mus musculus and Ogctolagus cuniculus exhibited the best activity. CYP8B1 from Homo sapiens and Sus scrofa also exhibited activity.

### Example 19—Confirmation that Choloyl-CoA was Made

Strains expressing S. cerevisiae truncated HMG, A. thaliana DHCR7, H. sapiens DHCR24, M. musculus CYP7A1, B. taurus ADX, B. taurus ADR, H. sapiens HSD3B7, M. musculus AKR1D1, M. fuscata AKR1C4, R. norvegicus CYP27A1, and H. sapiens SLC27A5, R. norvegicus AMACR, H. sapiens ACOX2, R. norvegicus HSD17B4, and S. cerevisiae SCP2 were used as background strains to determine the working CYP8B1.

One variant of CYP8B1 was tested (Mus musculus) in the background strain for its ability to produce choloyl-CoA (also known as 3α,7α,12α-trihydroxy-5(-cholan-24-oyl-CoA, having a chemical formula of C₄₅H₇₄N₇O₂₀P₃S with a mass of 1157.4 and a molecular weight of 1158.1). The hydrolyzed acid form of choloyl-CoA, cholic acid (also known as 3α,7α,12α-trihydroxy-5β-cholan-24-oic acid, having a chemical formula of C₂₄H₄₀O₅ with a mass of 408.3 and a molecular weight of 408.58) was the measureable product.

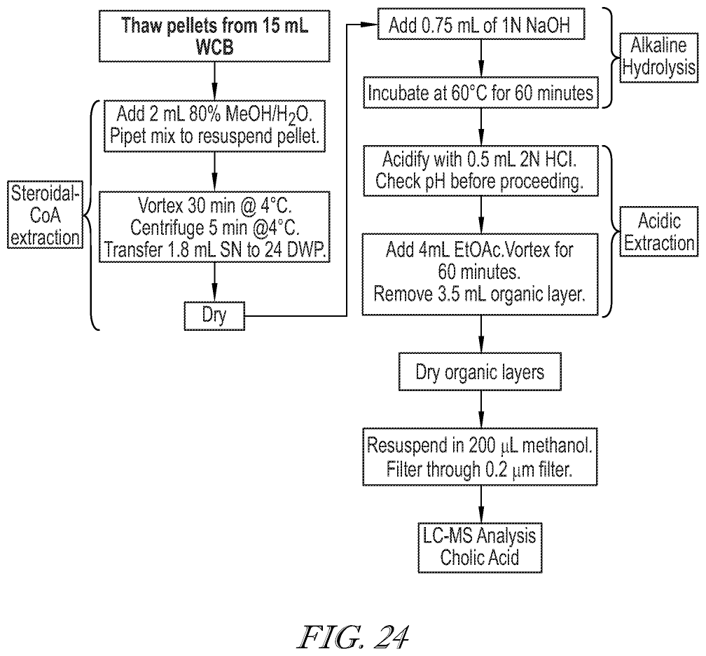

Cell pellets were collected from 15 mL whole cell broth in 24 deep well plates. The cell pellets were re-suspended in a 2 mL 80% Methanol/Water mixture solution, vortexed for 30 minutes at 4° C., centrifuged for 5 minutes at 4° C. at 4000 rpm, and 1.8 mL supernatant was transferred to 24 deep well plate. The supernatant was dried overnight at 40° C. on centrivap. The dried extracts were hydrolyzed with 750 μL 1N NaOH at 60° C. for 1 hour with vortexing, followed by acidification with 500 μL 2N HCl. The acidified samples were extracted with 4 mL ethyl acetate. 3.5 mL of the organic layer was transferred to a 24 deep well plate and dried at 45° C. on centrivap. The dried extracts were resuspended in 200 μL methanol and filtered through a 0.2 μm filter. This final filtered product was measured by liquid chromatography followed by mass spectrometry for the presence of cholic acid (hydrolyzed choloyl-CoA). A flow chart showing these steps is shown in FIG. 24.

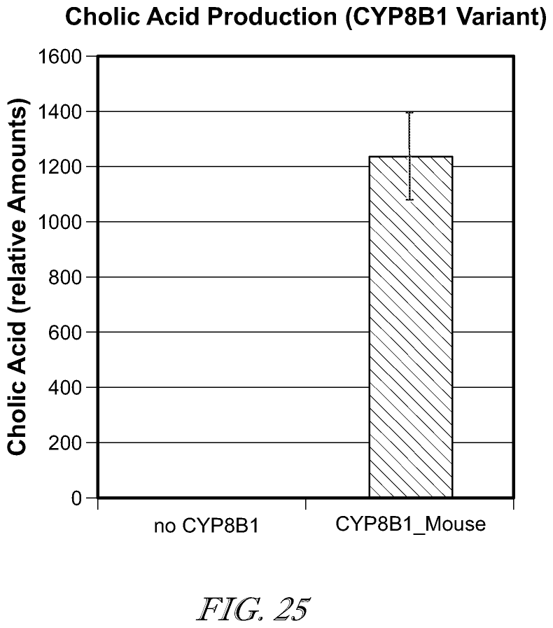

As shown in FIG. 25, the CYP8B1 from Mus musculus was active and produced choloyl-CoA (cholic acid detected). No cholic acid was detected in the strain lacking the CYP8B1 enzyme.

1. A genetically-modified cell capable of producing UDCA or a UDCA precursor comprising at least one heterologous polynucleotide encoding an enzyme involved in a metabolic pathway that converts sugar to UDCA or a UDCA precursor.

2. The cell of claim 1, comprising at least two heterologous polynucleotides, each encoding an enzyme involved in a metabolic pathway that converts sugar to UDCA or a UDCA precursor, wherein the encoded enzymes are operably connected along the metabolic pathway.

3. The cell of claim 1 or 2, wherein the UDCA precursor is desmosterol; cholesterol; 7-alpha-hydroxycholesterol; 7α-hydroxy-4-cholesten-3-one; 7α-hydroxy-5β-cholestan-3-one; 5β-cholestane-3α,7α-diol; (25R)-3α,7α-dihydroxy-5β-cholestanoic acid; (25R)-3α,7α-dihydroxy-5β-chole stanoyl-CoA; (25S)-3α,7α-dihydroxy-5β-cholestanoyl-CoA; (24E) -3α,7α-dihydroxy-5β-cholest-24-enoyl-CoA; 3α,7α-dihydroxy-24-oxo-5β-cholestanoyl-CoA; 3α,7α-dihydroxy-5β-cholan-24-oyl-CoA; 3α-hydroxy-7-oxo-5β-cholan-24-oyl-CoA; 3α,7β-dihydroxy-5β-cholan-24-oyl-CoA; 7α,12α-dihydroxy-4-cholesten-3-one; 7α,12α-dihydroxy-5β-cholestan-3-one; 5β-cholestane-3α,7α,12α-triol; (25R)-3α,7α,12α-trihydroxy-5β-cholestan-26-oic acid; (25R)-3α,7α,12α-trihydroxy-5β-cholestanoyl-CoA; (25S)-3α,7α,12α-trihydroxy-5β-cholestanoyl-CoA; (24E)-3α,7α,12α-trihydroxy-5β-cholest-24-enoyl-CoA; 3α,7α,12α-trihydroxy-24-oxo-5β-cholestanoyl-CoA; 3α,7α,12α-trihydroxy-5β-cholan-24-oyl-CoA; or cholic acid.

4. The cell of any one of claims 1-3, wherein the encoded enzyme is DHCR7, DHCR24, CYP7A1, HSD3B7, CYP8B1, AKR1D1, AKR1C9, AKR1C4, CYP27A1, SLC27A5, FAT1, AMACR, ACOX2, PDX1, HSD17B4, FOX2, SCP2, POT1, ERG10, 7α-HSD, 7β-HSD, or choloyl-CoA hydrolase.

5. The cell of any one of claims 1-4, wherein the encoded enzyme is involved in the metabolic pathway that converts sugar to cholesterol.

6. The cell of any one of claims 1-4, wherein the encoded enzyme is involved in the metabolic pathway that converts cholesterol to CDC-CoA.

7. The cell of any one of claims 1-4, wherein the encoded enzyme is involved in the metabolic pathway that converts cholesterol to cholic acid.

8. The cell of any one of claims 1-4, wherein the encoded enzyme is involved in the metabolic pathway that converts CDC-CoA to UDCA.

9. The cell of any one of claims 1-5, wherein the encoded enzyme is:
DHCR7 and is encoded by a polynucleotide comprising a nucleic acid sequence that is substantially identical to any one of SEQ ID NOs: 2, 4, 6, 8, 10, or 12; or
DHCR24 and is encoded by a polynucleotide comprising a nucleic acid sequence that is substantially identical to any one of SEQ ID NOs: 14, 15, 16, 18, 19, 20, 22, 23, 24, 26, 27, 28, 30, 31, 32, 34, 35, 36, 38, 39, 40, 42, 44, 46, or 48.

10. The cell of any one of claim 1-4 or 6-7, wherein the encoded enzyme is:
CYP7A1 and is encoded by a polynucleotide comprising a nucleic acid sequence that is substantially identical to any one of SEQ ID NOs: 50, 52, 54, 56, 58, 60, 62, 64, 66, 68, 70, 72, 74, 76, 78, or 80;
HSD3B7 and is encoded by a polynucleotide comprising a nucleic acid sequence that is substantially identical to any one of SEQ ID NOs: 82, 84, 86, or 88;
CYP8B1 and is encoded by a polynucleotide comprising a nucleic acid sequence that is substantially identical to any one of SEQ ID NOs: 266, 268, 270, 272, 274, 276, or 278;
AKR1D1 and is encoded by a polynucleotide comprising a nucleic acid sequence that is substantially identical to any one of SEQ ID NOs: 90, 92, 94, or 96;
AKR1C9 and is encoded by a polynucleotide comprising a nucleic acid sequence that is substantially similar to SEQ ID NO: 98;
AKR1C4 and is encoded by a polynucleotide comprising a nucleic acid sequence that is substantially identical to any one of SEQ ID NOs: 100, 102, 104, 106, 108, 110, 112, 114, 116, 118, 120, or 122;
CYP27A1 and is encoded by a polynucleotide comprising a nucleic acid sequence that is substantially identical to any one of SEQ ID NOs: 124, 126, 128, 130, 132, 134, 136, or 138;
SLC27A5 and is encoded by a polynucleotide comprising a nucleic acid sequence that is substantially identical to SEQ ID NOs: 140 or 142;
FAT1 and is encoded by a polynucleotide comprising a nucleic acid sequence that is substantially identical to SEQ ID NO: 144;
AMACR and is encoded by a polynucleotide comprising a nucleic acid sequence that is substantially identical to any one of SEQ ID NOs: 146, 148, 150, 152, 154, 156, or 158;
ACOX2 and is encoded by a polynucleotide comprising a nucleic acid sequence that is substantially identical to any one of SEQ ID NOs: 160, 162, 164, 166, 168, 170, 172, or 174;
PDX1 and is encoded by a polynucleotide comprising a nucleic acid sequence that is substantially identical to SEQ ID NO: 176;
HSD17B4 and is encoded by a polynucleotide comprising a nucleic acid sequence that is substantially identical to any one of SEQ ID NOs: 178, 180, 182, 184, 186, 188, 190, or 192;
FOX2 and is encoded by a polynucleotide comprising a nucleic acid sequence that is substantially identical to SEQ ID NO: 194;
SCP2 and is encoded by a polynucleotide comprising a nucleic acid sequence that is substantially identical to any one of SEQ ID NOs: 196, 198, 200, or 202;
POT1 and is encoded by a polynucleotide comprising a nucleic acid sequence that is substantially identical to SEQ ID NO: 204; or
ERG10 and is encoded by a polynucleotide comprising a nucleic acid sequence that is substantially identical to SEQ ID NO: 206.

11. The cell of claim 8, wherein the encoded enzyme is:
7α-HSD and is encoded by a polynucleotide comprising a nucleic acid sequence that is substantially identical to any one of SEQ ID NOs: 208, 210, 212, or 214;
7β-HSD is encoded by a polynucleotide comprising a nucleic acid sequence that is substantially identical to any one of SEQ ID NOs: 216, 218, 220, or 222; and
choloyl-CoA hydrolase is encoded by a polynucleotide comprising a nucleic acid sequence that is substantially identical to any one of SEQ ID NOs: 224, 226, 228, or 230.

12. The cell of any one of claims 1-11, further comprising a heterologous polynucleotide encoding ADR, ADX, and/or a truncated HMG

13. The cell of any one of claims 1-12, wherein the cell is a microorganism or part of a microorganism.

14. The cell of any one of claims 1-13, wherein the cell is bacterium or a yeast.

15. The cell of any one of claims 1-14, wherein the cell is Saccharomyces cerevisiae.

16. A method of making UDCA or a UDCA precursor, the method comprising:
(a) contacting a substrate with the genetically-modified cell of any one of claims 1-15; and
(b) growing the cell to make UDCA or UDCA precursor.

17. The method of claim 16, further comprising isolating the UDCA or UDCA precursor from the cell.

18. The use of UDCA or UDCA precursor made using the method of claim 16 or 17 for the manufacture of a medicament for the treatment of a disease or a symptom of a disease.

19. The use of claim 19, wherein the disease or symptom of a disease is gallstones, primary biliary cirrhosis, cystic fibrosis, impaired bile flow, intrahepatic cholestasis of pregnancy, and/or cholelithiasis.

20. A medicament comprising UDCA or UDCA precursor made using the method of claim 16 or 17.

21. A method of treating a disease or symptom of a disease comprising administering UDCA or a UDCA precursor made using the method of claim 15 or 16 to a subject in need thereof.

22. The method of claim 21 wherein the disease or symptom of a disease is gallstones, primary biliary cirrhosis, cystic fibrosis, impaired bile flow, intrahepatic cholestasis of pregnancy, and/or cholelithiasis.

23. An isolated polynucleotide encoding at least one enzyme involved in a metabolic pathway that converts sugar to UDCA or a UDCA precursor.

24. The polynucleotide of claim 23, wherein the encoded enzyme is DHCR7, DHCR24, CYP7A1, HSD3B7, CYP8B1, AKR1D1, AKR1C9, AKR1C4, CYP27A1, SLC27A5, FAT1, AMACR, ACOX2, PDX1, HSD17B4, FOX2, SCP2, POT1, ERG10, 7α-HSD, 7β-HSD, or choloyl-CoA hydrolase.

25. The polynucleotide of claim 23 or 24, wherein the encoded enzyme is involved in the metabolic pathway that converts sugar to cholesterol.

26. The polynucleotide of claim 23 or 24, wherein the encoded enzyme is involved in the metabolic pathway that converts cholesterol to CDC-CoA.

27. The polynucleotide of claim 23 or 24, wherein the encoded enzyme is involved in the metabolic pathway that converts cholesterol to cholic acid.

28. The polynucleotide of claim 23 or 24, wherein the encoded enzyme is involved in the metabolic pathway that converts CDC-CoA to UDCA.

29. The polynucleotide of any one of claims 23-25, wherein the encoded enzyme is:
DHCR7 and the polynucleotide comprises a nucleic acid sequence that is substantially identical to any one of SEQ ID NOs: 2, 4, 6, 8, 10, or 12; or
DHCR24 and the polynucleotide comprises a nucleic acid sequence that is substantially identical to any one of SEQ ID NOs: 14, 15, 16, 18, 19, 20, 22, 23, 24, 26, 27, 28, 30, 31, 32, 34, 35, 36, 38, 39, 40, 42, 44, 46, or 48.

30. The polynucleotide of any one of claims 23-24 and 26-27, wherein the encoded enzyme is:
CYP7A1 and the polynucleotide comprises a nucleic acid sequence that is substantially identical to any one of SEQ ID NOs: 50, 52, 54, 56, 58, 60, 62, 64, 66, 68, 70, 72, 74, 76, 78, or 80;
HSD3B7 and the polynucleotide comprises a nucleic acid sequence that is substantially identical to any one of SEQ ID NOs: 82, 84, 86, or 88;
CYP8B1 and the polynucleotide comprises a nucleic acid sequence that is substantially identical to any one of SEQ ID NOs: 266, 268, 270, 272, 274, 276, or 278;
AKR1D1 and the polynucleotide comprises a nucleic acid sequence that is substantially identical to any one of SEQ ID NOs: 90, 92, 94, or 96;
AKR1C9 and the polynucleotide comprises a nucleic acid sequence that is substantially similar to SEQ ID NO: 98;
AKR1C4 and the polynucleotide comprises a nucleic acid sequence that is substantially identical to any one of SEQ ID NOs: 100, 102, 104, 106, 108, 110, 112, 114, 116, 118, 120, or 122;
CYP27A1 and the polynucleotide comprises a nucleic acid sequence that is substantially identical to any one of SEQ ID NOs: 124, 126, 128, 130, 132, 134, 136, or 138;
SLC27A5 and the polynucleotide comprises a nucleic acid sequence that is substantially identical to SEQ ID NOs: 140 or 142;
FAT1 and the polynucleotide comprises a nucleic acid sequence that is substantially identical to SEQ ID NO: 144;
AMACR and the polynucleotide comprises a nucleic acid sequence that is substantially identical to any one of SEQ ID NOs: 146, 148, 150, 152, 154, 156, or 158;
ACOX2 and the polynucleotide comprises a nucleic acid sequence that is substantially identical to any one of SEQ ID NOs: 160, 162, 164, 166, 168, 170, 172, or 174;
PDX1 and the polynucleotide comprises a nucleic acid sequence that is substantially identical to SEQ ID NO: 176;
HSD17B4 and the polynucleotide comprises a nucleic acid sequence that is substantially identical to any one of SEQ ID NOs: 178, 180, 182, 184, 186, 188, 190, or 192;
FOX2 and the polynucleotide comprises a nucleic acid sequence that is substantially identical to SEQ ID NO: 194;
SCP2 and the polynucleotide comprises a nucleic acid sequence that is substantially identical to any one of SEQ ID NOs: 196, 198, 200, or 202;
POT1 and the polynucleotide comprises a nucleic acid sequence that is substantially identical to SEQ ID NO: 204; or
ERG10 and the polynucleotide comprises a nucleic acid sequence that is substantially identical to SEQ ID NO: 206.

31. The polynucleotide of any one of claims 23-24 and 28, wherein the encoded enzyme is:
7α-HSD and the polynucleotide comprises a nucleic acid sequence that is substantially identical to any one of SEQ ID NOs: 208, 210, 212, or 214;
7β-HSD and the polynucleotide comprises a nucleic acid sequence that is substantially identical to any one of SEQ ID NOs: 216, 218, 220, or 222; and
choloyl-CoA hydrolase and the polynucleotide comprises a nucleic acid sequence that is substantially identical to any one of SEQ ID NOs: 224, 226, 228, or 230.

32. A vector comprising a nucleic acid encoding at least one enzyme involved in a metabolic pathway that converts sugar to UDCA or a UDCA precursor.

33. The vector of claim 32, wherein the encoded enzyme is DHCR7, DHCR24, CYP7A1, HSD3B7, CYP8B1, AKR1D1, AKR1C9, AKR1C4, CYP27A1, SLC27A5, FAT1, AMACR, ACOX2, PDX1, HSD17B4, FOX2, SCP2, POT1, ERG10, 7α-HSD, 7β-HSD, or choloyl-CoA hydrolase.

34. The vector of claim 32 or 33, wherein the encoded enzyme is involved in the metabolic pathway that converts sugar to cholesterol.

35. The vector of claim 32 or 33, wherein the encoded enzyme is involved in the metabolic pathway that converts cholesterol to CDC-CoA.

36. The vector of claim 32 or 33, wherein the encoded enzyme is involved in the metabolic pathway that converts cholesterol to cholic acid.

37. The vector of claim 32 or 33, wherein the encoded enzyme is involved in the metabolic pathway that converts CDC-CoA to UDCA.

38. The vector of any one of claims 32-34, wherein the encoded enzyme is:
DHCR7 and the vector comprises a nucleic acid sequence that is substantially identical to any one of SEQ ID NOs: 2, 4, 6, 8, 10, or 12; or
DHCR24 and the vector comprises a nucleic acid sequence that is substantially identical to any one of SEQ ID NOs: 14, 15, 16, 18, 19, 20, 22, 23, 24, 26, 27, 28, 30, 31, 32, 34, 35, 36, 38, 39, 40, 42, 44, 46, or 48.

39. The vector of any one of claims 32-33 and 35-36, wherein the encoded enzyme is:
CYP7A1 and the vector comprises a nucleic acid sequence that is substantially identical to any one of SEQ ID NOs: 50, 52, 54, 56, 58, 60, 62, 64, 66, 68, 70, 72, 74, 76, 78, or 80;
HSD3B7 and the vector comprises a nucleic acid sequence that is substantially identical to any one of SEQ ID NOs: 82, 84, 86, or 88;
CYP8B1 and the vector comprises a nucleic acid sequence that is substantially identical to any one of SEQ ID NOs: 266, 268, 270, 272, 274, 276, or 278;
AKR1D1 and the vector comprises a nucleic acid sequence that is substantially identical to any one of SEQ ID NOs: 90, 92, 94, or 96;
AKR1C9 and the vector comprises a nucleic acid sequence that is substantially identical to SEQ ID NO: 98;
AKR1C4 and the vector comprises a nucleic acid sequence that is substantially identical to any one of SEQ ID NOs: 100, 102, 104, 106, 108, 110, 112, 114, 116, 118, 120, or 122;
CYP27A1 and the vector comprises a nucleic acid sequence that is substantially identical to any one of SEQ ID NOs: 124, 126, 128, 130, 132, 134, 136, or 138;
SLC27A5 and the vector comprises a nucleic acid sequence that is substantially identical to SEQ ID NOs: 140 or 142;
FAT1 and the vector comprises a nucleic acid sequence that is substantially identical to SEQ ID NO: 144;
AMACR and the vector comprises a nucleic acid sequence that is substantially identical to any one of SEQ ID NOs: 146, 148, 150, 152, 154, 156, or 158;
ACOX2 and the vector comprises a nucleic acid sequence that is substantially identical to any one of SEQ ID NOs: 160, 162, 164, 166, 168, 170, 172, or 174;
PDX1 and the vector comprises a nucleic acid sequence that is substantially identical to SEQ ID NO: 176;
HSD17B4 and the vector comprises a nucleic acid sequence that is substantially identical to any one of SEQ ID NOs: 178, 180, 182, 184, 186, 188, 190, or 192;
FOX2 and the vector comprises a nucleic acid sequence that is substantially identical to SEQ ID NO: 194;
SCP2 and the vector comprises a nucleic acid sequence that is substantially identical to any one of SEQ ID NOs: 196, 198, 200, or 202;
POT1 and the vector comprises a nucleic acid sequence that is substantially identical to SEQ ID NO: 204; or
ERG10 and the vector comprises a nucleic acid sequence that s substantially identical to SEQ ID NO: 206.

40. The vector of any one of claims 32-33 and 37, wherein the encoded enzyme is:
7α-HSD and the vector comprises a nucleic acid sequence that is substantially identical to any one of SEQ ID NOs: 208, 210, 212, or 214;
7β-HSD and the vector comprises a nucleic acid sequence that is substantially identical to any one of SEQ ID NOs: 216, 218, 220, or 222; and
choloyl-CoA hydrolase and the vector comprises a nucleic acid sequence that is substantially identical to any one of SEQ ID NOs: 224, 226, 228, or 230.

41. A method of making a genetically-modified cell capable of synthesizing UDCA or a UDCA precursor, the method comprising:
(a) contacting a cell with at least one heterologous polynucleotide encoding an enzyme involved in a metabolic pathway that converts sugar to UDCA or a UDCA precursor; and
(b) growing the cell so that said polynucleotide is inserted into said microorganism.

42. The method of claim 41, wherein said cell is a bacterium or a yeast cell.

43. The method of claim 41 or 42, wherein the cell is a Saccharomyces cerevisiae cell.

44. A composition comprising UDCA or a UDCA precursor, a free acid or CoA thereof, or a pharmaceutically-acceptable derivative or prodrug thereof, the UDCA, UDCA precursor, free acid or CoA thereof, or pharmaceutically-acceptable derivative or prodrug thereof produced by a method of claim 16 or 17.
